(19) United States
(12) Patent Application Publication
(10) Pub. No.: US 2017/0130248 A1
(43) Pub. Date: May 11, 2017

(54) Byosynthetic Production of Acyl Amino Acids

(71) Applicant: Evonik Degussa GmbH, Essen (DE)

(72) Inventors: LIV REINECKE, Essen (DE); STEFFEN SCHAFFER, Herten (DE); KATRIN GRAMMANN, Oer-Erkenschwick (DE); MAIK OLFERT, Recklinghausen (DE); NICOLE DECKER, Recklinghausen (DE); NILS ARTO, Marl (DE); HANS-GEORG HENNEMANN, Bedburg (DE)

(73) Assignee: Evonik Degussa GmbH, Essen (DE)

(21) Appl. No.: 15/312,627

(22) PCT Filed: Apr. 28, 2015
(86) PCT No.: PCT/EP2015/059128

§ 371 Date: Nov. 19, 2016

(30) Foreign Application Priority Data
May 23, 2014 (EP) 14169650.0
Aug. 25, 2014 (EP) 14182110.8

(51) Int. Cl. C12P 13/00; C12N 9/88; C12N 9/00; C12N 9/10

(52) U.S. Cl. CPC C12P 13/005; C12N 9/1014; C12N 9/1029; C12N 9/88; C12N 9/93; C12Y 203/01065; C12Y 602/01001; C12Y 201/02001; C12Y 401/02005

(57) ABSTRACT

The present invention relates to a cell for producing acyl glycinates wherein the cell is genetically modified to comprise
at least a first genetic mutation that increases the expression relative to the wild type cell of an amino acid-N-acyl-transferase,
at least a second genetic mutation that increases the expression relative to the wild type cell of an acyl-CoA synthetase, and
at least a third genetic mutation that decreases the expression relative to the wild type cell of at least one enzyme selected from the group consisting of an enzyme of the glycine cleavage system, glycine hydroxymethyltransferase (GlyA) and threonine aldolase (LtaE).

### FIELD OF THE INVENTION

The present invention relates to biotechnological methods and cells for producing at least one acyl amino acid. In particular, the acyl amino acid is an acyl glycinate from at least one fatty acid.

### BACKGROUND OF THE INVENTION

Acyl amino acids are a class of surface-active agents with a variety of uses, for example as detergents for washing purposes, emulsifiers in food products and as essential ingredients in various personal care products such as shampoos, soaps, moisturizing agents and the like. In addition to having both hydrophobic and hydrophilic regions, a prerequisite for use as a surfactant, the compounds are made of naturally occurring molecules, more specifically amino acids and fatty acids, which are not only non-hazardous and environmentally acceptable but may be readily produced at a large scale using inexpensive biological raw materials. In pharmacological research, acyl amino acids are used as neuromodulators and probes for new drug targets.

Acyl amino acids have been isolated from a multitude of biological sources and are believed to have a range of functions, including for example as signalling molecules in mammalian tissues. Also, identification of endogenous acyl amino acids based on a targeted lipidomics approach, allow them to be used as building blocks for antibiotics in bacterial cultures. For example, since cyclic AMP directly activates NasP, an N-acyl amino acid antibiotic biosynthetic enzyme cloned from an uncultured beta-proteobacterium, these N-acyl amino acids can be easily used in antibiotic production. They are also used as compounds involved in bacterial protein sorting.

Conventionally, acyl amino acids have been produced at an industrial scale starting with materials derived from petrochemicals. More specifically, activated fatty acids provided in the form of acid chlorides may be used to acylate amino acids in an aqueous alkaline medium as described in GB1483500. Shortcomings of such approaches include the need to add hazardous chemicals such as sulphuric acid or anhydrides thereof. Other synthetic approaches are associated with the accumulation of by-products such as chloride salts which have undesirable effect on surfactancy.

An example of a useful acyl amino acid is fatty acyl glycinate which is used as a surfactant in several personal care applications, because of its ability to provide higher order skin benefits. Production of fatty acyl glycinates synthesized by microbial fermentation would allow for a sustainable means of production.

A range of biotechnological routes towards production of acyl amino acids has been described in the art. However, none of them is adequate for commercial large-scale production of acyl amino acids owing to low yields, insufficient purities and the need for multi-step purification procedures. In particular, only a small proportion of the carbon substrates fed to biotechnologically useful organisms is actually converted to the desired product, whilst much of it is consumed by reactions of the primary metabolism.

Another problem associated with the available biotechnological routes is the fact that a mixture of products is obtained and thus the composition is difficult to control. More specifically, a range of fatty acids may be converted to acyl amino acids, even though production of a single adduct may be desirable. Since the mixture comprises compounds highly related in terms of chemical structure, purifying or at least enriching a single component in an efficient and straightforward manner is usually beyond technical feasibility.

Accordingly, there is a need in the art for an efficient and effective biotechnological method of producing acyl amino acids.

### DESCRIPTION OF THE INVENTION

The present invention attempts to solve the problems above by providing at least one method of producing acyl amino acids using a biotechnological method that may be efficient and specific in producing the desired acyl amino acid. In particular, the acyl amino acids are produced from genetically engineered microorganisms. More in particular, the acyl amino acids are produced by culturing a microorganism that is genetically engineered to express at least one amino acid-N-acyl-transferase and at least one acyl-CoA synthetase and to not express/decrease the expression of at least one enzyme involved in glycine metabolism. In particular, the enzyme involved in glycine metabolism may be selected from the group consisting of enzyme of the glycine cleavage system, glycine hydroxymethyltransferase (GlyA), threonine aldolase (LtaE), threonine dehydrogenase (Tdh), 2-Amino-3-Ketobutyrate CoA-Ligase (Kbl) and Allothreonine dehydrogenase (YdfG). In one example, the acyl amino acid is at least one fatty acyl glycinate.

These problems may be solved by the subject matter of the attached claims.

According to one aspect of the present invention, there is provided a cell for producing acyl amino acids wherein the cell is genetically modified to comprise
at least a first genetic mutation that increases the expression relative to the wild type cell of an amino acid-N-acyl-transferase,
at least a second genetic mutation that increases the expression relative to the wild type cell of an acyl-CoA synthetase, and/or
at least a third genetic mutation that decreases the expression relative to the wild type cell of at least one enzyme selected from the group consisting of an enzyme of the glycine cleavage system, glycine hydroxymethyltransferase (GlyA) threonine aldolase (LtaE) threonine dehydrogenase (Tdh),
2-Amino-3-Ketobutyrate CoA-Ligase (Kbl), and allothreonine dehydrogenase (YdfG).

In particular, the cells and methods according to any aspect of the present invention may provide an efficient biotechnological route towards acyl amino acids. The yield and purity of the product from the cells and methods according to any aspect of the present invention, in terms of catalysts or unwanted by-products, may be improved compared to the processes in the state of the art.

The cells and methods according to any aspect of the present invention may also provide a method for making acyl amino acids, wherein the spectrum of fatty acids converted to acyl amino acids may be broader than the processes known in the state of the art. For example, the cells and methods according to any aspect of the present invention may be suitable for converting short and unsaturated fatty acids to acyl amino acids. The cells and methods according to any aspect of the present invention may also provide a biotechnological method for making acyl amino acids, wherein the length of the acyl residue in the acyl amino acid product may be controlled, for example lauryl may be enriched or prevalent.

The cells and methods according to any aspect of the present invention is based on the surprising finding that the combination of amino acid-N-acyl transferase, an acyl-CoA synthetase together with a decrease in expression of at least one enzyme involved in glycine metabolism may be used to convert a variety of fatty acids to acyl amino acids. In particular, amino acid-N-acyl transferases may exist that can be used to convert short unsaturated fatty acids such as lauroleic acid to an acyl amino acid. More in particular, these amino acid-N-acyl-transferases may be capable of converting short unsaturated fatty acids such as lauroleic acid to a larger yield of an acyl amino acid compared to the methods known in the art. Even more in particular, the composition of acyl amino acids produced in the cell according to any aspect of the present invention may be adjusted by varying specifically the length of fatty acids incorporated into such acyl amino acids. For example, the resultant composition of acyl amino acids produced in the cell may be controlled by introducing into the cell one or more specific acyl-CoA thioesterases or altering the expression of one or more acyl-CoA thioesterases endogenously expressed by the cell. The combination of increasing the expression relative to the wild type cell of an amino acid-N-acyl-transferase and acyl-CoA synthetase, and decreasing the expression relative to the wild type cell of at least one enzyme involved in glycine metabolism may result in a synergistic effect enabling an increase in the yield of acyl amino acid compared to the methods known in the art. In one example, the acyl amino acid formed may be lauroylglycinate.

The term “amino acid-N-acyl transferase”, as used herein, refers to an enzyme capable of catalysing the conversion of acyl-CoA, for example the CoA ester of lauroleic acid, and an amino acid, for example a proteinogenic amino acid, in particular glycine, to an acyl amino acid. Suitable amino acid-N-acyl transferases have been described in the prior art, for example in Waluk, D. P. et al, 2010. In one example, the amino acid-N-acyl transferase used according to any aspect of the present invention may comprises a nucleotide sequence of SEQ ID NO:4 or 5. In particular, the amino acid sequence of amino acid-N-acyl transferase may be selected from the group consisting of NP_001010904.1, NP_659453.3, XP_001147054.1, AAH16789.1, AA073139.1, XP_003275392.1, XP_002755356.1, XP_003920208.1, XP_004051278.1, XP_006147456.1, XP_006214970.1, XP_003801413.1, XP_006189704.1, XP_003993512.1, XP_005862181.1, XP_007092708.1, XP_006772167.1, XP_006091892.1, XP_005660936.1, XP_005911029.1, NP_001178259.1, XP_004016547.1, XP_005954684.1, ELR45061.1, XP_005690354.1, XP_004409352.1, XP_007519553.1, XP_004777729.1, XP_005660935.1, XP_004824058.1, XP_006068141.1, XP_006900486.1, XP_007497585.1, XP_002821801.2, XP_007497583.1, XP_003774260.1, XP_001377648.2, XP_003909843.1, XP_003801448.1, XP_001091958.1, XP_002821798.1, XP_005577840.1, XP_001092197.1, NP_001207423.1, NP_001207425.1, XP_003954287.1, NP_001271595.1, XP_003909848.1, XP_004087850.1, XP_004051279.1, XP_003920209.1, XP_005577835.1, XP_003774402.1, XP_003909846.1, XP_004389401.1, XP_002821802.1, XP_003774401.1, XP_007497581.1, EHH21814.1, XP_003909845.1, XP_005577839.1, XP_003774403.1, XP_001092427.1, XP_003275395.2, NP_542392.2, XP_001147271.1, XP_005577837.1, XP_003826420.1, XP_004051281.1, XP_001147649.2, XP_003826678.1, XP_003909847.1, XP_004682812.1, XP_004682811.1, XP_003734315.1, XP_004715052.1, BAG62195.1, XP_003777804.1, XP_003909849.1, XP_001092316.2, XP_006167891.1, XP_540580.2, XP_001512426.1, EAVV73833.1, XP_003464217.1, XP_007519551.1, XP_003774037.1, XP_005954680.1, XP_003801411.1, NP_803479.1, XP_004437460.1, XP_006875830.1, XP_004328969.1, XP_004264206.1, XP_004683490.1, XP_004777683.1, XP_005954681.1, XP_003480745.1, XP_004777682.1, XP_004878093.1, XP_007519550.1, XP_003421399.1, EHH53167.1, XP_006172214.1, XP_003993453.1, AAI12537.1, XP_006189705.1, Q2KIR7.2, XP_003421465.1, NP_001009648.1, XP_003464328.1, XP_001504745.1, ELV11036.1, XP_005690351.1, XP_005216632.1, EPY77465.1, XP_005690352.1, XP_004016544.1, XP_001498276.2, XP_004264205.1, XP_005690353.1, XP_005954683.1, XP_004667759.1, XP_004479306.1, XP_004645843.1, XP_004016543.1, XP_002928268.1, XP_006091904.1, XP_005331614.1, XP_007196549.1, XP_007092705.1, XP_004620532.1, XP_004869789.1, EHA98800.1, XP_004016545.1, XP_004479307.1, XP_004093105.1, NP_001095518.1, XP_005408101.1, XP_004409350.1, XP_001498290.1, XP_006056693.1, XP_005216639.1, XP_007455745.1, XP_005352049.1, XP_004328970.1, XP_002709220.1, XP_004878092.1, XP_007196553.1, XP_006996816.1, XP_005331615.1, XP_006772157.1, XP_007196552.1, XP_004016546.1, XP_007628721.1, NP_803452.1, XP_004479304.1, DAA21601.1, XP_003920207.1, XP_006091906.1, XP_003464227.1, XP_006091903.1, XP_006189706.1, XP_007455744.1, XP_004585544.1, XP_003801410.1, XP_007124812.1, XP_006900488.1, XP_004777680.1, XP_005907436.1, XP_004389356.1, XP_007124811.1, XP_005660937.1, XP_007628724.1, XP_003513512.1, XP_004437813.1, XP_007628723.1, ERE78858.1, EPQ15380.1, XP_005862178.1, XP_005878672.1, XP_540581.1, XP_002928267.1, XP_004645845.1, EPQ05184.1, XP_003513511.1, XP_006214972.1, XP_007196545.1, XP_007196547.1, XP_006772160.1, XP_003801409.1, NP_001119750.1, XP_003801412.1, XP_006772159.1, EAVV73832.1, XP_006091897.1, XP_006772163.1, XP_006091898.1, XP_005408105.1, XP_006900487.1, XP_003993454.1, XP_003122754.3, XP_007455746.1, XP_005331618.1, XP_004585337.1, XP_005063305.1, XP_006091895.1, XP_006772156.1, XP_004051276.1, XP_004683488.1, NP_666047.1, NP_001013784.2, XP_006996815.1, XP_006996821.1, XP_006091893.1, XP_006173036.1, XP_006214971.1, EPY89845.1, XP_003826423.1, NP_964011.2, XP_007092707.1, XP_005063858.1, BAL43174.1, XP_001161154.2, XP_007124813.1, NP_083826.1 XP_003464239.1, XP_003275394.1, ELK23978.1, XP_004878097.1, XP_004878098.1, XP_004437459.1, XP_004264204.1, XP_004409351.1, XP_005352047.1, Q5RFP0.1, XP_005408107.1, XP_007659164.1, XP_003909852.1, XP_002755355.1, NP_001126806.1, AAP92593.1, NP_001244199.1, BAA34427.1, XP_005063859.1, NP_599157.2, XP_004667761.1, XP_006900489.1, XP_006215013.1, XP_005408100.1, XP_007628718.1, XP_003514769.1, XP_006160935.1, XP_004683489.1, XP_003464329.1, XP_004921258.1, XP_003801447.1, XP_006167892.1, XP_004921305.1, AAH89619.1, XP_004706162.1, XP_003583243.1, EFB16804.1, XP_006728603.1, EPQ05185.1, XP_002709040.1, XP_006875861.1, XP_005408103.1, XP_004391425.1, EDL41477.1, XP_006772158.1, EGWO6527.1, AAH15294.1, XP_006772162.1, XP_005660939.1, XP_005352050.1, XP_006091901.1, XP_005878675.1, XP_004051323.1, EHA98803.1, XP_003779925.1, EDM12924.1, XP_003421400.1, XP_006160939.1, XP_006160938.1, XP_006160937.1, XP_006160936.1, XP_005702185.1, XP_005313023.1, XP_003769190.1, XP_002714424.1, XP_004715051.1, XP_007661593.1, XP_004590594.1, ELK23975.1, XP_004674085.1, XP_004780477.1, XP_006231186.1, XP_003803573.1, XP_004803176.1, EFB16803.1, XP_006056694.1, XP_005441626.1, XP_005318647.1 XP_004605904.1, XP_005862182.1, XP_003430682.1, XP_004780478.1, XP_005239278.1, XP_003897760.1, XP_007484121.1, XP_004892683.1, XP_004414286.1, XP_006927013.1, XP_003923145.1, XP_852587.2, AAP97178.1, EHH53105.1, XP_005408113.1, XP_002915474.1, XP_005377590.1, XP_527404.2, XP_005552830.1, XP_004044211.1, NP_001180996.1, XP_003513513.2, XP_001498599.2, XP_002746654.1, XP_005072349.1, XP_006149181.1, EAX04334.1, XP_003833230.1, XP_005216635.1, XP_003404197.1, XP_007523363.1, XP_007433902.1, XP_003254235.1, XP_004471242.1, XP_005216634.1, XP_006860675.1, XP_004771956.1, XP_006038833.1, NP_001138534.1, XP_007068532.1, XP_003510714.1, ERE87950.1, XP_003986313.1, XP_006728644.1, XP_004878099.1, XP_003468014.1, XP_007095614.1, XP_004648849.1, XP_004869795.1, XP_004018927.1, XP_005696454.1, XP_006201985.1, XP_005960697.1, XP_004813725.1, XP_005496926.1, ELR45088.1, XP_004696625.1, XP_005860982.1, XP_005911003.1, XP_006260162.1, EPQ04414.1, XP_006099775.1, NP_001138532.1, XP_006190795.1, XP_004649775.1, XP_004424497.1, XP_004390885.1, XP_005911004.1, XP_003777803.1, XP_004312259.1, XP_005529140.1, XP_005314582.1, XP_006926523.1, XP_006926522.1, XP_004683491.1, XP_003826680.1, XP_003215018.1, XP_003215087.1, EGW12611.1, XP_006113023.1, XP_006882182.1, XP_007425200.1, XP_006041342.1, NP_001138533.1, EMP27694.1, XP_007497753.1, XP_006034252.1 and a variant thereof. The term ‘variant’ as used herein in conjunction with amino acid-N-acyl transferase refers to polypeptides having essentially the amino acid sequence of at least one amino acid-N-acyl transferase mentioned above, but wherein one or more insertions, deletions, additions and/or substitutions intentionally have been made by conventional methods. The variants may vary in the sequence compared to any one of the amino acid-N-acyl transferases mentioned above but they are capable of maintaining their function. In one example, the variant comprises 50, 60, 65, 70, 80, 85, 90, 92, 94, 95, 97, 98, 99% sequence identity in relation to any one of the amino acid-N-acyl transferases mentioned above. In particular, every variant of amino acid-N-acyl transferase is capable of maintaining the function of amino acid-N-acyl transferases. In particular, the amino acid-N-acyl transferase used according to any aspect of the present invention may comprise 50, 60, 65, 70, 80, 85, 90, 92, 94, 95, 97, 98, 99% sequence identity to SEQ ID NO: 63 and 64.

According to any aspect of the present invention, the term “variant” refers to polynucleotides and/or polypeptides that comprise differences in amino acids other than those essential for the function. For example, a variant of a specific enzyme comprises the sequences/parts that result in the catalytic activity of the enzyme, or the fold or structure of the enzyme. The only variations are in the not essential parts of the enzyme. These not so essential amino acids may be deleted, substituted or replaced by insertions or essential amino acids may be replaced in a conservative manner to the effect that the biological activity of the reference sequence or a molecule derived therefrom is preserved. The state of the art comprises algorithms that may be used to align two given nucleic acid or amino acid sequences and to calculate the degree of identity, for example in see Lesk, 2008, Thompson et al., 1994, and Katoh et al., 2005. The term “variant” is used synonymously and interchangeably with the term “homologue”. Such variants may be prepared by introducing deletions, insertions or substitutions in amino acid or nucleic acid sequences as well as fusions comprising such macromolecules or variants thereof. In one example, the term “variant”, with regard to amino acid sequence, comprises, in addition to the sequence identity, amino acid sequences that comprise one or more conservative amino acid changes with respect to the respective reference or wild type sequence or comprises nucleic acid sequences encoding amino acid sequences that comprise one or more conservative amino acid changes. In another example, the term “variant” of an amino acid sequence or nucleic acid sequence comprises, in addition to the degree of sequence identity, any active portion and/or fragment of the amino acid sequence or nucleic acid sequence, respectively, or any nucleic acid sequence encoding an active portion and/or fragment of an amino acid sequence. In a preferred embodiment, the term “active portion”, as used herein, refers to an amino acid sequence or a nucleic acid sequence, which is less than the full length amino acid sequence or codes for less than the full length amino acid sequence, respectively, wherein the amino acid sequence or the amino acid sequence encoded, respectively retains at least some of its essential biological activity. For example an active portion and/or fragment of a protease are capable of hydrolysing peptide bonds in polypeptides. In one example, the term “retains at least some of its essential biological activity”, as used herein, means that the amino acid sequence in question has a biological activity exceeding and distinct from the background activity and the kinetic parameters characterising said activity, more specifically k_{cat} and K_{M}, are within 3, 2, or 1 order of magnitude of the values displayed by the reference molecule with respect to a specific substrate. In one example, the term “variant” of a nucleic acid comprises nucleic acids the complementary strand of which hybridises, under stringent conditions, to the reference or wild type nucleic acid. Examples of variants of amino acid-N-acyl-transferases may at least be provided in FIG. 3. Stringency of hybridisation reactions may be readily determinable by one of ordinary skilled in the art, and in general is an empirical calculation dependent on probe length, washing temperature and salt concentration. In general, longer probes require higher temperatures for proper annealing, while shorter probes need lower temperatures. Hybridisation generally depends on the ability of denatured DNA to reanneal to complementary strands are present in an environment below their melting temperature. The higher the degree of desired homology between the probe and hybridisable sequence, the higher the relative temperature which may be used. As a result it follows that higher relative temperatures would tend to make the reaction conditions more stringent, while lower temperature less so. For additional details and explanation of stringency of hybridisation reactions, see F. M. Ausubel (1995). The person skilled in the art may follow the instructions provided in the manual “The DIG System Users Guide for Filter Hybridization”, 1993 and in Liebl et al. (1991) on how to identify DNA sequences by means of hybridisation.

Stringent conditions may be applied for any hybridisation, i.e. hybridisation occurs only if the probe is 70% or more identical to the target sequence. Probes having a lower degree of identity with respect to the target sequence may hybridise, but such hybrids are unstable and will be removed in a washing step under stringent conditions, for example lowering the concentration of salt to 2×SSC or, optionally and subsequently, to 0.5×SSC, while the temperature is, in order of increasing preference, approximately 50° C.-68° C., approximately 52° C.-68° C., approximately 54° C.-68° C., approximately 56° C.-68° C., approximately 58° C.-68° C., approximately 60° C.-68° C., approximately 62° C.-68° C., approximately 64° C.-68° C., approximately 66° C.-68° C. In one example, the temperature may be approximately 64° C.-68° C. or approximately 66° C.-68° C. It may be possible to adjust the concentration of salt to 0.2×SSC or even 0.1×SSC. Polynucleotide fragments having a degree of identity with respect to the reference or wild type sequence of at least 70, 80, 90, 91, 92, 93, 94, 95, 96, 97, 98, 99% may be isolated. In one example, the term “homologue” of a nucleic acid sequence, as used herein, refers to any nucleic acid sequence that encodes the same amino acid sequence as the reference nucleic acid sequence, in line with the degeneracy of the genetic code.

A skilled person would be able to easily determine the amino acid-N-acyl-transferases that will be capable of making proteinogenic amino acids and/or fatty acids. In particular, the variants may include but are not limited to an amino acid-N-acyl-transferase from any organism selected from the group of organisms consisting of Nomascus leucogenys (NI, XP_003275392.1), Saimiri boliviensis (Sb, XP_003920208.1), Felis catus (Fc, XP_003993512.1), Bos taurus (Bt, NP_001178259.1), and Mus musculus (Mm, NP_666047.1).

The term “acyl amino acid”, as used herein, refers to the product of the reaction catalysed by an amino acid-N-acyl transferase, in particular, a compound represented by the formula acyl-CO—NH—CHR—COOH, wherein R is the side chain of a proteinogenic amino acid, and wherein the term “acyl” refers to the acyl residue of a fatty acid. According to any aspect of the present invention, the term “fatty acid”, as used herein, means a carboxylic acid. In one example, the fatty acid comprises 6, 7, 8, 9, 10, 11, 12, 13, 14, 15, 16, 17, 18, 19, 20, 21, 22, 23, 24, 25, 26, 27, 28, 29, 30 carbon atoms. In particular, the fatty acid may be an alkanoic acid, with at least 6, 8, 10, or 12 carbon atoms. More in particular, the fatty acid has 12 carbon atoms. In one example, a linear fatty acid may be used. In another example, the fatty acid may be branched. In one example, the fatty acid used according to any aspect of the present invention may be a saturated fatty acid. In another example, the fatty acid may be unsaturated. In particular, the fatty acid used according to any aspect of the present invention may be a linear fatty acid with at least 12 carbon atoms comprising a double bond, for example at position 9. In a further example, the fatty acid may be a simple unsaturated fatty acid having one double bond, where the double bond may be located at position 9 or 11. More in particular, the fatty acid used according to any aspect of the present invention may be lauroleic acid (9-dodecenoic acid).

In one example, the acyl amino acid may be a fatty acyl glycinate. In this example, any fatty acid may be used in conjunction with the amino acid glycine. In particular, the fatty acid comprises 6, 7, 8, 9, 10, 11, 12, 13, 14, 15, 16, 17, 18, 19, 20, 21, 22, 23, 24, 25, 26, 27, 28, 29, 30 carbon atoms. More in particular, the fatty acyl glycinate may be any glycinate selected from C6-C24 acyl glycinate. For example, the glycinate may be C6 acyl glycinate, C7 acyl glycinate, C8 acyl glycinate, C9 acyl glycinate, C10 acyl glycinate, C11 acyl glycinate, C12 acyl glycinate, C13 acyl glycinate, C14 acyl glycinate, C15 acyl glycinate, acyl glycinate, C17 acyl glycinate, Cis acyl glycinate, Cis acyl glycinate, C20 acyl glycinate, C21 acyl glycinate, C22 acyl glycinate, C23 acyl glycinate, C24 acyl glycinate or the like.

According to any aspect of the present invention, a formula referring to a chemical group that represents the dissociated or undissociated state of a compound capable of dissociating in an aqueous solution, comprises both the dissociated and the undissociated state and the various salt forms of the group. For example, the residue —COOH comprises both the protonated (—COOH) as well as the unprotonated (—COO⁻) carboxylic acid.

The acyl-CoA substrate used according to any aspect of the present invention may be purified from at least one cell, chemically synthesised and/or produced using an acyl-CoA synthetase. In particular, the acyl-CoA substrate used according to any aspect of the present invention may be produced using an acyl-CoA synthetase.

The term “acyl-CoA synthetase”, as used herein, refers to an enzyme capable of catalysing the ATP-dependent conversion of a fatty acid and CoA to acyl CoA. In particular, acyl-CoA synthetase may refer to an acyl-CoA/ACP synthetase that may be capable of producing acyl glycinates and/or catalysing the following reaction:

fatty acid+CoA/ACP+ATP→acyl-CoA/ACP+ADP+Pi

Examples of acyl-CoA/ACP synthetases may include but are not limited to EC 6.2.1.3, EC 6.2.1.10, EC 6.2.1.15, EC 6.2.1.20 and the like. Acyl-CoA synthetases used according to any aspect of the present invention may be selected from the group consisting of YP_001724804.1, WP_001563489.1, NP_707317.1 and the like. In one example, the acyl-CoA synthetase may comprise SEQ ID NO:65 or YP_001724804.1 or a variant thereof. The term ‘variant’ as used herein in conjunction with Acyl-CoA synthetase refers to polypeptides having essentially the amino acid sequence of at least one Acyl-CoA synthetase mentioned above, but wherein one or more insertions, deletions, additions and/or substitutions intentionally have been made by conventional methods. The variants may vary in the sequence compared to any one of the Acyl-CoA synthetase mentioned above but they are capable of maintaining their function. In one example, the variant comprises 50, 60, 65, 70, 80, 85, 90, 92, 94, 95, 97, 98, 99% sequence identity in relation to any one of the Acyl-CoA synthetases mentioned above.

Various methods to detect acyl-CoA synthetase activity are known in the art. For example, the activity of an acyl-CoA synthetase may be assayed by incubating the sample of interest in 100 mM Tris-HCl at pH 8 in the presence of 250 μM lauroyl-CoA, 500 μM glycine and DTNB (5,5′-dithiobis-2-nitrobenzoic acid, also referred to as Ellman's reagent) and spectrophotometrically monitoring the absorbance at 410 nm following release of free thiol groups in the form of CoASH as the reaction progresses and reaction with Ellman's reagent. The activity of an acyl-CoA synthetase may be assayed as described in the state of the art, for example Kang, Y., 2010. Briefly, the amount of free thiol in the form of unreacted CoASH is determined by adding Ellmann's reagent and spectrophotometrically monitoring the absorbance at 410 nm, preferably in a reaction buffer comprising 150 mM Tris-HCl (pH 7.2), 10 mM MgCl₂, 2 mM EDTA, 0.1% Triton X-100, 5 mM ATP, 0.5 mM coenzyme A (CoASH) and a fatty acid (30 to 300 mM).

In one example, the cell according to any aspect of the present invention may be genetically modified to increase the expression of at least the enzymes amino acid-N-acyl transferase and acyl-CoA synthetase. In particular, the cell may overexpress enzymes glycine N-acyl transferase and acyl-CoA/ACP synthetase. The phrase “increased activity of an enzyme” and “overexpress an enzyme”, as used herein is to be understood as increased intracellular activity. Basically, an increase in enzymatic activity can be achieved by increasing the copy number of the gene sequence or gene sequences that code for the enzyme, using a strong promoter or employing a gene or allele that code for a corresponding enzyme with increased activity and optionally by combining these measures. Genetically modified cells used according to any aspect of the present invention are for example produced by transformation, transduction, conjugation or a combination of these methods with a vector that contains the desired gene, an allele of this gene or parts thereof and a vector that makes expression of the gene possible. Heterologous expression is in particular achieved by integration of the gene or of the alleles in the chromosome of the cell or an extra-chromosomally replicating vector.

The term ‘an enzyme involved in glycine metabolism’ as used herein refers to at least one enzyme that may be capable of reducing the total amount of glycine in a cell by catalysing the breakdown of glycine and/or catalysing the conversion of glycine to other amino acids. In one example, the glycine may be broken down to carbon dioxide and/or ammonia. In another example, the glycine may be converted to serine and/or threonine. In a further example, the glycine may be metabolised in a cell by being broken down to carbon dioxide and/or ammonia and converted to serine and/or threonine simultaneously. FIG. 7 shows a summary of the possible glycine metabolism pathways. In particular, an enzyme involved in glycine metabolism may be selected from the group consisting of at least one enzyme of the glycine cleavage system, glycine hydroxymethyltransferase (GlyA), threonine aldolase (LtaE), threonine dehydrogenase (Tdh), 2-Amino-3-Ketobutyrate CoA-Ligase (Kbl) and Allothreonine dehydrogenase (YdfG).

The term ‘glycine hydroxymethyltransferase’ according to any aspect of the present invention may be directed to an enzyme that catalyses the chemical reaction

5,10-methylenetetrahydrofolate+glycine+H₂O⇄tetrahydrofolate+L-serine

The glycine hydroxymethyltransferase (EC 2.1.2.1) belongs to the family of transferases that transfer one-carbon groups, specifically the hydroxymethyl-, formyl- and related transferases. In particular, GlyA used according to any aspect of the present invention may comprise a polypeptide sequence of SEQ ID NO:61 or variants thereof. The term ‘variant’ as used herein in conjunction with GlyA refers to polypeptides having essentially the same sequence as SEQ ID NO:61 but wherein one or more insertions, deletions, additions and/or substitutions intentionally have been made by conventional methods. The variants may vary in the sequence compared to SEQ ID NO:61 but they are capable of maintaining their function. In one example, the variant comprises 50, 60, 65, 70, 80, 85, 90, 92, 94, 95, 97, 98, 99% sequence identity in relation to SEQ ID NO:61. Similarly, LtaE used according to any aspect of the present invention may comprise a polypeptide sequence of SEQ ID NO:62 or variants thereof. The term ‘variant’ as used herein in conjunction with LtaE refers to polypeptides having essentially the same sequence as SEQ ID NO:62 but wherein one or more insertions, deletions, additions and/or substitutions intentionally have been made by conventional methods. The variants may vary in the sequence compared to SEQ ID NO:62 but they are capable of maintaining their function. In particular, the variant of LtaE may comprise 50, 60, 65, 70, 80, 85, 90, 92, 94, 95, 97, 98, 99% sequence identity in relation to SEQ ID NO:62. Similarly, Tdh used according to any aspect of the present invention may comprise a polypeptide sequence of SEQ ID NO:81 or variants thereof. The term ‘variant’ as used herein in conjunction with Tdh refers to polypeptides having essentially the same sequence as SEQ ID NO:81 but wherein one or more insertions, deletions, additions and/or substitutions intentionally have been made by conventional methods. The variants may vary in the sequence compared to SEQ ID NO:81 but they are capable of maintaining their function. In particular, the variant of Tdh may comprise 50, 60, 65, 70, 80, 85, 90, 92, 94, 95, 97, 98, 99% sequence identity in relation to SEQ ID NO:81. Kbl used according to any aspect of the present invention may comprise a polypeptide sequence of SEQ ID NO:80 or variants thereof. The term ‘variant’ as used herein in conjunction with Kbl refers to polypeptides having essentially the same sequence as SEQ ID NO:80 but wherein one or more insertions, deletions, additions and/or substitutions intentionally have been made by conventional methods. The variants may vary in the sequence compared to SEQ ID NO:80 but they are capable of maintaining their function. In particular, the variant of Kbl may comprise 50, 60, 65, 70, 80, 85, 90, 92, 94, 95, 97, 98, 99% sequence identity in relation to SEQ ID NO:80. Also, YdfG used according to any aspect of the present invention may comprise a polypeptide sequence of SEQ ID NO:82 or variants thereof. The term ‘variant’ as used herein in conjunction with Kbl refers to polypeptides having essentially the same sequence as SEQ ID NO:82 but wherein one or more insertions, deletions, additions and/or substitutions intentionally have been made by conventional methods. The variants may vary in the sequence compared to SEQ ID NO:82 but they are capable of maintaining their function. In particular, the variant of YdfG may comprise 50, 60, 65, 70, 80, 85, 90, 92, 94, 95, 97, 98, 99% sequence identity in relation to SEQ ID NO:82.

The phrase “at least one enzyme from the glycine cleavage system” refers to a series of enzymes that are triggered in response to high concentrations of the amino acid glycine. The glycine cleavage system is composed of four proteins: the T-protein (GcvT), P-protein (GcvP), L-protein (GcvL), and H-protein (GcvH). The H-protein is responsible for interacting with the three other proteins and acts as a shuttle for some of the intermediate products in glycine decarboxylation. In both animals and plants the glycine cleavage system is loosely attached to the inner membrane of the mitochondria. The expression of any one of these enzymes may be decreased according to any aspect of the present invention. More in particular, the glycine cleavage system H protein, carries lipoic acid and interacts with the glycine cleavage system proteins P, L and T; the glycine cleavage system P protein (EC 1.4.4.2), catalyses the reaction glycine+[glycine-cleavage complex H protein]-N(6)-lipoyl-L-lysine→[glycine-cleavage complex H protein]-S-aminomethyl-N(6)-dihydrolipoyl-L-lysine+CO2; glycine cleavage system L protein (EC 1.8.1.4), catalyses the reaction protein N6-(dihydrolipoyl)lysine+NAD+→protein N6-(lipoyl)lysine+NADH+H+; glycine cleavage system T protein (EC 2.1.2.10), catalyses the reaction [protein]-S8-aminomethyldihydrolipoyllysine+tetrahydrofolate→[protein]-dihydrolipoyllysine+5,10-methylenetetrahydrofolate+NH₃; threonine aldolase (EC 4.2.1.48), catalyses the reaction L-threonine→glycine+acetaldehyde; serine hydroxylmethyltransferase (EC 2.1.2.1), catalyses the reaction 5,10-methylenetetrahydrofolate+glycine+H₂O+tetrahydrofolate+L-serine.

Enzymes from the glycine cleavage system used according to any aspect of the present invention may be selected from the sequences mentioned in Table 1 below and variants thereof. The term ‘variant’ as used herein in conjunction with an enzyme from the glycine cleavage system refers to polypeptides having essentially the amino acid sequence of at least one enzyme from the glycine cleavage system mentioned in Table 1, but wherein one or more insertions, deletions, additions and/or substitutions intentionally have been made by conventional methods. The variants may vary in the sequence compared to any one of the enzyme from the glycine cleavage system mentioned below but they are capable of maintaining their function. In one example, the variant comprises 50, 60, 65, 70, 80, 85, 90, 92, 94, 95, 96, 97, 98 or 99% sequence identity in relation to any one of the enzyme from the glycine cleavage system mentioned below. More in particular, the sequence of GcvT may be SEQ ID NO:58. The sequence of GcvP may be SEQ ID NO:60. The sequence of GcvH may be SEQ ID NO:59. Even more in particular the sequences of GcvT, GcvP and GcvP may comprise SEQ ID NO:58, SEQ ID NO:60 and SEQ ID NO:59 respectively. Throughout this application, any data base code, unless specified to the contrary, refers to a sequence available from the NCBI data bases, more specifically the version online on 6 Aug. 2014, and comprises, if such sequence is a nucleotide sequence, the polypeptide sequence obtained by translating the former.

TABLE 1
Sequences of enzymes that are part of the Gcv system.

| Enzyme | Accession Number |
|---|---|
| GcvT | WP_000068701; CAA52144.1, YP_079782.1; NP_980596.1; YP_021091.1; |
| | AA11139.1; YP_148276.1; YP_175989.1; BAB06533.1; NP_464875.1; |
| | NP_470723.1; YP_013965.1; ZP_00231385.1; ZP_00538577.1; NP_692823.1; |
| | NP_764775.1; YP_188676.1; NP_372059.1; CAG43268.1; YP_253296.1; |
| | YP_186433.1; YP_041008.1; NP_621985.1; ZP_00560257.1; AAU84894.1; |
| | ZP_00574838.1; YP_075748.1; NP_143816.1; CAB50682.1; NP_662997.1; |
| | NP_972230.1; AAL82124.1; ZP_00590815.1; ZP_00528533.1; BAD85568.1; |
| | NP_228029.1; ZP_00511104.1; ZP_00591209.1; ZP_00532593.1; |
| | ZP_00525162.1; ZP_00355911.1; YP_112880.1; ZP_00399764.1; YP_143792.1; |
| | CAD75448.1; YP_004126.1; ZP_00535960.1; ZP_00334892.1; CAG35027.1; |
| | CAF23008.1; YP_010643.1; NP_951437.1; NP_840694.1; EAN28088.1; |
| | YP_125490.1; YP_094168.1; AAO91208.1; YP_122478.1; NP_110817.1; |
| | NP_214308.1; CAC12478.1; ZP_00602311.1; YP_256007.1; EAM94548.1; |
| | BAB66249.1; NP_342409.1; YP_023949.1; ZP_00054699.1; AAK25314.1; |
| | ZP_00270640.1; YP_169455.1; NP_148400.1; ZP_00577149.1; ZP_00375766.1; |
| | ZP_00303628.1; NP_280387.1; AAV46419.1; EAO04791.1; CAC32302.1; |
| | CAI36361.1; AAZ12256.1; NP_013914.1; YP_034020.1; BAC74698.1; Q827D7; |
| | ZP_00396527.1; EAK92665.1; EAK92694.1; ZP_00292858.1; ZP_00625542.1; |
| | NP_298674.1; EAO14274.1; NP_883104.1; NP_887405.1; ZP_00038971.2; |
| | NP_778843.1; ZP_00327636.1; EAA72140.1; NP_879086.1; NP_102591.1; |
| | CAJ04455.1; CAG61762.1; NP_716412.1; CAG88846.1; ZP_00582521.1; |
| | CAD17083.1; AAM36086.1; YP_191522.1; EAM75690.1; CAH00726.1; |
| | CAF26479.1; AAF11360.1; YP_202186.1; ZP_00308932.1; EAA47849.1; |
| | ZP_00278041.1; CAA81076.1; CAA91000.1; CAA85353.1; EAM85167.1; |
| | YP_071681.1; ZP_00638529.1; CAB16911.1; EAN06353.1; BAD35509.1; |
| | EAL84525.1; YP_118701.1; AAQ24377.1; YP_223286.1; NP_541539.1; |
| | ZP_00634544.1; CAB16916.1; BAD82264.1; AAN47559.1; NP_772393.1; |
| | YP_000299.1; CAB16918.1; CAD52982.1; XP_314216.2; NP_930808.1; |
| | AAD56281.1; AAQ66378.1; AAO76254.1; AAN33907.1; CAG73659.1; |
| | XP_219785.3; ZP_00111607.1; ZP_00585062.1; AAL21928.1; AAH42245.1; |
| | AAX66900.1; AAS46734.1; NP_949187.1; NP_989653.1; AAC31228.1;; |
| | AAA63798.1; ZP_00004510.1; CAA42443.1; ZP_00555139.1; YP_132995.1; |
| | NP_251135.1; NP_636487.1; ZP_00213263.1; ZP_00140178.2; ZP_00516950.1; |
| | AAB82711.1; AAZ27773.1; EAA61388.1; ZP_00167208.2; EAN04068.1; |
| | AAO07159.1; NP_936747.1; ZP_00499479.1; NP_806663.1; AAW42121.1; |
| | CAG83849.1; NP_923192.1; NP_441838.1; ZP_00593912.1; CAA52146.1; |
| | BAA14286.1; Q8XD33; AAA69071.1; EAM28058.1; ZP_00494650.1; |
| | ZP_00489316.1; ZP_00470629.1; YP_152074.1; AAM14125.1; AAM91322.1; |
| | ZP_00569907.1; ZP_00459442.1; NP_755358.1; ZP_00436271.1; NP_613061.1; |
| | CAE59244.1; BAC38022.1; CAA91099.1; ZP_00264533.1; AAC46780.1; |
| | AAG58030.1; YP_261725.1; AAL24244.1; NP_708666.1; CAH07776.1; Q8YNF9; |
| | YP_099306.1; ZP_00474391.1; AAL57651.1; YP_055456.1; NP_682393.1; |
| | NP_893785.1; YP_172756.1; CAH74116.1; ZP_00622638.1; YP_234188.1; |
| | NP_791106.1; NP_967658.1; EAN85656.1; ZP_00379711.1; XP_520482.1; |
| | AAN66613.1; ZP_00162707.1; ZP_00417692.1; YP_264083.1; AAQ61092.1; |
| | NP_216348.1; NP_855515.1; CAE76410.1; AAO39460.1; CAE22343.1; |
| | NP_800311.1; YP_206661.1; ZP_00506636.1; ZP_00417032.1; YP_164890.1; |
| | XP_760322.1; YP_266091.1; YP_156473.1; CAC46126.1; ZP_00244924.1; |
| | EAN91112.1; XP_637330.1; NP_960479.1; YP_160522.1; ZP_00317484.1; |
| | ZP_00412767.1; NP_253900.1; AAQ00873.1; NP_532152.1; ZP_00151464.1; |
| | EAO09970.1; ZP_00631036.1; ZP_00141690.2; NP_302381.1; CAG08109.1; |
| | CAE08889.1; YP_263017.1; AAN70757.1; ZP_00264788.1; XP_538655.1; |
| | AAZ27305.1; AAN17423.1; AAO38610.1; CAB85154.1; AAW89976.1; |
| | AAO44232.1; NP_789087.1; ZP_00496567.1; AAH57478.1; AAS16361.1; |
| | AAL33595.1; ZP_00048418.1; CAA72255.1; XP_598207.1; BAD82265.1; |
| | AAK26613.1; AAM93931.1; XP_517277.1; CAB50683.1; NP_143817.1; |
| | AAL82123.1; BAD82266.1; EAN80863.1; BAD85569.1; ZP_00574837.1; |
| | BAB26854.1; ZP_00560256.1; AAU84893.1; AAD33990.1; YP_175990.1; |
| | ZP_00641578.1; NP_390336.1; YP_023948.1; AAK25315.1; CAA38252.1; |
| | EAM94547.1; ZP_00530977.1; YP_013964.1; NP_470722.1; NP_464874.1; |
| | ZP_00525161.1; NP_148401.1; ZP_00589915.1; YP_079783.1; ZP_00233535.1; |
| | ZP_00577150.1; NP_228028.1; ZP_00238490.1; NP_980597.1; YP_021092.1; |
| | AAP11140.1; YP_038289.1; BAB06534.1; ZP_00399763.1; YP_148277.1; |
| | XP_606427.1; AAL04442.1; YP_075749.1; ZP_00527784.1; NP_692824.1; |
| | ZP_00334895.1; CAD75449.1; NP_840693.1; ZP_00591425.1; CAF23007.1; |
| | ZP_00054162.1; NP_621986.1; CAC12479.1; NP_662508.1; ZP_00512041.1; |
| | AAV46420.1; ZP_00375765.1; NP_110816.1; NP_280388.1; BAB66248.1; |
| | ZP_00538578.1; NP_951436.1; ZP_00367500.1; YP_169454.1; AAX77943.1; |
| | YP_094170.1; YP_122480.1; YP_125492.1;; ZP_00270641.1; YP_178313.1; |
| | XP_473181.1; ZP_00535961.1; NP_372060.1; ZP_00602310.1; YP_255986.1; |
| | CAE05443.2; CAB72709.1; CAG43269.1; NP_342410.1; BAB95353.1; |
| | AAA92036.1; YP_186434.1; YP_041009.1; AAM63785.1; XP_584346.1; |
| | AAM51302.1; AAF99780.1; ZP_00563258.1; EAN09988.1; NP_148290.1; |
| | YP_253295.1; ZP_00627278.1; YP_256008.1; AAO91209.1; XP_655257.1; |
| | ZP_00303629.1; CAC11669.1; NP_394004.1; EAM93962.1; CAB57769.1; |
| | CAB50124.1; ZP_00520055.1; ZP_00369709.1; EAN28089.1; CAC12627.1; |
| | AAB85605.1; Q9HI38; AAH26135.1; EAM24571.1; AAC03768.1; AAP98645.1; |
| | CAJ18422.1; NP_213757.1; CAA05909.1; NP_142859.1; BAD32415.1; |
| | BAC31437.1; NP_947805.1; YP_080623.1; CAH93356.1; ZP_00600159.1; |
| | NP_775139.1; ZP_00400734.1; CAA09590.2; XP_321361.2; AAL81283.1; |
| | BAB81491.1; ZP_00506445.1; ZP_00533640.1; YP_160688.1; NP_764776.1; |
| | NP_967201.1; AAH52991.1; AAO38289.1; NP_837365.1; XP_521504.1; |
| | NP_371248.1; NP_753970.1; CAG42465.1; AAC74750.1; AAU93791.1; Q7ADI4; |
| | 1KMK; 1JF9; BAA86566.1; YP_170184.1; YP_165179.1; NP_624289.1; |
| | AAX77932.1; CAD14721.1; BAD84717.1; ZP_00048472.2; AAF73526.1; |
| | XP_641773.1; ZP_00570672.1; ZP_00400089.1; NP_391148.1; NP_001007947.1; |
| | CAE08806.1; CAB50330.1; YP_192068.1; ZP_00152262.1; ZP_00502920.1; |
| | YP_096188.1; NP_796975.1; YP_254084.1; YP_124440.1; BAD86003.1; |
| | CAH04407.1; BAB07188.1; Q9K7A0; XP_313472.2; NP_103175.1; AAF63783.1; |
| | AAN58019.1; YP_127437.1; AAM62682.1; CAG90500.1; AAB85857.1; |
| | ZP_00387883.1; XP_562557.1; O27433; ZP_00170920.2; CAA63066.1; |
| | EAN08419.1; CAB73027.1; ZP_00335679.1; AAB85866.1; ZP_00129032.1; |
| | YP_178855.1; YP_145876.1; AAV94639.1; NP_623991.1; YP_040300.1; |
| | YP_040204.1; YP_080545.1; YP_253952.1; ZP_00638523.1; NP_371368.1; |
| | YP_255707.1; AAU38404.1; NP_085678.1; AAK68403.2; BAB66416.1; |
| | XP_624944.1; ZP_00135005.2; ZP_00595047.1; NP_950100.1; ZP_00458730.1; |
| | AAQ75173.1; AAP99129.1; YP_004062.1; XP_601722.1; CAC45089.1; |
| | CAA07007.1; EAL01528.1; XP_694140.1; NP_770978.1; AAC49935.1; |
| | EAA21518.1 |
| GcvH | WP_001295377; NP_391159; YP_152075.1; NP_806664.1; NP_755359.1; |
| | Q8FE66; AAX66901.1; ZP_00585063.1; NP_716411.1; ZP_00582520.1; |
| | NP_930809.1; YP_071682.1; ZP_00638530.1; CAG73658.1; YP_156474.1; |
| | ZP_00634545.1; YP_131233.1; YP_268018.1; ZP_00417033.1; AAN70758.1; |
| | ZP_00141691.2; NP_253901.1; AAZ18651.1; EAO18275.1; ZP_00264789.1; |
| | ZP_00474390.1; ZP_00499480.1; ZP_00459441.1; EAM28059.1; ZP_00318113.1; |
| | AAQ61093.1; NP_790167.1; AAO91210.1; CAH37374.1; YP_169453.1; |
| | AAW49868.1; NP_840692.1; AAO07160.1; YP_263019.1; Q9K0L7; YP_160523.1; |
| | NP_800312.1; ZP_00334896.1; ZP_00278042.1; YP_233358.1; AAW90049.1; |
| | ZP_00213264.1; AAF96187.1; CAB84042.1; AAM37905.1; ZP_00658478.1; |
| | YP_112591.1; YP_132994.1; YP_200434.1; ZP_00593911.1; YP_206660.1; |
| | ZP_00167207.1; XP_464281.1; NP_638224.1; EAM75684.1; ZP_00574836.1; |
| | ZP_00151463.1; YP_270507.1; CAD17082.1; BAD45416.1; YP_004124.1; |
| | YP_143790.1; YP_075750.1; AAF11361.1; NP_465948.1; CAG35029.1; |
| | NP_960473.1; ZP_00568923.1; EAN06352.1; ZP_00506635.1; NP_532153.1; |
| | YP_223285.1; NP_541538.1; YP_014985.1; AAO63775.1; CAA20174.1; |
| | ZP_00399762.1; YP_010645.1; NP_471849.1; AAO77626.1; YP_094171.1; |
| | NP_693309.1; BAC70485.1; NP_228027.1; YP_253966.1; 1ZKO; YP_148857.1; |
| | YP_046528.1; YP_040289.1; ZP_00131107.1; YP_176481.1; NP_297474.1; |
| | Q9PGW7; ZP_00355907.1; ZP_00651773.1; NP_778393.1; YP_021882.1; |
| | NP_855509.1; CAF26480.1; CAG42548.1; ZP_00389942.1; NP_981423.1; |
| | AAP11863.1; NP_302386.1; NP_764151.1; YP_266090.1; ZP_00237746.1; |
| | ZP_00244923.1; ZP_00396528.1; YP_188077.1; CAC46127.1; ZP_00375764.1; |
| | ZP_00560255.1; ZP_00270642.1; BAB07203.1; EAN28788.1; NP_216342.1; |
| | CAH09835.1; NP_251136.1; YP_185749.1; AAT49691.1; ZP_00264532.1; |
| | NP_102590.1; YP_034021.1; NP_879085.1; AAP54618.1; ZP_00625541.1; |
| | ZP_00525160.1; AAV46421.1; ZP_00412765.1; CAH02746.1; CAA81075.1; |
| | CAB16912.1; CAB16710.1; CAA85761.1; ZP_00622637.1; CAB16914.1; |
| | CAA85759.1; NP_143205.1; YP_101635.1; ZP_00380048.1; ZP_00004511.1; |
| | ZP_00414055.1; AAC61829.1; AAL81616.1; YP_261724.1; CAB49742.1; |
| | AAN66614.1; Q9V0G1; YP_080558.1; BAD84339.1; AAK25316.1; NP_949186.1; |
| | CAJ13836.1; AAQ67414.1; 1DXM; CAA85768.1; CAA85757.1; P93255; |
| | CAJ13723.1; CAA85755.1; ZP_00292299.1; ZP_00308328.1; NP_772392.1; |
| | CAI36363.1; NP_147622.1; NP_391159.1; ZP_00577152.1; CAA85756.1; |
| | AAQ66080.1; AAV94183.1; CAA88734.1; ZP_00547429.1; NP_621789.1; |
| | CAA85760.1; AAR37471.1; ZP_00555140.1; AAW47059.1; ZP_00303630.1; |
| | NP_621987.1; AAG48828.1; CAG86839.1; YP_191521.1; YP_055457.1; |
| | ZP_00631037.1; YP_118696.1; CAA85767.1; XP_637044.1; AAM64413.1; |
| | CAC19751.1; ZP_00379709.1; NP_280389.1; CAF23006.1; EAO25275.1; |
| | AAU84892.1; CAF92157.1; EAN04067.1; AAH91548.1; YP_164889.1; |
| | NP_213756.1; CAG62852.1; AAW49010.1; CAA94317.1; ZP_00527786.1; |
| | YP_234189.1; CAD52976.1; NP_967650.1; CAE66592.1; EAA66192.1; |
| | AAW27708.1; ZP_00565058.1; XP_536768.1; BAB66246.1; CAA95820.1; |
| | AAL68248.1; AAH14745.1; NP_791107.1; EAL90537.1; XP_579628.1; |
| | XP_316586.2; AAP88829.1; NP_004474.2; NP_080848.1; NP_951435.1; |
| | XP_523434.1; CAA85754.1; Q9N121; AAS59848.1; YP_172757.1; |
| | ZP_00534758.1; ZP_00591423.1; ZP_00512042.1; ZP_00661686.1; |
| | NP_883103.1; ZP_00054163.1; NP_598282.1; AAW31875.1; XP_756407.1; |
| | AAH76212.1; CAF99616.1; CAA94316.1; NP_953067.1; EAA72139.1; |
| | NP_110807.1; AAS52315.1; ZP_00575020.1; P20821; NP_662509.1; |
| | CAG33353.1; CAE63163.1; ZP_00589916.1; XP_217678.1; YP_256010.1; |
| | XP_582835.1; CAC12487.1; NP_394822.1; ZP_00535962.1; NP_001004372.1; |
| | EAL29812.1; CAB05472.1; XP_615385.1; EAL03567.1; O22535; AAH82740.1; |
| | AAX07637.1; NP_926477.1; |
| | EAM93557.1; XP_584988.1; AAM92707.1 ZP_00515529.1; ZP_00530979.1; |
| | AAH81062.1; ZP_00111606.1; NP_682468.1; Q8DIB2; EAA77334.1; AAN47560.1; |
| | AAL33596.1; ZP_00162706.2; NP_342412.1; Q8G4Z7; ZP_00120558.2; |
| | ZP_00136571.1; NP_972232.1; CAE18120.1; Q8YNF8; XP_604979.1; |
| | CAE08890.1; CAE76092.1; YP_023402.1; NP_009355.2; P39726; CAD75450.1; |
| | NP_440920.1; CAE47935.1; XP_498178.1; NP_893786.1; ZP_00327635.1; |
| | EAN76953.1; ZP_00140179.2; EAN99694.1; AAZ14696.1; CAE22344.1; |
| | EAN83079.1; BAB26349.1; YP_169819.1; AAX78078.1; AAQ00874.1; |
| | AAC36844.1; CAG78944.1; XP_694123.1; ZP_00050263.1; AAO44734.1; |
| | XP_414165.1; ZP_00654389.1; ZP_00575538.1; XP_518701.1; BAB66989.1; |
| | NP_342534.1; XP_583383.1; NP_213643.1; YP_255059.1; NP_214139.1; |
| | NP_213280.1; ZP_00540302.1; YP_055788.1; AAP05107.1; AAF39393.1; |
| | ZP_00399135.1; NP_701199.1; CAD75020.1; XP_343995.2; AAP98380.1; |
| | P_300490.1; XP_635521.1; YP_219766.1; XP_637059.1; AAV71155.1; |
| | XP_739104.1; EAA18076.1; NP_219787.1; ZP_00400682.1; NP_967270.1; |
| | ZP_00384691.1; ZP_00401280.1; NP_816146.1; ZP_00526092.1; NP_213714.1; |
| | YP_255058.1; NP_785822.1; BAB66988.1; NP_253484.1; ZP_00141248.1; |
| | ZP_00399137.1; XP_676766.1; EAO22790.1; NP_342572.1; AAF07900.1; |
| | ZP_00152140.2; XP_518003.1; AAA23866.1; AAP06383.1; AAM99940.1; |
| | ZP_00523572.1; AAH09065.1; YP_039780.1; NP_735539.1; NP_326272.1; |
| | YP_252181.1; NP_802319.1; XP_637062.1; AAK70873.1; ZP_00523574.1; |
| | NP_784119.1; XP_356748.3; YP_115837.1; XP_520481.1; BAB94166.1; |
| | XP_527720.1; NP_975513.1; ZP_00511802.1; ZP_00660576.1; XP_598843.1; |
| | ZP_00335312.1; ZP_00501149.1; ZP_00496208.1; ZP_00488962.1; |
| | ZP_00481092.1; ZP_00470769.1; YP_112037.1; ZP_00315286.1; |
| | ZP_00591859.1; NP_622848.1; CAB59889.1; ZP_00566435.1; ZP_00633779.1; |
| | AAG13505.2; CAB16915.1; ZP_00585787.1; ZP_00531610.1; AAW51218.1; |
| | YP_263215.1; AAK22373.1; AAT58044.1; EAA20319.1; NP_842410.1; |
| | EAN32517.1; NP_532021.1; AAH75478.1; NP_533976.1; ZP_00385854.1; |
| | AAL04441.1; AAK89915.1; BAD16654.1; NP_216731.1; NP_770578.1; |
| | ZP_00547768.1; NP_522769.1; ZP_00383064.1; ZP_00322497.1; YP_022846.1; |
| | NP_961247.1; YP_238125.1; ZP_00412124.1; XP_475165.1; XP_470945.1; |
| | NP_910410.1; NP_635909.1; CAE01575.2; XP_419793.1; ZP_00170555.1; |
| | NP_217017.1;; ZP_00556997.1; AAP99073.1; NP_297341.1; AAX73221.1; |
| | NP_214109.1; NP_440434.1; CAG87711.1; ZP_00051435.1; BAB80778.1; |
| | XP_393389.2; ZP_00651791.1; ZP_00269419.1; ZP_00423019.1; YP_170419.1; |
| | NP_001006383.1; XP_482561.1; NP_778293.1; CAB03400.1; CAH04871.1; |
| | XP_655127.1; AAF61288.1; ZP_00356683.1; ZP_00565878.1; CAJ01708.1; |
| | XP_758384.1; AAU38115.1; AAK23858.1; XP_688912.1; BAB06344. |
| GcvP | WP_000195062; CAA52144, NP_390336; NP_708668.1; Q8XD32; NP_755360.1; |
| | 1VLO; YP_152076.1; AAX66902.1; YP_071683.1; NP_670593.1; NP_930810.1; |
| | CAG73657.1; ZP_00585064.1; ZP_00634546.1; NP_716410.1; ZP_00638531.1; |
| | ZP_00582519.1; YP_156475.1; YP_268017.1; ZP_00141692.2; ZP_00417034.1; |
| | NP_253902.1; AAT51348.1; ZP_00318114.1; YP_233357.1; AAO91211.1; |
| | AAM37906.1; ZP_00264790.1; NP_790166.1; YP_125494.1; YP_263020.1; |
| | YP_122482.1; YP_094172.1; AAN70759.1; Q5ZZ93; YP_200433.1; NP_638225.1; |
| | YP_112882.1; NP_778394.1; NP_840691.1; EAO18276.1; NP_297476.1; |
| | ZP_00651772.1; YP_160524.1; AAQ61094.1; ZP_00334897.1; YP_104498.1; |
| | ZP_00213265.1; EAM28060.1; NP_887403.1; NP_883102.1; ZP_00151462.1; |
| | ZP_00654390.1; NP_879084.1; ZP_00459440.1; ZP_00454903.1; |
| | ZP_00278043.1; ZP_00499481.1; EAN28090.1; CAD17081.1; Q9K0L8; |
| | AAZ18652.1; CAB84041.1; Q9JVP2; ZP_00167206.1; YP_169452.1; |
| | AAW90051.1; AAW49997.1; ZP_00593910.1; ZP_00419736.1; ZP_00560254.1; |
| | YP_041010.1; YP_186435.1; YP_253294.1; NP_764777.1; YP_079784.1; |
| | ZP_00325013.1; YP_172504.1; BAB06535.1; NP_621988.1; NP_390337.1; |
| | NP_464873.1; ZP_00233534.1; ZP_00165293.2; YP_013963.1; NP_470721.1; |
| | NP_681534.1; BAB76308.1; ZP_00399761.1; ZP_00517920.1; NP_692825.1; |
| | Q8CXD9; NP_926476.1; Q7NFJ5; YP_075751.1; NP_228026.1; ZP_00162705.1; |
| | YP_148278.1; AAQ00895.1; 1YX2; ZP_00355906.1; ZP_00111605.1; |
| | AAU84891.1; NP_441988.1; YP_085561.1; ZP_00238491.1; NP_980598.1; |
| | ZP_00530333.1; NP_662667.1; AAP11141.1; CAE22389.1; CAE08940.1; |
| | EAO25274.1; ZP_00574835.1; ZP_00511544.1; NP_967651.1; YP_004123.1; |
| | ZP_00590889.1; YP_175991.1; ZP_00538579.1; YP_143789.1; ZP_00533333.1; |
| | ZP_00307846.1; ZP_00396529.1; ZP_00660916.1; ZP_00588126.1; |
| | ZP_00525159.1; ZP_00531283.1; NP_893804.1; CAI36362.1; AAO79689; |
| | AAX16385.1; NP_393488.1; CAH07007.1; CAA20175.1; NP_110577.1; |
| | ZP_00292300.1; YP_055458.1; BAB59199.1; BAC70484.1; EAM94419.1; |
| | NP_960885.1; ZP_00412766.1; YP_117906.1; NP_301653.1; NP_972233.1; |
| | NP_295535.1; AAQ66593.1; NP_216727.1; P64220; ZP_00646130.1; |
| | YP_023281.1; CAC11159.1; O67441; EAM73669.1; CAF23005.1; ZP_00656447.1; |
| | AAN47561.1; YP_000301.1; CAD75451.1; Q8F935; AAV46422.1; Q5V230; |
| | ZP_00631038.1; AAO07163.1; ZP_00379710.1; AAK25317.1; ZP_00574284.1; |
| | ZP_00549460.1; AAL33597.1; NP_936752.1; XP_473945.1; CAA81081.1; |
| | ZP_00054164.1; ZP_00577153.1; CAB16917.1; NP_143049.1; O58888; |
| | CAB50008.1; BAD86224.1; AAB38502.1; YP_164888.1; EAL00308.1; |
| | NP_280390.1; YP_206658.1; Q9HPJ7; CAA52800.1; EAL00186.1; |
| | ZP_00130510.2; AAP21169.1; NP_800315.1; CAA81077.1; CAA94902.1; |
| | NP_789581.1; CAA10976.1; AAO44735.1; XP_756577.1; YP_266089.1; |
| | CAB11698.1; ZP_00535963.1; NP_949185.1; XP_629708.1; ZP_00270643.1; |
| | AAK87256.1; AAL81465.1; YP_261727.1; NP_010302.1; EAN06351.1; |
| | YP_010902.1; CAG77727.1; AAB05000.1; ZP_00303631.1; NP_001006021.1; |
| | ZP_00625540.1; CAG85941.1; NP_951434.1; NP_772391.1; CAF93361.1; |
| | CAH02226.1; YP_270503.1; CAF26481.1; ZP_00601921.1; XP_394029.2; |
| | ZP_00474388.1; CAC46128.1; AAN66611.1; ZP_00264535.1; NP_251132.1; |
| | ZP_00375763.1; ZP_00140175.2; ZP_00555141.1; YP_234187.1; AAT51611.1; |
| | ZP_00622636.1; ZP_00506634.1; NP_791105.1; AAR21108.1; ZP_00417689.1; |
| | EAN86200.1; AAW42395.1; EAL33114.1; BAB66247.1; NP_532154.1; |
| | YP_034022.1; NP_102589.1; XP_331926.1; EAN85387.1; CAG58515.1;; |
| | AAB37080.1; ZP_00244922.1; YP_191520.1; CAJ09347.1; CAJ09346.1; |
| | CAE64583.1; AAN33909.1; YP_223284.1; NP_214006.1; BAA02967.1; |
| | BAA03512.1; AAF52996.1; AAX33383.1; XP_322034.2; NP_541537.1; |
| | NP_001013836.1; EAA68431.1; NP_001014026.1; XP_620786.1; EAA51066.1; |
| | EAN04066.1; XP_541886.1; YP_256009.1; Q9TSZ7; YP_132990.1; NP_990119.1; |
| | AAH07546.2; EAA65791.1; Q9YBA2; EAL90405.1; CAC41491.1; NP_342411.1; |
| | EAN79919.1; XP_517018.1; NP_107651.1; ZP_00004512.2; BAA12709.1; |
| | NP_148104.1; AAV94849.1; YP_266710.1; XP_516459.1; NP_105581.1; |
| | ZP_00620069.1; ZP_00631895.1; AAL13520.1; YP_047137.1; AAF68432.3; |
| | CAC46853.1; XP_542460.1; ZP_00460296.1; ZP_00554633.1; ZP_00282393.1; |
| | NP_102909.1; YP_235303.1; AAQ87218.1; ZP_00213445.1; NP_106044.1; |
| | ZP_00169723.2; ZP_00500952.1; ZP_00489849.1; ZP_00480439.1; |
| | ZP_00450235.1; ZP_00436058.1; AAY59105.1; EAM32228.1; AAW21506.1; |
| | CAD14805.1; ZP_00410721.1; NP_792264.1; Q46337; NP_521609.1; |
| | BAD97818.1; 1X31; NP_534554.1; ZP_00602139.1; AAK16489.1; YP_266475.1; |
| | EAA22341.1; XP_318114.2; CAC47432.1; ZP_00004192.1; NP_534790.1; |
| | AAL52901.1; ZP_00565350.1; ZP_00657316.1; YP_134767.1; ZP_00379371.1; |
| | YP_134764.1; XP_742683.1; AAN29180.1; ZP_00602144.1; CAG35030.1; |
| | EAM31940.1; ZP_00556129.1; CAC49374.1; ZP_00050264.2; ZP_00379741.1; |
| | NP_705537.1; ZP_00471825.1; NP_104736.1; ZP_00600293.1; AAY87206.1; |
| | YP_266690.1; AAL76414.1; AAC31611.1; AAR38319.1; ZP_00658996.1; |
| | YP_266673.1; NP_105928.1; ZP_00630198.1; YP_266631.1; NP_254105.1; |
| | NP_107666.1; NP_104289.1; NP_102901.1; AAV96623.1; BAC74662.1; |
| | AAV95607.1; ZP_00620942.1; ZP_00645790.1; CAC41486.1; EAN05741.1; |
| | AAN65213.1; YP_269196.1; ZP_00620654.1; ZP_00660136.1; NP_885663.1; |
| | NP_436414.1; NP_881143.1; AAM75070.1; NP_572162.2; ZP_00379745.1; |
| | CAA39468.1; ZP_00264573.1; YP_262784.1; ZP_00380782.1; ZP_00278582.1; |
| | AAV94866.1; XP_414684.1; YP_237780.1; ZP_00602141.1; YP_165138.1; |
| | NP_790307.1; NP_620802.2; NP_534700.1; AAV95690.1; EAL32452.1; |
| | AAF21941.1; AAN65956.1; NP_083048.1; AAV94935.1; CAC46854.1; |
| | CAC41470.1; AAH24126.1; NP_037523.2; ZP_00565365.1; NP_102887.1; |
| | YP_134760.1; AAQ87217.1; AAH89599.1; ZP_00554816.1; AAK16482.1; |
| | NP_103085.1; ZP_00620147.1; NP_102906.1; ZP_00556188.1; XP_307967.2; |
| | ZP_00622120.1; CAC47100.1; NP_106776.1; CAH90377.1; ZP_00554918.1; |
| | CAD31640.1; XP_672544.1; AAT81177.1; AAK27867.2; AAD33412.1; |
| | NP_107653.1; CAI12276.1; BAD97122.1; ZP_00410737.1; AAD43585.1; |
| | NP_103793.1; XP_526883.1; XP_548398.1; CAB63337.2; YP_266661.1; |
| | ZP_00521798.1; EAA60688.1; AAL51865.1; AAV95026.1; EAL26357.1; |
| | ZP_00619907.1; CAE74368.1; NP_103190.1; AAV94915.1; AAG55663.1; |
| | XP_395831.2; AAK92969.1; NP_446116.1; AAF57796.1; AAN71380.1; |
| | ZP_00005411.1; CAE58942.1; EAA70894.1; AAV94873.1; ZP_00631887.1; |
| | ZP_00629673.1; BAB34711.1; ZP_00619923.1; ZP_00622863.1; EAL85410.1; |
| | AAL76413.1; AAR38318.1; CAD47921.1; AAH03456.1; NP_102854.1; |
| | AAH76859.1; AAL04443.1; AAH68953.1; YP_165137.1; XP_580581.1; |
| | ZP_00050273.2; ZP_00556400.1; ZP_00555517.1; AAV96627.1; AAV93943.1; |
| | AAH81271.1; AAK87410.1; ZP_00556086.1; AAV93879.1; AAH44792.1; |
| | XP_527208.1; ZP_00327983.1; ZP_00554213.1; NP_532318.1; CAI12274.1; |
| | AAH22388.1; XP_546052.1; XP_676622.1; AAV93533.1; YP_265671.1; |
| | AAV47350.1; AAV95190.1; CAD31286.1; ZP_00516133.1; ZP_00620892.1; |
| | AAH55193.1; AAY82706.1; ZP_00052785.2; AAR38102.1 |
| GcvL (LpdA) | P0A9P0; NP_706070.2; NP_752095.1; CAA24742.1; AAX64059.1; AAL19118.1; |
| protein | NP_804043.1; YP_149503.1; CAG76686.1; YP_069256.1; NP_930833.1; |
| | NP_935564.1; AAO10051.1; AAF95555.1; ZP_00585786.1; AAK02977.1; |
| | NP_798896.1; AAC46405.1; ZP_00122566.1; ZP_00132373.2; YP_131302.1; |
| | YP_205561.1; NP_716063.1; ZP_00637900.1; ZP_00633839.1; ZP_00582828.1; |
| | AAU37941.1; ZP_00134358.2; ZP_00157402.1; AAX88688.1; NP_439387.1; |
| | ZP_00154973.1; AAP96400.1; YP_154852.1; YP_271444.1; ZP_00464633.1; |
| | ZP_00451158.1; ZP_00212747.1; ZP_00500723.1; ZP_00486500.1; |
| | ZP_00463487.1; ZP_00467577.1; ZP_00423839.1; ZP_00423458.1; |
| | ZP_00283805.1; AAO90013.1; ZP_00595215.1; ZP_00170705.2; NP_879789.1; |
| | YP_123783.1; NP_889077.1; YP_126870.1; NP_240038.1; YP_095531.1; |
| | CAD15305.1; AAQ58205.1; NP_883762.1; NP_770362.1; YP_170418.1; |
| | CAA61894.1; AAV29309.1; CAB84783.1; AAF41719.1; ZP_00565931.1; |
| | CAA59171.1; CAA54878.1; AAW89295.1; CAA62435.1; ZP_00150164.2; 1BHY; |
| | 1OJT; CAA61895.1; YP_157096.1; CAA57206.1; AAM38502.1; NP_635936.1; |
| | YP_199361.1; NP_660554.1; NP_842161.1; ZP_00507350.1 NP_779995.1; |
| | YP_115390.1; ZP_00651360.1; NP_298158.1; AAR38073.1; EAO17659.1; |
| | ZP_00245305.1; AAR38213.1; AAR38090.1; NP_777818.1; BAC24467.1; |
| | NP_891227.1; NP_879460.1; NP_878457.1; CAD71978.1; YP_078853.1; |
| | AAK50273.1; AAK50266.1; AAF11916.1; NP_389344.1; ZP_00396676.1; |
| | EAO21015.1; CAA37631.1; 1EBD; YP_146914.1; BAB06371.1; YP_085309.1; |
| | AAP10890.1; YP_020826.1; YP_175913.1; Q04829; AAN03817.1; NP_692336.1; |
| | AAG17888.1; ZP_00474314.1; NP_764349.1; AAA99234.1; 1LPF; NP_250278.1; |
| | XP_475628.1; YP_253771.1; YP_143499.1; YP_257414.1; AAF34795.3; |
| | AAF79529.1; YP_040483.1; YP_005722.1; YP_074243.1; AAN23154.1; |
| | AAK50305.1; AAS20045.1; ZP_00540244.1; EAN07674.1; AAC26053.1; |
| | NP_815077.1; NP_908725.1; ZP_00307577.1; AAS47493.1; AAF34796.1; |
| | CAA11554.1; YP_013676.1; ZP_00317120.1; AAV48381.1; CAB84413.1; |
| | AAB30526.1; NP_969527.1; BAB44156.1; NP_464580.1; XP_635122.1; |
| | AAF41363.1; AAK50280.1; ZP_00397330.1; YP_265659.1; NP_470384.1; |
| | CAA44729.1; AAW89611.1; 1DXL; ZP_00401182.1 ZP_00418304.1; |
| | NP_792022.1; ZP_00625011.1; AAD53185.1; 3LAD; EAN08634.1; AAH18696.1; |
| | CAH93405.1; YP_235092.1; NP_967737.1; CAJ08862.1; NP_945538.1; |
| | NP_763632.1; BAE00452.1; BAD92940.1; NP_000099.1; IZMD; AAB01381.1; |
| | NP_999227.1; NP_767089.1; AAS47708.1; AAR21288.1; AAA35764.1; |
| | YP_034342.1; EAN90443.1; EAN96941.1; CAA61483.1; AAN69768.1; |
| | AAF12067.1; P31052; NP_105199.1; ZP_00263252.1; AAH62069.1; CAA72132.1; |
| | NP_031887.2; CAG58981.1; CAF26798.1; EAN80618.1; AAN15202.1; |
| | CAA72131.1; ZP_00269527.1; CAD72797.1; ZP_00554136.1; CAD61860.1; |
| | AAC53170.1; CAF05589.1; ZP_00622437.1; CAG81278.1; ZP_00284261.1; |
| | ZP_00497224.1; EAK93183.1; ZP_00492121.1; NP_533297.1; AAS53883.1; |
| | YP_160845.1; AAV93660.1; CAG31211.1; CAA49991.1; AAM93255.1; |
| | AAK11679.1; ZP_00427535.1; YP_258846.1; AAN30810.1; ZP_00449174.1; |
| | CAF92514.1; AAQ91233.1; AAH44432.1; XP_320877.2; AAH56016.1; |
| | YP_222565.1; CAC47627.1; AAA96487.1; ZP_00464142.1; NP_280867.1; |
| | YP_067405.1; CAB65609.1; AAL51327.1; AAK22329.1; YP_246823.1; |
| | NP_105334.1; XP_758608.1; CAH00655.1; NP_772974.1; AAB88282.1; |
| | ZP_00211386.1; EAN27796.1; AAN70931.1; EAL29693.1; ZP_00340462.1; |
| | ZP_00153792.2; ZP_00579524.1; AAZ17978.1; NP_266215.1; AAN33719.1; |
| | AAD30450.1; ZP_00383074.1; ZP_00597315.1; CAC47514.1; AAF49294.1; |
| | YP_223465.1; NP_220840.1; NP_360330.1; EAA26462.1; CAA39235.1; |
| | ZP_00578463.1; YP_047424.1; AAM36402.1; BAB03935.1; AAN69982.1; |
| | NP_116635.1; ZP_00654346.1; CAG85768.1; 1V59; NP_623271.1; AAA65618.1; |
| | ZP_00305550.1; XP_623438.1; ZP_00007570.1; ZP_00320049.1; AAN75183.1; |
| | ZP_00323583.1; YP_200681.1; 1LVL; ZP_00151187.2; AAP03132.1; |
| | CAD14973.1; ZP_00630163.1; ZP_00139957.1; NP_250940.1; NP_636857.1; |
| | CAB05249.2; ZP_00166998.2; AAN75720.1; CAA62982.1; ZP_00265019.1; |
| | ZP_00384289.1; AAV47687.1; ZP_00303079.1; NP_842316.1; XP_331183.1; |
| | AAV28779.1; AAN48422.1; ZP_00597992.1; AAN75618.1; AAV28746.1; |
| | ZP_00267415.1; ZP_00650982.1; AAQ58749.1; NP_298837.1; AAN75159.1; |
| | NP_778978.1; ZP_00575798.1; YP_002403.1; AAB97089.1; ZP_00511405.1; |
| | YP_005669.1; EAO21998.1; XP_613473.1; ZP_00245417.1; ZP_00210841.1; |
| | ZP_00561492.1; YP_259638.1; EAO16949.1; NP_785656.1; CAF23812.1; |
| | ZP_00055963.2; YP_143553.1; NP_953492.1; CAA63810.1; CAF22875.1; |
| | AAV89136.1; ZP_00536790.1; AAF39644.1; ZP_00621355.1; ZP_00486105.1; |
| | ZP_00020745.2; ZP_00589771.1; YP_180376.1; NP_220072.1; CAI27032.1; |
| | EAL87307.1; YP_112273.1; ZP_00376179.1; ZP_00498294.1; ZP_00492099.1; |
| | ZP_00463379.1; ZP_00217095.1; CAI27980.1; AAK23707.1; CAD60736.1; |
| | ZP_00268854.1; EAA77706.1; ZP_00629856.1; NP_879905.1; EAA51976.1; |
| | NP_885384.1; CAI29613.1; AAA91879.1; ZP_00376555.1; ZP_00141283.2; |
| | NP_253516.1; NP_300890.1; AAB40885.1; AAN03814.1; ZP_00644737.1; |
| | AAO36548.1; AAP98791.1; YP_079735.1; NP_388690.1; AAP05672.1; |
| | NP_966507.1; P95596; EAN04065.1; NP_532124.1; ZP_00507305.1; |
| | NP_948204.1; ZP_00557093.1; YP_220287.1; ZP_00642506.1; ZP_00591535.1; |
| | NP_102193.1; NP_771418.1; AAA19188.1; AAK72471.1; AAK72470.1; |
| | NP_345630.1; NP_756887.1; ZP_00404212.1; AAK72472.1; AAN50085.1; |
| | CAC46029.1; YP_001129.1; AAL64341.1; ZP_00526430.1; ZP_00308867.1; |
| | YP_053282.1; YP_036862.1; ZP_00210426.1; ZP_00625423.1; ZP_00601791.1; |
| | YP_045732.1; YP_016277.1; P54533; AAV95488.1; AAW71149.1; YP_021029.1; |
| | YP_153983.1; ZP_00240355.1; XP_395801.2; EAN08156.1; AAP94898.1; |
| | NP_326592.1; AAP11076.1; ZP_00239726.1; NP_980528.1; ZP_00620223.1; |
| | ZP_00512893.1; YP_019413.1; NP_148088.1; XP_678378.1; YP_180009.1; |
| | NP_735347.1; CAI26632.1; AAN30046.1; BAB04498.1; AAN57909.1; |
| | ZP_00373647.1; NP_692788.1; ZP_00053288.1; EAM72947.1; YP_078075.1; |
| | ZP_00006401.1; EAA16706.1; YP_221832.1; XP_742153.1; AAL52038.1; |
| | NP_966125.1; YP_247286.1; AAA74473.1; ZP_00589476.1; CAI38117.1; |
| | AAO78292.1; EAA26057.1; BAD11090.1; ZP_00545191.1; CAE73952.1; |
| | YP_060098.1; BAB05544.1; NP_802451.1; NP_360876.1; AAL97648.1; |
| | ZP_00154188.2; ZP_00331725.1; AAV62625.1; CAG35032.1; YP_084091.1; |
| | ZP_00366080.1; YP_139515.1; YP_121481.1; ZP_00340821.1; ZP_00531539.1; |
| | ZP_00630106.1; CAB06298.1; NP_979105.1;; ZP_00162168.1; AAP09729.1; |
| | YP_175948.1; NP_938751.1; ZP_00277446.1; BAB76444.1; YP_148232.1; |
| | AAK33923.1; AAO75416.1; AAT58044.1; YP_116014.1; BAD11095.1; |
| | P_00571989.1; YP_033412.1; AAT47753.1; NP_221155.1; ZP_00111840.1; |
| | ZP_00293744.1; CAF34426.1; YP_055934.1; EAM93810.1; NP_214976.1; |
| | NP_975267.1; NP_701672.1; NP_662186.1; AAG12404.1; NP_390286.1; |
| | AAB96096.1; BAB64316.1; YP_075992.1; ZP_00656450.1; ZP_00413985.1 |

In one example, the cell according to any aspect of the present invention may comprise at least a first genetic mutation that increases the expression relative to the wild type cell of an amino acid-N-acyl-transferase and a second genetic mutation that increases the expression of an acyl-CoA synthetase, and at least a third genetic mutation that decreases the expression relative to the wild type cell of at least one enzyme selected from the group consisting of an enzyme of the glycine cleavage system, glycine hydroxymethyltransferase (GlyA), threonine aldolase (LtaE), threonine dehydrogenase (Tdh), 2-Amino-3-Ketobutyrate CoA-Ligase (Kbl) and Allothreonine dehydrogenase (YdfG). This enzyme from the glycine cleavage system may be GcvT, GcvP, GcvL or GcvH. In particular, the expression of GcvT and GcvP, GcvT and GcvH or GcvP and GcvH is decreased. In one example, the expression of GcvT, GcvP and GcvH may be decreased. More in particular, the sequence of GcvT, GcvP and Gcvh may be SEQ ID NO:58, SEQ ID NO:60 and SEQ ID NO:59 respectively. The sequence of GlyA may be SEQ ID NO:61, the sequence of LtaE may be SEQ ID NO:62, the sequence of Tdh may be SEQ ID NO:81, the sequence of Kbl may be SEQ ID NO:80 and the sequence of YdfG may be SEQ ID NO:82.

In another example, the cell according to any aspect of the present invention may comprise at least a first genetic mutation that increases the expression relative to the wild type cell of an amino acid-N-acyl-transferase and a second genetic mutation that increases the expression of an acyl-CoA synthetase, and at least a third genetic mutation that decreases the expression relative to the wild type cell of GcvT, GcvP, GcvH, GlyA and LtaE; or GcvT, GcvP, GcvH, and GlyA; or GcvT, GcvP, GcvH, and LtaE; or GcvT and GcvH and GlyA and/or LtaE; or GcvP and GcvH and GlyA and/or LtaE; or GcvT and GcvP and GlyA and/or LtaE; or GcvT, GcvP, GcvH, GlyA and Tdh; or GcvT, GcvP, GcvH, and Tdh; or GcvT and GcvH and GlyA and/or Tdh; or GcvP and GcvH and GlyA and/or Tdh; or GcvT and GcvP and GlyA and/or Tdh; or GcvT, GcvP, GcvH, GlyA and Kbl; or GcvT, GcvP, GcvH, and Kbl; or GcvT and GcvH and GlyA and/or Kbl; or GcvP and GcvH and GlyA and/or Kbl; or GcvT and GcvP and GlyA and/or Kbl, or GcvT, GcvP, GcvH, GlyA and YdfG; or GcvT, GcvP, GcvH, and YdfG; or GcvT and GcvH and GlyA and/or YdfG; or GcvP and GcvH and GlyA and/or YdfG; or GcvT and GcvP and GlyA and/or YdfG;

In one example, the cell according to any aspect of the present invention may comprise at least a first genetic mutation that increases the expression relative to the wild type cell of an amino acid-N-acyl-transferase and a second genetic mutation that increases the expression of an acyl-CoA synthetase, and at least a third genetic mutation that decreases the expression relative to the wild type cell of GcvT, GcvP, GcvH and at least one enzyme selected from the group consisting of LtaE, Tdh, Kbl and YdfG. In another example, the cell according to any aspect of the present invention may comprise at least a first genetic mutation that increases the expression relative to the wild type cell of an amino acid-N-acyl-transferase and a second genetic mutation that increases the expression of an acyl-CoA synthetase, and at least a third genetic mutation that decreases the expression relative to the wild type cell of GlyA and at least one enzyme selected from the group consisting of LtaE, Tdh, Kbl and YdfG. In particular, the cell according to any aspect of the present invention may comprise at least a first genetic mutation that increases the expression relative to the wild type cell of an amino acid-N-acyl-transferase and a second genetic mutation that increases the expression of an acyl-CoA synthetase, and at least a third genetic mutation that decreases the expression relative to the wild type cell of GcvT, GcvP, GcvH, GlyA and at least one enzyme selected from the group consisting of LtaE, Tdh, Kbl and YdfG. For example, the cell according to any aspect of the present invention, may comprise
at least a first genetic mutation that increases the expression relative to the wild type cell of an amino acid-N-acyl-transferase,
at least a second genetic mutation that increases the expression relative to the wild type cell of an acyl-CoA synthetase, and
at least a third genetic mutation that decreases the expression relative to the wild type cell of at least GcvT, GcvP, GcvH and LtaE; or
at least a third genetic mutation that decreases the expression relative to the wild type cell of at least GcvT, GcvP, GcvH and Tdh; or
at least a third genetic mutation that decreases the expression relative to the wild type cell of at least GcvT, GcvP, GcvH and Kbl; or
at least a third genetic mutation that decreases the expression relative to the wild type cell of at least GcvT, GcvP, GcvH and YdfG; or
at least a third genetic mutation that decreases the expression relative to the wild type cell of at least GlyA and LtaE; or
at least a third genetic mutation that decreases the expression relative to the wild type cell of at least GlyA and Tdh; or
at least a third genetic mutation that decreases the expression relative to the wild type cell of at least GlyA and Kbl; or
at least a third genetic mutation that decreases the expression relative to the wild type cell of at least GlyA and YdfG; or
at least a third genetic mutation that decreases the expression relative to the wild type cell of at least GcvT, GcvP, GcvH and GlyA and LtaE; or
at least a third genetic mutation that decreases the expression relative to the wild type cell of at least GcvT, GcvP, GcvH and GlyA and Tdh; or
at least a third genetic mutation that decreases the expression relative to the wild type cell of at least GcvT, GcvP, GcvH and GlyA and Kbl; or
at least a third genetic mutation that decreases the expression relative to the wild type cell of at least GcvT, GcvP, GcvH and GlyA and YdfG; or
at least a third genetic mutation that decreases the expression relative to the wild type cell of at least GcvT, GcvP, GcvH, GlyA, LtaE and Kbl.

The expression of GcvL may be maintained or decreased according to any aspect of the present invention. The phrase “decreased activity of an enzyme” as used herein is to be understood as decreased intracellular activity. Basically, a decrease in enzymatic activity can be achieved by decreasing the copy number of the gene sequence or gene sequences that code for the enzyme, using a weak promoter or employing a gene or allele that silence the respective enzyme to decrease activity and optionally by combining these measures. Genetically modified cells used according to any aspect of the present invention are for example produced by transformation, transduction, conjugation or a combination of these methods with a vector that contains a silencer that binds to this gene or parts thereof and a vector that makes switches off the gene possible.

The cell according to any aspect of the present invention may comprise a further genetic mutation that results in the cell having a reduced fatty acid degradation capacity relative to the wild type cell. In particular, the reduced fatty acid degradation capacity may be a result of a decrease in expression relative to the wild type cell of at least one enzyme selected from the group consisting of acyl-CoA dehydrogenase, 2,4-dienoyl-CoA reductase, enoyl-CoA hydratase and 3-ketoacyl-CoA thiolase

In particular, the term “having a reduced fatty acid degradation capacity”, as used herein, means that the respective cell degrades fatty acids, especially those taken up from the environment, at a lower rate than a comparable cell or wild type cell having normal fatty acid degradation capacity would under identical conditions. More in particular, the fatty acid degradation of such a cell is lower on account of deletion, inhibition or inactivation of at least one gene encoding an enzyme involved in the n-oxidation pathway. In one example, at least one enzyme involved in the β-oxidation pathway has lost 5, 10, 20, 40, 50, 75, 90 or 99% activity relative to the activity of the same enzyme under comparable conditions in the respective wild type microorganism. The person skilled in the art is familiar with various techniques that may be used to delete a gene encoding an enzyme or reduce the activity of such an enzyme in a cell, for example by exposition of cells to radioactivity followed by accumulation or screening of the resulting mutants, site-directed introduction of point mutations or knock out of a chromosomally integrated gene encoding for an active enzyme, as described in Sambrook/Fritsch/Maniatis (1989). In addition, the transcriptional repressor FadR may be over expressed to the effect that expression of enzymes involved in the β-oxidation pathway is repressed (Fujita, Y., 2007).

The term “deletion of a gene”, as used herein, means that the nucleic acid sequence encoding the gene may be modified such that the expression of active polypeptide encoded by the gene is reduced. For example, the gene may be deleted by removing in-frame a part of the sequence comprising the sequence encoding for the catalytic active centre of the polypeptide. Alternatively, the ribosome binding site may be altered such that the ribosomes no longer translate the corresponding RNA. The person skilled in the art is able to routinely measure the activity of enzymes expressed by living cells using standard assays as described in enzymology text books, for example Cornish-Bowden (1995).

Degradation of fatty acids may be accomplished by a sequence of enzymatically catalysed reactions. First of all, fatty acids are taken up and translocated across the cell membrane via a transport/acyl-activation mechanism involving at least one outer membrane protein and one inner membrane-associated protein which has fatty acid-CoA ligase activity, referred to in the case of E. coli as FadL and FadD/FadK, respectively. Inside the cell, the fatty acid to be degraded is subjected to enzymes catalysing other reactions of the β-oxidation pathway. The first intracellular step involves the conversion of acyl-CoA to enoyl-CoA through acyl-CoA dehydrogenase, the latter referred to as FadE in the case of E. coli. The activity of an acyl-CoA dehydrogenase may be assayed using any method known in the art. For example by monitoring the concentration of NADH spectrophotometrically at 340 nm in 100 mM MOPS, pH 7.4, 0.2 mM Enoyl-CoA, 0.4 mM NAD+. The resulting enoyl-CoA is converted to 3-ketoacyl-CoA via 3-hydroxylacyl-CoA through hydration and oxidation, catalysed by enoyl-CoA hydratase/3-hydroxyacyl-CoA dehydrogenase, referred to as FadB and FadJ in E. coli. Enoyl-CoA hydratase/3-hydroxyacyl-CoA dehydrogenase activity, more specifically formation of the product NADH may be assayed spectrophotometrically as described in the state of the art, for example as outlined for FadE. Finally, 3-ketoacyl-CoA thiolase, FadA and Fadl in E. coli, catalyses the cleavage of 3-ketoacyl-CoA, to give acetyl-CoA and the input acyl-CoA shortened by two carbon atoms. The activity of ketoacyl-CoA thiolase may be assayed as described in the state of the art, for example in Antonenkov, V., 1997.

In particular, the term “a cell having a reduced fatty acid degradation capacity”, as used herein, refers to a cell having a reduced capability of taking up and/or degrading fatty acids, in particular those having at least eight carbon chains. The fatty acid degradation capacity of a cell may be reduced in various ways. In a one example, the cell has, compared to its wild type, a reduced activity of an enzyme involved in the β-oxidation pathway. In one example, the term “enzyme involved in the β-oxidation pathway”, as used herein, refers to an enzyme that interacts directly with a fatty acid or a derivative thereof formed as part of the degradation of said fatty acid via the β-oxidation pathway the sequence of reactions effecting the conversion of a fatty acid to acetyl-CoA and the CoA ester of the shortened fatty acid, preferably by recognizing the fatty acid or derivative thereof as a substrate, and converts it to a metabolite formed as a part of the β-oxidation pathway. For example, the acyl-CoA dehydrogenase is an enzyme involved in the β-oxidation pathway as it interacts with fatty acid-CoA and converts fatty acid-CoA ester to enoyl-CoA, which is a metabolite formed as part of the β-oxidation. In one example, the term “enzyme involved in the β-oxidation pathway”, as used herein, comprises any polypeptide from the group comprising acyl-CoA dehydrogenase, enoyl-CoA hydratase, 3-hydroxyacyl-CoA dehydrogenase and 3-keto-acyl-CoA thiolase. Subsequently, the acyl-CoA synthetase may catalyse the conversion a fatty acid to the CoA ester of a fatty acid, i.e. a molecule, wherein the functional group —OH of the carboxy group is replaced with —S—CoA, preferably for introducing said fatty acid into the β-oxidation pathway. For example, the polypeptides FadD and FadK in E. coli (Accession number: BAA15609.1) are acyl-CoA dehydrogenases. In one example, the term “acyl-CoA dehydrogenase”, as used herein, is a polypeptide capable of catalysing the conversion of an acyl-CoA to enoyl-CoA, preferably as part of the β-oxidation pathway. For example, the polypeptide FadE in E. coli (Accession number: BAA77891.2) is an acyl-CoA dehydrogenase. In particular, the term “2,4-dienoyl-CoA reductase”, as used herein, is a polypeptide capable of catalysing the conversion of the 2,4-dienoyl CoA from an unsaturated fatty acid into enoyl-CoA, more in particular as part of the β-oxidation pathway. For example, the polypeptide FadH in E. coli is a 2,4-dienoyl-CoA reductase. In one example, the term “enoyl-CoA hydratase”, as used herein, also referred to as 3-hydroxyacyl-CoA dehydrogenase, refers to a polypeptide capable of catalysing the conversion of enoyl-CoA to 3-ketoacyl-CoA through hydration and oxidation, preferably as part of the β-oxidation pathway. For example, the polypeptides FadB and FadJ in E. coli (Accession number: BAE77457.1) are enoyl-CoA hydratases. In particular, the term “ketoacyl-CoA thiolase”, as used herein, refers to a polypeptide capable of catalysing the conversion of cleaving 3-ketoacyl-CoA, resulting in an acyl-CoA shortened by two carbon atoms and acetyl-CoA, as the final step of the β-oxidation pathway. For example, the polypeptides FadA and Fadl in E. coli (Accession number: AP009048.1) are ketoacyl-CoA thiolases.

In one example, the cells according to any aspect of the present invention may be genetically modified to result in an increased activity of at least one amino acid N-acyl transferase in combination with increased activity of at least one acyl-CoA synthetase in combination with an increased activity of at least one transporter protein of the FadL and/or the AlkL. In particular, the cell according to any aspect of the present invention may be genetically modified to overexpress glycine N-acyl transferase, acyl-CoA/ACP synthetase, a transporter protein of the FadL and the AlkL compared to the wild type cell. These cells may be capable of producing acyl glycinates. In another example, the cell according to any aspect of the present invention may be genetically modified compared to the wild type cell to:
increase the expression of amino acid N-acyl transferase, acyl-CoA synthetase; and
reduce activity of at least one enzyme selected from the group consisting of acyl-CoA dehydrogenase FadE, multifunctional 3-hydroxybutyryl-CoA epimerase, Δ3-cis-Δ2-trans-enoyl-CoA isomerase, enoyl-CoA hydratase, 3-hydroxyacyl-CoA dehydrogenase FadB, 3-ketoacyl-CoA thiolase and electron-transfer flavoprotein.

In yet another example, the cells according to any aspect of the present invention may be genetically modified compared to the wild type cell to result in:
increase the expression of amino acid N-acyl transferase, acyl-CoA synthetase and at least one transporter protein of the FadL and/or the AlkL; and
reduce activity of at least one enzyme selected from the group consisting of acyl-CoA dehydrogenase FadE, multifunctional 3-hydroxybutyryl-CoA epimerase, Δ3-cis-Δ2-trans-enoyl-CoA isomerase, enoyl-CoA hydratase, 3-hydroxyacyl-CoA dehydrogenase FadB, 3-ketoacyl-CoA thiolase and electron-transfer flavoprotein.

In particular, the acyl-CoA dehydrogenase FadE (EC 1.3.8.7, EC 1.3.8.8 or EC 1.3.8.9) may catalyse the reaction acyl-CoA+electron-transfer flavoprotein=trans-2,3-dehydroacyl-CoA+reduced electron-transfer flavoprotein; the multifunctional 3-hydroxybutyryl-CoA epimerase (EC 5.1.2.3), Δ3-cis-Δ2-trans-enoyl-CoA isomerase (EC 5.3.3.8), enoyl-CoA hydratase (EC 4.2.1.17) and 3-hydroxyacyl-CoA dehydrogenase (EC 1.1.1.35) FadB, may catalyse the reactions (S)-3-hydroxybutanoyl-CoA→(R)-3-hydroxybutanoyl-CoA, (3Z)-3-enoyl-CoA→(2E)-2-enoyl-CoA, (3S)-3-hydroxyacyl-CoA 4 trans-2-enoyl-CoA+H2O, (S)-3-hydroxyacyl-CoA+NAD+=3-oxoacyl-CoA+NADH+H+; 3-ketoacyl-CoA thiolase (EC 2.3.1.16), may catalyse the reaction acyl-CoA+acetyl-CoA 4 CoA+3-oxoacyl-CoA; and electron-transfer flavoprotein (EC 1.5.5.1), may catalyse the following reaction: reduced electron-transferring flavoprotein+ubiquinone 4 electron-transferring flavoprotein+ubiquinol.

More in particular, the cell according to any aspect of the present invention may be genetically modified compared to the wild type cell to increase the expression of glycine N-acyl transferase, acyl-CoA/ACP synthetase, a transporter protein of the FadL and the AlkL; and reduce activity of acyl-CoA dehydrogenase FadE, multifunctional 3-hydroxybutyryl-CoA epimerase, Δ3-cis-Δ2-trans-enoyl-CoA isomerase, enoyl-CoA hydratase, 3-hydroxyacyl-CoA dehydrogenase FadB, 3-ketoacyl-CoA thiolase and electron-transfer flavoprotein.

In one example, the cell according to any aspect of the present invention may be genetically modified compared to the wild type cell to:
increase the expression of amino acid N-acyl transferase, acyl-CoA synthetase; and
reduce activity of at least one enzyme selected from the group consisting of glycine cleavage system H protein, glycine cleavage system P protein, glycine cleavage system L protein, glycine cleavage system T protein, threonine aldolase, glycine hydroxylmethyltransferase, threonine dehydrogenase, 2-Amino-3-Ketobutyrate CoA-Ligase and Allothreonine dehydrogenase.

In another example, the cell according to any aspect of the present invention may be genetically modified compared to the wild type cell to:
increase the expression of amino acid N-acyl transferase, acyl-CoA synthetase and at least one transporter protein of the FadL and/or the AlkL; and
reduce activity of at least one enzyme selected from the group consisting of acyl-CoA dehydrogenase FadE, multifunctional 3-hydroxybutyryl-CoA epimerase, Δ3-cis-Δ2-trans-enoyl-CoA isomerase, enoyl-CoA hydratase, 3-hydroxyacyl-CoA dehydrogenase FadB, 3-ketoacyl-CoA thiolase, and electron-transfer flavoprotein.

In yet another example, the cell according to any aspect of the present invention may be genetically modified compared to the wild type cell to:
increase the expression of amino acid N-acyl transferase, acyl-CoA synthetase and at least one transporter protein of the FadL and/or the AlkL;
reduce activity of at least one enzyme selected from the group consisting of acyl-CoA dehydrogenase FadE, multifunctional 3-hydroxybutyryl-CoA epimerase, Δ3-cis-Δ2-trans-enoyl-CoA isomerase, enoyl-CoA hydratase, 3-hydroxyacyl-CoA dehydrogenase FadB, 3-ketoacyl-CoA thiolase, and electron-transfer flavoprotein; and
reduce activity of at least one enzyme selected from the group consisting of glycine cleavage system H protein, glycine cleavage system P protein, glycine cleavage system L protein, glycine cleavage system T protein, threonine aldolase, and glycine hydroxylmethyltransferase, threonine dehydrogenase, 2-Amino-3-Ketobutyrate CoA-Ligase and Allothreonine dehydrogenase.

In particular, the cell according to any aspect of the present invention may be genetically modified compared to the wild type cell to increase the expression of glycine N-acyl transferase, acyl-CoA/ACP synthetase, a transporter protein of the FadL and the AlkL; reduce activity of acyl-CoA dehydrogenase FadE, multifunctional 3-hydroxybutyryl-CoA epimerase, Δ3-cis-Δ2-trans-enoyl-CoA isomerase, enoyl-CoA hydratase, 3-hydroxyacyl-CoA dehydrogenase FadB, 3-ketoacyl-CoA thiolase, electron-transfer flavoprotein; and reduce activity of glycine cleavage system H protein, glycine cleavage system P protein, glycine cleavage system L protein, glycine cleavage system T protein, threonine aldolase, glycine hydroxylmethyltransferase, threonine dehydrogenase, 2-Amino-3-Ketobutyrate CoA-Ligase and Allothreonine dehydrogenase.

More in particular, the glycine cleavage system H protein, carrying lipoic acid and interacts with the glycine cleavage system proteins P, L and T; the glycine cleavage system P protein (EC 1.4.4.2), catalyses the reaction glycine+[glycine-cleavage complex H protein]-N(6)-lipoyl-L-lysine→[glycine-cleavage complex H protein]-S-aminomethyl-N(6)-dihydrolipoyl-L-lysine+CO2; glycine cleavage system L protein (EC 1.8.1.4), catalyses the reaction protein N6-(dihydrolipoyl)lysine+NAD+→protein N6-(lipoyl)lysine+NADH+H+; glycine cleavage system T protein (EC 2.1.2.10), catalyses the reaction [protein]-S8-aminomethyldihydrolipoyllysine+tetrahydrofolate→[protein]-dihydrolipoyllysine+5,10-methylenetetrahydrofolate+NH₃; threonine aldolase (EC 4.2.1.48), catalyses the reaction L-threonine→glycine+acetaldehyde; serine hydroxylmethyltransferase (EC 2.1.2.1), catalyses the reaction 5,10-methylenetetrahydrofolate+glycine+H₂O+tetrahydrofolate+L-serine.

The fatty acids that are to be converted to acyl amino acids may be produced by the cell according to any aspect of the present invention. In one example, the very cell that produces the acyl amino acids may be capable of producing the fatty acids from which the acyl amino acids are produced. In particular, the cells may be genetically modified to be able to produce fatty acids. In one example, the genetic modification may be to decrease a specific enzymatic activity and this may be done by a gene disruption or a genetic modification. The genetic modification may also increase a specific enzymatic activity. In particular, the genetic modification may increase microbial synthesis of a selected fatty acid or fatty acid derived chemical product above a rate of a control or wild type cell. This control or wild type cell may lack this genetic modification to produce a selected chemical product. Cells which may be able to produce fatty acids and/or proteinogenic amino acids are at least described in US20140051136. The fatty acid may be an unsaturated fatty acid and may be selected from the group consisting of myristoleic acid, lauroleic acid, palmitoleic acid and cis-vaccenic acid. In another example, the fatty acid may be a saturated fatty acid and may be selected from the group consisting of laurate, myristate and palmitate. The fatty acid according to any aspect of the present invention may be provided in the form of an organic phase comprising a liquid organic solvent and the fatty acid, wherein the organic solvent may be an ester of the fatty acid.

In one example, the cell according to any aspect of the present invention may be genetically modified to increase the expression of amino acid N-acyl transferase, acyl-CoA synthetase, and comprise a genetic modification in the cell capable of producing at least one fatty acid from at least one carbohydrate. A list of non-limiting genetic modification to enzymes or enzymatic activities is provided below in Table 2. The cells according to any aspect of the present invention may comprise a combination of genetic modification that produce fatty acids and convert the fatty acids to N-acyl amino acids. In particular, the cell according to any aspect of the present invention may be genetically modified to increase the expression of amino acid N-acyl transferase, acyl-CoA synthetase and comprise any of the genetic modifications listed in Table 2. More in particular, the cell may be genetically modified to increase the expression of N-acyl transferase, acyl-CoA synthetase, a transporter protein of the FadL and the AlkL and comprise any of the genetic modifications listed in Table 2. Even more in particular, the cell may be genetically modified to increase the expression of N-acyl transferase, acyl-CoA synthetase, a transporter protein of the FadL and the AlkL, reduce activity of acyl-CoA dehydrogenase FadE, multifunctional 3-hydroxybutyryl-CoA epimerase, Δ3-cis-Δ2-trans-enoyl-CoA isomerase, enoyl-CoA hydratase, 3-hydroxyacyl-CoA dehydrogenase FadB, 3-ketoacyl-CoA thiolase, electron-transfer flavoprotein; reduce activity of glycine cleavage system H protein, glycine cleavage system P protein, glycine cleavage system L protein, glycine cleavage system T protein, threonine aldolase, glycine hydroxylmethyltransferase, threonine dehydrogenase, 2-Amino-3-Ketobutyrate CoA-Ligase and allothreonine dehydrogenase, and comprise any of the genetic modifications listed in Table 2.

In one example, the AlkL may comprise an amino acid sequence comprising 50, 60, 65, 70, 80, 85, 90, 92, 94, 95, 96, 97, 98 or 99% sequence identity in relation to SEQ ID NO: 78 and/or the FadL may comprise an amino acid sequence comprising 50, 60, 65, 70, 80, 85, 90, 92, 94, 95, 96, 97, 98 or 99% sequence identity in relation to SEQ ID NO: 79.

In one example, the cell according to any aspect of the present invention may be genetically modified to increase the expression of amino acid N-acyl transferase, acyl-CoA/ACP synthetase and decrease the expression of at least one enzyme selected from the group consisting of β-ketoacyl-ACP synthase I, 3-oxoacyl-ACP-synthase I, Malonyl-CoA-ACP transacylase, enoyl ACP reductase, and β-ketoacyl-ACP synthase III. In particular, the genetic modification in the cell according to any aspect of the present invention may comprise an increase in the expression compared to the wild type cell of amino acid N-acyl transferase, acyl-CoA/ACP synthetase and a decrease in the expression of β-ketoacyl-ACP synthase I, 3-oxoacyl-ACP-synthase I, Malonyl-CoA-ACP transacylase, enoyl ACP reductase, and β-ketoacyl-ACP synthase III.

Accordingly, the cells and methods of the present invention may comprise providing a genetically modified microorganism that comprises both a production pathway to a fatty acid or fatty acid derived product, and a modified polynucleotide that encodes an enzyme of the malonyl-ACP dependent fatty acid synthase system that exhibits reduced activity, so that utilisation of malonyl-CoA shifts toward the production pathway compared with a comparable (control) microorganism lacking such modifications. The methods involve producing the chemical product using a population of such genetically modified microorganism in a vessel, provided with a nutrient media. Other genetic modifications described herein, to other enzymes, such as acetyl-CoA carboxylase and/or NADPH-dependent transhydrogenase, may be present in some such examples. Providing additional copies of polynucleotides that encode polypeptides exhibiting these enzymatic activities is shown to increase a fatty acid or fatty acid derived product production. Other ways to increase these respective enzymatic activities is known in the art and may be applied to various examples of the present invention.

Also, without being limiting, a first step in some multi-phase methods of making a fatty acid may be exemplified by providing into a vessel, such as a culture or bioreactor vessel, a nutrient media, such as a minimal media as known to those skilled in the art, and an inoculum of a genetically modified microorganism so as to provide a population of such microorganism, such as a bacterium, and more particularly a member of the family Enterobacteriaceae, such as E. coli, where the genetically modified microorganism comprises a metabolic pathway that converts malonyl-CoA to a fatty acid. This inoculum is cultured in the vessel so that the cell density increases to a cell density suitable for reaching a production level of a fatty acid or fatty acid derived product that meets overall productivity metrics taking into consideration the next step of the method. In various alternative embodiments, a population of these genetically modified microorganisms may be cultured to a first cell density in a first, preparatory vessel, and then transferred to the noted vessel so as to provide the selected cell density. Numerous multi-vessel culturing strategies are known to those skilled in the art. Any such methods provide the selected cell density according to any aspect of the present invention.

Without being limiting, a subsequent step may be exemplified by two approaches, which also may be practiced in combination in various embodiments. A first approach provides a genetic modification to the genetically modified microorganism such that its enoyl-ACP reductase enzymatic activity may be controlled. As one example, a genetic modification may be made to substitute a temperature-sensitive mutant enoyl-ACP reductase (e.g., fabl^{TS} in E. coli) for the native enoyl-ACP reductase. The former may exhibit reduced enzymatic activity at temperatures above 30° C. but normal enzymatic activity at 30° C., so that elevating the culture temperature to, for example to 34° C., 35° C., 36° C., 37° C. or even 42° C., reduces enzymatic activity of enoyl-ACP reductase. In such case, more malonyl-CoA is converted to a fatty acid or fatty acid derived product or another chemical product than at 30° C., where conversion of malonyl-CoA to fatty acids is not impeded by a less effective enoyl-ACP reductase.

Other genetic modifications that may be useful in the production of fatty acids and/or amino acids may be included in the cell according to any aspect of the present invention. For example, the ability to utilize sucrose may be provided, and this would expand the range of feed stocks that can be utilized to produce a fatty acid or fatty acid derived product or other chemical products. Common laboratory and industrial strains of E. coli, such as the strains described herein, are not capable of utilizing sucrose as the sole carbon source. Since sucrose, and sucrose-containing feed stocks such as molasses, are abundant and often used as feed stocks for the production by microbial fermentation, adding appropriate genetic modifications to permit uptake and use of sucrose may be practiced in strains having other features as provided herein. Various sucrose uptake and metabolism systems are known in the art (for example, U.S. Pat. No. 6,960,455).

Also, genetic modifications may be provided to add functionality for breakdown of more complex carbon sources, such as cellulosic biomass or products thereof, for uptake, and/or for utilization of such carbon sources. For example, numerous cellulases and cellulase-based cellulose degradation systems have been studied and characterized (Beguin, P 1994 and Ohima, K. et al., 1997).

In some examples, genetic modifications increase the pool and availability of the cofactor NADPH, and/or, consequently, the NADPH/NADP⁺ ratio may also be provided. For example, in E. coli, this may be done by increasing activity, such as by genetic modification, of one or more of the following genes: pgi (in a mutated form), pntAB, overexpressed, gapA:gapN substitution/replacement, and disrupting or modifying a soluble transhydrogenase such as sthA, and/or genetic modifications of one or more of zwf, gnd, and edd.

Any such genetic modifications may be provided to species not having such functionality, or having a less than desired level of such functionality. More generally, and depending on the particular metabolic pathways of a microorganism selected for genetic modification, any subgroup of genetic modifications may be made to decrease cellular production of fermentation product(s) selected from the group consisting of acetate, acetoin, acetone, acrylic, malate, fatty acid ethyl esters, isoprenoids, glycerol, ethylene glycol, ethylene, propylene, butylene, isobutylene, ethyl acetate, vinyl acetate, other acetates, 1,4-butanediol, 2,3-butanediol, butanol, isobutanol, sec-butanol, butyrate, isobutyrate, 2-OH-isobutryate, 3-OH-butyrate, ethanol, isopropanol, 0-lactate, L-lactate, pyruvate, itaconate, levulinate, glucarate, glutarate, caprolactam, adipic acid, propanol, isopropanol, fusel alcohols, and 1,2-propanediol, 1,3-propanediol, formate, fumaric acid, propionic acid, succinic acid, valeric acid, and maleic acid. Gene deletions may be made as disclosed generally herein, and other approaches may also be used to achieve a desired decreased cellular production of selected fermentation products.

In particular, the cell according to any aspect of the present invention may comprise at least:
a first genetic mutation that increases the expression relative to the wild type cell of an amino acid-N-acyl-transferase that may be the human amino acid-N-acyl-transferase;
at least a second genetic mutation that increases the expression relative to the wild type cell of an acyl-CoA synthetase, and
at least a third genetic mutation that decreases the expression relative to the wild type cell of at least one enzyme selected from the group consisting of an enzyme of the glycine cleavage system, glycine hydroxymethyltransferase (GlyA), threonine aldolase (LtaE), threonine dehydrogenase (Tdh), 2-Amino-3-Ketobutyrate CoA-Ligase (Kbl), and allothreonine dehydrogenase (YdfG).

In particular, the amino acid-N-acyl-transferase has 50, 60, 70, 75, 80, 85, 90, 92, 94, 95, 97, 98, 99% sequence identity to SEQ ID NO:63 or SEQ ID NO:64. More in particular, the amino acid-N-acyl-transferase has 85% sequence identity to SEQ ID NO:63 or SEQ ID NO:64.

TABLE 2
Examples of genetic modifications in cells of microorganisms for production of fatty acids
Genetic Modifications

| | E.C. CLASSIFI- | GENE NAME | |
|---|---|---|---|
| ENZYME FUNCTION | CATION No. | IN E. COLI | COMMENTS |
| Glucose transporter | N/A | galP | Increase function |
| Pyruvate dehydrogenase E1p | 1.2.4.1 | aceE | Increase function |
| lipoate acetyltransferase/ | 2.3.1.12 | aceF | Increase function |
| dihydrolipoamide acetyltransferase | | | |
| Pyruvate dehydrogenase | 1.8.1.4 | lpd | Increase function or |
| E3 (lipoamide dehydrogenase) | | | alter such as by |
| | | | mutation to increase |
| | | | resistance to NADH |
| | | | inhibition, |
| Lactate dehydrogenase | 1.1.1.28 | ldhA | Decrease function, |
| | | | including by mutation |
| Pyruvate formate lyase | 2.3.1.— | pflB | Decrease function, |
| (B “inactive”) | | | including by mutation |
| Pyruvate oxidase | 1.2.2.2 | poxB | Decrease function, |
| | | | including by mutation |
| Phosphate acetyltransferase | 2.3.1.8 | Pta | Decrease function, |
| | | | including by mutation |
| acetate kinase | 2.7.2.15 2.7.2.1 | ackA | Decrease function, |
| | | | including by mutation |
| methylglyoxal synthase | 4.2.3.3 | mgsA | Decrease function, |
| | | | including by mutation |
| Heat stable, histidyl | N/A | ptsH | Decrease function, |
| phosphorylatable protein (of PTS) | | (HPr) | including by mutation |
| Phosphoryl transfer protein | N/A | ptsI | Decrease function, |
| (of PTS) | | | including by mutation |
| Polypeptide chain (of PTS) | N/A | Crr | Decrease function, |
| | | | including by mutation |
| 3-oxoacyl-ACP synthase I | 2.3.1.179 | fabF | Decrease function, |
| 3-oxoacyl-ACP synthase II | 2.3.1.41 | | including by mutation |
| monomer | | | |
| β-ketoacyl-ACP synthase I, | 2.3.1.41 | fabB | Decrease function, |
| 3-oxoacyl-ACP-synthase I | 2.3.1.— | | including by mutation |
| Malonyl-CoA-ACP | 2.3.1.39 | fabD | Decrease function, |
| transacylase | | | including by mutation |
| enoyl acyl carrier protein | 1.3.1.9, | fabI | Decrease function, |
| reductase | 1.3.1.10 | | including by mutation |
| β-ketoacyl-acyl carrier | 2.3.1.180 | fabH | Decrease function, |
| protein synthase III | | | including by mutation |
| Carboxyl transferase | 6.4.1.2 | accA | Increase function |
| subunit α subunit | | | |
| Biotin carboxyl carrier | 6.4.1.2 | accB | Increase function |
| protein | | | |
| Biotin carboxylase subunit | 6.3.4.14 | accC | Increase function |
| Carboxyl transferase | 6.4.1.2 | accD | Increase function |
| subunit β subunit | | | |
| long chain fatty acyl | 3.1.2.2, | tesA | Increase function as |
| thioesterase I | 3.1.1.5 | | well as alter by |
| | | | mutation to express in |
| | | | cytoplasm or deletion |
| acyl-CoA synthase | 2.3.1.86 | fadD | Decrease via deletion |
| | | | or mutation |
| acetate CoA-transferase | 2.8.3.8 | atoD | Decrease via deletion |
| | | | or mutation |
| acetate CoA-transferase | 2.8.3.8 | atoA | Decrease via deletion |
| | | | or mutation |
| Transporter | N/A | atoE | Decrease via deletion |
| | | | or mutation |
| acetyl-CoA acetyltransferase | 2.3.1.9 | atoB | Decrease via deletion |
| | | | or mutation |
| pantothenate kinase | 2.7.1.33 | coaA | Increase via expression |
| | | | or feedback resistant |
| | | | mutation |
| lactose repressor | N/A | lacI | Decrease via deletion |
| | | | or mutation |
| γ-glutamyl-γ- | 1.2.1.— | puuC | Decrease via deletion |
| aminobutyraldehyde | | | or mutation |
| dehydrogenase | | | |
| malate synthase A | 2.3.3.9 | aceB | Decrease via deletion |
| | | | or mutation |
| isocitrate lyase | 4.1.3.1 | aceA | Decrease via deletion |
| | | | or mutation |
| isocitrate dehydrogenase | 3.1.3.— | aceK | Decrease via deletion |
| phosphatase/isocitrate | 2.7.11.5. | | or mutation |
| dehydrogenase kinase | | | |
| pyruvate formate-lyase | 1.2.1.10 1.1.1.1 | adhE | Decrease via deletion |
| deactivase | | | or mutation |
| aldehyde dehydrogenase A, | 1.2.1.21 1.2.1.22 | aldA | Decrease via deletion |
| NAD-linked | | | or mutation |
| acetaldehyde | 1.2.1.4 | aldB | Decrease via deletion |
| dehydrogenase | | | or mutation |
| Lambda phage DE3 lysogen | N/A | λDE3 | Increase |
| T7 mRNA polymerase | N/A | T7pol | Increase |
| trigger factor | 5.2.1.8 | tig | Decrease via deletion |
| | | | or mutation |
| 3-ketoacyl-CoA thiolase | 2.3.1.16 | fadA | Increase |
| dodecenoyl-CoA δ-isomerase, | 5.3.3.8 1.1.1.35 | fadB | Increase |
| enoyl-CoA hydratase, | 5.1.2.3 4.2.1.17 | | |
| 3-hydroxybutyryl-CoA epimerase, | | | |
| 3-hydroxyacyl-CoA dehydrogenase | | | |
| Sucrose permease | N/A | cscB | Increase |
| Invertase | 3.2.1.26 | cscA | Increase |
| fructokinase | 2.7.1.4 | cscK | Increase |
| carbonic anhydrase | 4.2.1.1 | cynT | Increase |
| carbonic anhydrase | 4.2.1.1 | can | Increase |
| pyridine nucleotide | 1.6.1.2 | pntAB | Increase |
| transhydrogenase | | | |
| pyridine nucleotide | 1.6.1.1 | udhA | Increase |
| transhydrogenase | | | |
| acyl-CoA thioesterase | 3.1.2.20 3.1.2.2 | yciA | Increase and or |
| | | | decrease |
| thioesterase II | 3.1.2.20 3.1.2.2 | tesB | Increase and or |
| | | | decrease |
| thioesterase III | 3.1.2.— | fadM | Increase and or |
| | | | decrease |
| hydroxyphenylacetyl-CoA | N/A | paaI | Increase and or |
| thioesterase | | | decrease |
| esterase/thioesterase | 3.1.2.28 | ybgC | Increase and or |
| | | | decrease |
| proofreading thioesterase in | | entH | Increase and or |
| enterobactin biosynthesis | | | decrease |
| acetoacetyl-CoA synthase | 2.3.1.194 | npth07 | Increase |
| 3-ketoacyl-CoA synthase/elongase | 2.3.1 | Elo1 | Increase |
| 3-ketoacyl-CoA synthase/elongase | 2.3.1 | Elo2 | Increase |
| 3-Hydroxybutyryl-CoA dehydrogenase | 1.1.1.157 | hbd | Increase |
| 3-oxoacyl-CoA reductase | 1.1.1.100 | fabG | Increase |
| enoyl-CoA hydratase | 4.2.1.17 | crt | Increase |
| enoyl-CoA hydratase | 4.2.1.17 | ech2 | Increase |
| Trans-2-enoyl-reductase | 1.3.1.9 | ter | Increase |
| thioesterase | 3.1.2.20 | paaI | Decrease |

E.C. No. = “Enzyme Commission number”

The cell according to any aspect of the present invention may be further genetically modified to comprise a fourth genetic mutation to increase the expression of at least one transporter protein compared to the wild type cell. The transporter protein may be FadL and AlkL. In one example, only FadL or AlkL may be expressed/overexpressed, relative to the wild type cell, in the cell according to any aspect of the present invention. In another example, both FadL and AlkL may be expressed/overexpressed relative to the wild type cell in the cell according to any aspect of the present invention.

The term “cell”, as used herein, refers to any permanently unicellular organism comprising bacteria archaea, fungi, algae and the like. In particular, the cell used according to any aspect of the present invention may be may a bacterial cell. More in particular, the cell may be selected from the group consisting of Pseudomonas, Corynebacterium, Bacillus and Escherichia. Even more in particular, the cell may be Escherichia coli. In one example, the cell may be a lower eukaryote, in particular a fungus from the group consisting of Saccharomyces, Candida, Pichia, Schizosaccharomyces and Yarrowia, in particular, Saccharomyces cerevisiae. The microorganism may be an isolated cell, in other words a pure culture of a single strain, or may comprise a mixture of at least two strains. Biotechnologically relevant cells are commercially available, for example from the American Type Culture Collection (ATCC) or the German Collection of Microorganisms and Cell Cultures (DSMZ). Particles for keeping and modifying cells are available from the prior art, for example Sambrook/Fritsch/Maniatis (1989), and the like. The phrase “in comparison to its wild type has an increased or decreased activity” means that the microorganism has been genetically modified so as to have this increased or decreased activity respectively. A skilled person would understand that either an over-expression of an enzyme in the cell or an expression of an exogenous enzyme may be applicable.

Any aspect of the present invention may be practiced using wild type cells or recombinant cells. In particular, at least one of the enzymes mentioned according to any aspect of the present invention, in particular at least one or all from the group consisting of amino acid N-acyl-transferase, acyl-CoA synthetase acyl-CoA thioesterase an enzyme selected from the group consisting of an enzyme of the glycine cleavage system, glycine hydroxymethyltransferase (GlyA), threonine aldolase (LtaE) threonine dehydrogenase (Tdh), 2-Amino-3-Ketobutyrate CoA-Ligase (Kbl), and/or allothreonine dehydrogenase (YdfG) may be recombinant. The term “recombinant” as used herein, refers to a molecule or is encoded by such a molecule, for example a polypeptide or nucleic acid that, as such, does not occur naturally but is the result of genetic engineering or refers to a cell that comprises a recombinant molecule. For example, a nucleic acid molecule is recombinant if it comprises a promoter functionally linked to a sequence encoding a catalytically active polypeptide and the promoter has been engineered such that the catalytically active polypeptide is overexpressed relative to the level of the polypeptide in the corresponding wild type cell that comprises the original unaltered nucleic acid molecule.

Whether or not a nucleic acid molecule, polypeptide, more specifically an enzyme required according to any aspect of the present invention is recombinant or not may not necessarily affect the level of its expression. However according to any aspect of the present invention, one or more recombinant nucleic acid molecules, polypeptides or enzymes may be required to allow the enzyme to result in the desired increase or decrease in the enzyme expression. In one example, the term “overexpressed”, as used herein, means that the respective polypeptide encoded or expressed is expressed at a level higher or at higher activity than would normally be found in the cell under identical conditions in the absence of genetic modifications carried out to increase the expression, for example in the respective wild type cell. The person skilled in the art is familiar with numerous ways to bring about overexpression. For example, the nucleic acid molecule to be overexpressed or encoding the polypeptide or enzyme to be overexpressed may be placed under the control of a strong inducible promoter such as the lac promoter. The state of the art describes standard plasmids that may be used for this purpose, for example the pET system of vectors exemplified by pET-3a (commercially available from Novagen). Whether or not a nucleic acid or polypeptide is overexpressed may be determined by way of quantitative PCR reaction in the case of a nucleic acid molecule, SDS polyacrylamide electrophoreses, Western blotting or comparative activity assays in the case of a polypeptide. Genetic modifications may be directed to transcriptional, translational, and/or post-translational modifications that result in a change of enzyme activity and/or selectivity under selected and/or identified culture conditions. Thus, in various examples of the present invention, to function more efficiently, a microorganism may comprise one or more gene deletions. Gene deletions may be accomplished by mutational gene deletion approaches, and/or starting with a mutant strain having reduced or no expression of one or more of these enzymes, and/or other methods known to those skilled in the art.

In one example, at least one or all from the group consisting of N-acyl-transferase, acyl-CoA synthetase, acyl-CoA thioesterase, an enzyme of the glycine cleavage system, glycine hydroxymethyltransferase (GlyA) threonine aldolase (LtaE), threonine dehydrogenase (Tdh), 2-Amino-3-Ketobutyrate CoA-Ligase (Kbl), and allothreonine dehydrogenase (YdfG) may be an isolated enzyme. In any event, any enzyme according to any aspect of the present invention may be used in an active state and in the presence of all cofactors, substrates, auxiliary and/or activating polypeptides or factors essential for its activity. The term “isolated”, as used herein, means that the enzyme of interest is enriched compared to the cell in which it occurs naturally. Whether or not an enzyme is enriched may be determined by SDS polyacrylamide electrophoresis and/or activity assays. For example, the enzyme of interest may constitute more than 5, 10, 20, 50, 75, 80, 85, 90, 95 or 99 percent of all the polypeptides present in the preparation as judged by visual inspection of a polyacrylamide gel following staining with Coomassie blue dye.

The cell according to any aspect of the present invention with increased expression of amino acid-N-acyl-transferase and an acyl-CoA synthetase and a decreased expression of at least one enzyme selected from the group consisting of an enzyme of the glycine cleavage system, glycine hydroxymethyltransferase (GlyA) threonine aldolase (LtaE), threonine dehydrogenase (Tdh), 2-Amino-3-Ketobutyrate CoA-Ligase (Kbl), and allothreonine dehydrogenase (YdfG) optionally having a reduced fatty acid degradation capacity may be capable of making proteinogenic amino acids and/or fatty acids. In one example, the term “proteinogenic amino acid”, as used herein, refers to an amino acid selected from the group comprising alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine. Proteinogenic amino acids and fatty acids are synthesized as part of the primary metabolism of many wild type cells in reactions and using enzymes that have been described in detail in biochemistry textbooks, for example Jeremy M Berg, 2002.

In one example, the cell according to any aspect of the present invention expresses an acyl-CoA thioesterase. In particular, the term “acyl-CoA thioesterase”, as used herein, refers to an enzyme capable of hydrolyzing acyl-CoA. In one example, the acyl-CoA thioesterase comprises a sequence from the group consisting of SEQ ID NO:1, AEM72521.1 and AAC49180.1 or a variant thereof. In particular, the acyl-CoA thioesterase comprises SEQ ID NO:1. The activity of acyl-CoA thioesterase may be assayed using various assays described in the state of the art. Briefly, the reaction of Ellman's reagent, which reacts with free thiol groups associated with CoASH formed upon hydrolysis of acyl-CoA may be detected by spectophotometrically monitoring absorbance at 412 nm.

According to another aspect of the present invention, there is provided a method for producing acyl amino acids, comprising contacting an amino acid and a fatty acid and/or an acyl CoA thereof in the presence of at least one cell according to any aspect of the present invention. The amino acid-N-acyl-transferase, may be isolated and/or recombinant wherein the amino acid-N-acyl-transferase may be a human amino acid-N-acyl-transferase, in particular of SEQ ID NO:63, SEQ ID NO:64 or a variant thereof, wherein the acyl-CoA synthetase may be in particular SEQ ID NO:65 or a variant thereof, the GcvT, GcvP and GcvH may be SEQ ID NO:58, SEQ ID NO:60 and SEQ ID NO:59 respectively, the GlyA may be SEQ ID NO:61 and the LtaE may be SEQ ID NO:62 or culturing the cell according to any aspect of the present invention.

The method according to any aspect of the present invention my further comprise additional steps of
converting a fatty acid to an acyl CoA using an acyl-CoA synthetase, which may be isolated and/or recombinant; and/or
hydrogenating.

All the enzymes according to any aspect of the present invention, in particular selected from the group consisting of amino acid-N-acyl-transferase, acyl-CoA synthetase, GcvT, GcvP, GcvH, GlyA, LtaE, Tdh, Kbl, and YdfG may be provided in the form of a cell expressing said enzyme or enzymes.

The term “contacting”, as used herein, means bringing about direct contact between the amino acid, the acyl CoA and the cell according to any aspect of the present invention. In one example, the amino acid, the acyl CoA and the cell according to any aspect of the present invention are brought together in an aqueous solution. For example, the cell, the amino acid and the acyl CoA may not be in different compartments separated by a barrier such as an inorganic membrane. If the amino acid or fatty acid is soluble and may be taken up by the cell or can diffuse across biological membranes, it may simply be added to the cell according to any aspect of the present invention in an aqueous solution. In case it is insufficiently soluble, it may be dissolved in a suitable organic solvent prior to addition to the aqueous solution. The person skilled in the art is able to prepare aqueous solutions of amino acids or fatty acids having insufficient solubility by adding suitable organic and/or polar solvents. Such solvents may be provided in the form of an organic phase comprising liquid organic solvent. In one example, the organic solvent or phase may be considered liquid when liquid at 25° C. and standard atmospheric pressure. In another example, the fatty acid may be provided in the form of a fatty acid ester such as the respective methyl or ethyl ester. For example, the fatty acid laurate may be dissolved in lauric acid methyl ester as described in EP11191520.3. According to any aspect of the present invention, the compounds and catalysts may be contacted in vitro, i.e. in a more or less enriched or even purified state, or may be contacted in situ, i.e. they are made as part of the metabolism of the cell and subsequently react inside the cell.

The term “an aqueous solution” comprises any solution comprising water, in particular mainly water as solvent that may be used to keep the cell according to any aspect of the present invention, at least temporarily, in a metabolically active and/or viable state and comprises, if such is necessary, any additional substrates. The person skilled in the art is familiar with the preparation of numerous aqueous solutions, usually referred to as media that may be used to keep the cells according to any aspect of the present invention, for example LB medium in the case of E. coli. It is advantageous to use as an aqueous solution a minimal medium, i.e. a medium of reasonably simple composition that comprises only the minimal set of salts and nutrients indispensable for keeping the cell in a metabolically active and/or viable state, by contrast to complex mediums, to avoid dispensable contamination of the products with unwanted side products. For example, M9 medium may be used as a minimal medium.

Advantageously, the cell and method according to any aspect of the present invention may use not only saturated fatty acids, but also unsaturated fatty acids may be converted to acyl amino acids. In case the end product sought-after is to comprise a higher yield of saturated acyl residues than is present at the beginning of the method according to any aspect of the present invention, it may be possible to complement the process by hydrogenating the acyl residues of the acyl amino acids. The hydrogenation may be carried out according to various state of the art processes, for example those described in U.S. Pat. No. 5,734,070. Briefly, the compound to be hydrogenated may be incubated at 100° C. in the presence of hydrogen and a suitable catalyst, for example a nickel catalyst on silicon oxide as a support.

According to another aspect of the present invention, there is provided a reaction mixture comprising
an amino acid-N-acyl-transferase, which may be isolated and/or recombinant,
an acyl-CoA synthetase, which may be isolated and/or recombinant,
an amino acid and/or
either an acyl CoA or a fatty acid and an acyl-CoA-synthetase,
wherein the amino acid-N-acyl-transferase may be a human amino acid-N-acyl-transferase, in particular of SEQ ID NO: 4, SEQ ID NO: 5 or a variant thereof,
wherein the acyl-CoA synthetase may be SEQ ID NO: 6 or a variant thereof,
wherein the GcvT, GcvP and GcvH may be SEQ ID NO:58, SEQ ID NO:60 and SEQ ID NO:59 or a variant thereof respectively,
wherein the GlyA may be SEQ ID NO:61;
wherein the LtaE may be SEQ ID NO:62;
wherein the Tdh may be SEQ ID NO:81;
wherein the Kbl may be SEQ ID NO:80; and
wherein the YdfG may be SEQ ID NO:82.

According to a further aspect of the present invention, there is provided a composition comprising
a first acyl amino acid consisting of a saturated acyl having 8 to 16 carbon atoms and glycine,
a second acyl amino acid consisting of an unsaturated acyl having 10 to 18 carbon atoms and glycine,
and optionally a third acyl amino acid consisting of a saturated or unsaturated acyl having 12 carbon atoms and an amino acid selected from the group consisting of glutamine, glutamic acid, alanine and asparagine.

The composition according to any aspect of the present invention may comprise a first acyl amino acid which comprises a saturated acyl having 12 carbon atoms, in particular lauryl, and glycine. The second acyl amino acid may comprise an unsaturated acyl having 12 or 14 carbon atoms and glycine. In particular, the acyl amino acids formed may be a mixture of amino acids. The mixture of amino acids may comprise at least two proteinogenic amino acids as defined below. In particular, the mixture of amino acids may have 3, 4, 5, 6, 7, 8, 9, 10, 11, 12, 13, 14, 15, 16, 17, 18, 19, 20, or 21 proteinogenic amino acids. In one example, the mixture of amino acids may be used to form cocoyl glycine and salts thereof.

Advantageously, the composition of acyl amino acids produced according to any aspect of the present invention, more specifically the length of fatty acids incorporated into such acyl amino acids, may be controlled by introducing into the cell one or more specific acyl-CoA thioesterases or altering the expression of one or more acyl-CoA thioesterases endogenously expressed by the cell.

### BRIEF DESCRIPTION OF FIGURES

The inventions are further illustrated by the following figures and non-limiting examples from which further embodiments, aspects and advantages of the present invention may be taken.

FIG. 1 is a graph depicting a total ion chromatogram of the 48 h sample from the E. coli W3110 ΔfadE pJ294{Ptac}[synUcTE]/pCDF{Ptac}[hGLYAT2(co_Ec)_fadD_Ec] fermentation.

FIG. 2 is a graph depicting a total ion chromatogram of the 48 h sample from the E. coli W3110 ΔfadE pJ294{Ptac}[synUcTE]/pCDF{Ptac}[hGLYAT3(co_Ec)_fadD_Ec] fermentation.

FIG. 3 is a graph depicting a total ion chromatogram of the 48 h sample from the E. coli W3110 ΔfadE pJ294{Ptac}[synUcTE]/pCDFDuet-1 fermentation (negative control).

FIG. 4 is a graph showing the production of lauroylglycinate by E. coli strains W3110 ΔfadE pCDF{Ptac}[hGLYAT2(co_Ec)-fadD_Ec]{Placuv5}[alkLmod1].

FIG. 5 is a graph showing the production of lauroylglycinate by E. coli strain W3110 ΔfadE ΔgcvTHP pCDF{Ptac}[hGLYAT2(co_Ec)-fadD_Ec]{Placuv5}[alkLmod1].

FIG. 6 is a tree showing the percentage sequence identity of GLYAT2 in the various organisms tested in Example 18.

FIG. 7 is an illustration showing the metabolic pathways of glycine.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Sequence ID NOs:

Throughout this application a range of SEQ ID NOs are used. These are shown in Table 3 below.

TABLE 3
Sequences used in the Examples.

| SEQ ID | |
|---|---|
| NO: | Comment |
| 1 | Umbellularia californica synUcTE (an acyl-CoA thioesterase) |
| | gene (codon-optimized) |
| 2 | tac promoter |
| 3 | Vector pJ294[Ptac-synUcTE], see example 1 |
| 4 | Homo sapiens genes hGLYAT2 (an amino acid N-acyl |
| | transferase) |
| 5 | Homo sapiens genes hGLYAT3 (another amino acid N-acyl |
| | transferase) |
| 6 | Escherichia coli fadD (an acyl-CoA synthetase) |
| 7 | Vector pCDF[atfA1_Ab(co_Ec)-fadD_Ec], see example 2 |
| 8 | Vector pCDF{Ptac}[hGLYAT2(co_Ec)-fadD_Ec], see example 2 |
| 9 | Vector pCDF{Ptac}[hGLYAT3(co_Ec)-fadD_Ec], see example 2 |
| 10 | alkL (an importer facilitating transport of hydrophobic |
| | acyl across cell membranes) gene, see example 3 |
| 11 | lacuv5 promoter, see example 3 |
| 12 | Vector pCDF[alkLmod1], see example 3 |
| 13 | Vector pCDF{Ptac}[hGLYAT2(co_Ec)-fadD_Ec]{Placuv5}[alkLmod1], |
| | see example 3 |
| 14 | Vector pET-28b, see example 10 |
| 15 | Vector pET-28b{Ptac}[hGLYAT2(co_Ec)], see example 10 |
| 16 | pET-28b{Ptac}[hGLYAT3(co_Ec)], see example 10 |

### Example 1

Generation of an Expression Vector for the Umbellularia californica Gene synUcTE

To generate an expression vector for the Umbellularia californica synUcTE gene (SEQ ID NO:1), which encodes the Umbellularia californica acyl CoA-thioesterase, this gene was codon-optimized for expression in Escherichia coli. The gene was synthesized together with a tac promoter (SEQ ID N0:2), and, simultaneously, one cleavage site was introduced upstream of the promoter and one cleavage site downstream of the terminator. The synthesized DNA fragment Ptac-synUcTE was digested with the restriction endonucleases BamHI and NotI and ligated into the correspondingly cut vector pJ294 (DNA2.0 Inc., Menlo Park, Calif., USA). The finished E. coli expression vector was referred to as pJ294[Ptac-synUcTE] (SEQ ID NO:3).

### Example 2

Generation of Vectors for Coexpression of Escherichia coli fadD with Either the Homo sapiens Genes hGLYAT3 and hGLYAT2

To generate vectors for the coexpression of the Homo sapiens genes hGLYAT2 (SEQ ID NO:4) or hGYLAT3 (SEQ ID NO:5), which encodes human glycine-N-acyltransferase, with Escherichia coli fadD (SEQ ID NO:6), which encodes the E. coli acyl-CoA synthetase, the genes hGLYAT2 and hGLYAT3 were codon-optimized for expression in Escherichia coli and synthesized. The synthesized DNA fragments were digested with the restriction endonucleases SacII and Eco47III and ligated into the correspondingly cut pCDF[atfA1_Ab(co_Ec)-fadD_Ec] (SEQ ID NO:7) with removal of the aftAl gene. The sequence segments which were additionally removed in this process were cosynthesized during gene synthesis. The vector is a pCDF derivative which already comprises a synthetic tac promoter (SEQ ID NO:2) and the Escherichia coli fadD gene. The resulting expression vectors were named pCDF{Ptac}[hGLYAT2(co_Ec)-fadD_Ec] (SEQ ID NO:8) and pCDF{Ptac}[hGLYAT3(co_Ec)-fadD_Ec] (SEQ ID NO:9).

### Example 3

Generation of Vectors for the Coexpression of the Homo sapiens hGLYAT2, Escherichia coli fadD and Pseudomonas putida alkL Genes

To generate vectors for the coexpression of the hGLYAT2 genes with a modified Pseudomonas putida alkL gene, which encodes AlkL, an outer membrane protein that facilitates the import of hydrophobic substrates into a cell, the alkL gene (SEQ ID NO:10) was amplified together with the lacuv5 promoter (SEQ ID NO:11) from the plasmid pCDF[alkLmod1] (SEQ ID NO:12) by means of sequence-specific oligonucleotides. The PCR products were cleaved with the restriction endonucleases BamHI and NsiI and ligated into the correspondingly cleaved vector pCDF{Ptac}[hGLYAT2(co_Ec)-fadD_Ec] (SEQ ID NO:8). The correct insertion of the target genes was checked by restriction analysis and the authenticity of the introduced genes was verified by DNA sequencing. The resulting expression vector was named pCDF{Ptac}[hGLYAT2(co_Ec)-fadD_Ec]{Placuv5}[alkLmod1] (SEQ ID NO:13).

The following parameters were used for PCR: 1×: initial denaturation, 98° C., 3:00 min; 35× denaturation, 98° C., 0:10 min; annealing, 65° C., 0:20 min; elongation, 72° C., 0:17 min; 1×: final elongation, 72° C., 10 min. For amplification the Phusion™ High-Fidelity Master Mix from New England Biolabs (Frankfurt) was used according to manufacturer's manual. 50 μl of the PCR reaction were analyzed on a 1% TAE agarose gel. Procedure of PCR, agarose gel electrophoresis, ethidium bromide staining of DNA and determination of PCR fragment size were carried out known to those skilled in the art.

### Example 4

Generation of an E. coli Strain with Deletion in the fadE Gene, which Strain Overexpresses the Umbellularia californica synUcTE, Escherichia coli fadD and Homo sapiens hGLYAT2 and hGLYAT3 Genes

To generate E. coli strains which coexpress the Umbellularia californica synUcTE in combination with the Escherichia coli fadD and Homo sapiens hGLYAT2 or Homo sapiens hGLYAT3 genes, the strain E. coli W3110 ΔfadE was transformed with the plasmids pJ294{Ptac}[synUcTE] and pCDF{Ptac}[hGLYAT2(co_Ec)_fadD_Ec] or pCDF{Ptac}[hGLYAT3(co_Ec)_fadD_Ec] by means of electroporation and plated onto LB-agar plates supplemented with spectinomycin (100 μg/ml) and ampicillin (100 μg/ml). Transformants were checked for the presence of the correct plasmids by plasmid preparation and analytic restriction analysis. The strains E. coli W3110 ΔfadE pJ294{Ptac}[synUcTE]/pCDF{Ptac}[hGLYAT2(co_Ec)_fadD_Ec] and E. coli W3110 ΔfadE pJ294{Ptac}[synUcTE]/pCDF{Ptac}[hGLYAT3(co_Ec)_fadD_Ec] were generated thus.

### Example 5

Generation of an E. coli Strain with Deletion in the fadE Gene, which Strain Overexpresses the Escherichia coli fadD and Either Homo sapiens hGLYAT2 or hGLYAT3 Genes

To generate E. coli strains which overexpress the Escherichia coli fadD gene in combination with the Homo sapiens hGLYAT2 or hGLYAT3 genes, electrocompetent cells of E. coli strain W3110 ΔfadE were generated. E. coli W3110 ΔfadE was transformed with the plasmid pCDF{Ptac}[hGLYAT2(co_Ec)-fadD_Ec]{Placuv5}[alkLmod1] and plated onto LB-agar plates supplemented with spectinomycin (100 μg/ml). Transformants were checked for the presence of the correct plasmids by plasmid preparation and analytic restriction analysis. The strain generated thus was named E. coli W3110 ΔfadE pCDF{Ptac}[hGLYAT2(co_Ec)-fadD_Ec]{Placuv5}[alkLmod1].

### Example 6

Production of Fatty Acid/Amino Acid Adducts by E. coli Strains with Deletion in the fadE Gene, which Strains Overexpress the synUcTE and fadD Genes in Combination with Either hGLYAT2 or hGLYAT3

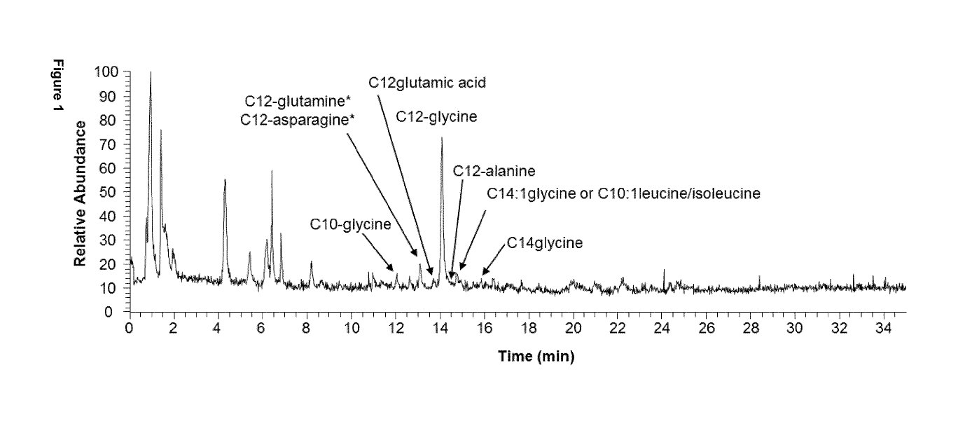

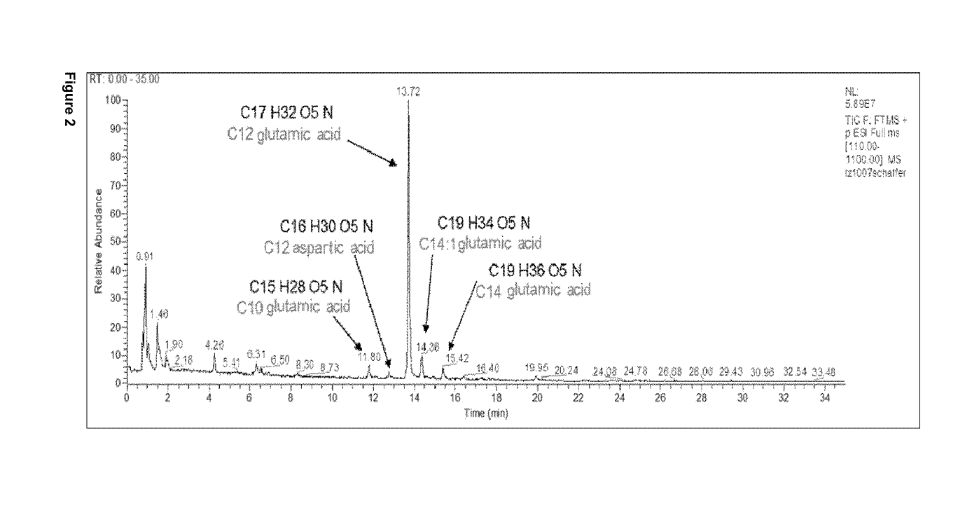

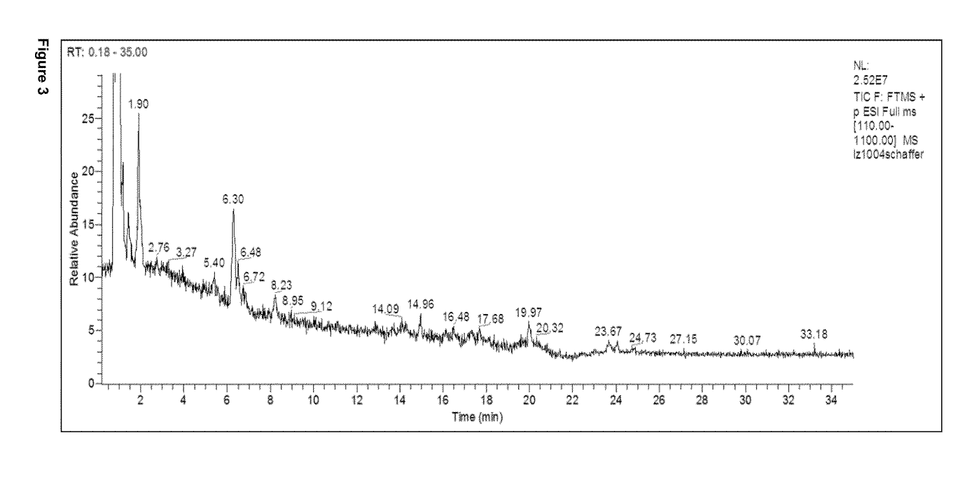

The strains generated in Example 4 were used to study their ability to produce fatty acid/amino acid adducts. Starting from a −80° C. glycerol culture, the strains to be studied were first plated onto an LB-agar plate supplemented with 100 μg/ml ampicillin and 100 μg/ml spectinomycin and incubated overnight at 37° C. Starting from a single colony in each case, the strains were then grown as a 5-ml preculture in Luria-Bertani broth, Miller (Merck, Darmstadt) supplemented with 100 μg/mlampicillin and 100 μg/ml spectinomycin. The further culture steps were performed in M9 medium. The medium, composed of 38 mM disodium hydrogenphosphate dihydrate, 22 mM potassium dihydrogenphosphate, 8.6 mM sodium chloride, 37 mM ammonium chloride, 2% (w/v) glucose, 2 mM magnesium sulphate heptahydrate (all chemicals from Merck, Darmstadt) and 0.1% (v/v) trace element solution, was brought to pH 7.4 with 25% strength ammonium hydroxide solution. The trace element solution added, composed of 9.7 mM manganese(II) chloride tetrahydrate, 6.5 mM zinc sulphate heptahydrate, 2.5 mM sodium-EDTA (Titriplex III), 4.9 mM boric acid, 1 mM sodium molybdate dihydrate, 32 mM calcium chloride dihydrate, 64 mM iron(II) sulphate heptahydrate and 0.9 mM copper(II) chloride dihydrate, dissolved in 1 M hydrochloric acid (all chemicals from Merck, Darmstadt) was filter-sterilized before being added to the M9 medium. 20 ml of M9 medium supplemented with 100 μg/ml spectinomycin and 100 μg/mlampicillin were introduced into baffled 100-ml Erlenmeyer flasks and inoculated with 0.5 ml preculture. The flasks were cultured at 37° C. and 200 rpm in a shaker-incubator. After a culture time of 8 hours, 50 ml of M9 medium supplemented with 100 μg/ml spectinomycin and 100 μg/mlampicillin were introduced into a baffled 250-ml Erlenmeyer flask and inoculated with the 10-ml culture to achieve an optical density (600 nm) of 0.2. The flasks were cultured at 37° C. and 200 rpm in a shaker-incubator. When an optical density (600 nm) of 0.7 to 0.8 was reached, gene expression was induced by addition of 1 mM IPTG. The strains were cultured for a further 48 hours at 30° C. and 200 rpm. Simultaneously with the induction, 1 g/l glycine was added to some of the cultures. During culturing, samples were taken, and fatty acid/amino acid adducts present were analysed. The results are shown in FIGS. 1 and 2. It has been possible to demonstrate that both E. coli strain W3110 ΔfadE pJ294{Ptac}[synUcTE]/pCDF{Ptac}[hGLYAT2(co_Ec)_fadD_Ec] and E. coli strain W3110 ΔfadE pJ294{Ptac}[synUcTE]/pCDF{Ptac}[hGLYAT3(co_Ec)_fadD_Ec] are capable of forming various fatty acid/amino acid adducts, for example lauroyl-glutamic acid, from glucose. By contrast, no such adducts can be found in a cell that, as a negative control, lacks the plasmids (FIG. 3). It appears that slight sequence variations, for example amino acid substitutions that distinguish hGLYAT2 from hGLYAT3, do not compromise the ability of the cell to make the fatty acid/acyl amino acid adducts as shown in Table 4.

TABLE 4
Quantitative determination of fatty acid glycinates after 48 h culture time

| | C_{lauroylglycinate} | C_{myristoylglycinate} |
|---|---|---|
| Strain | [mg/L] | [mg/L] |
| E. coli W3110 ΔfadE pJ294{Ptac}[synUcTE]/ | 111 | <2 |
| pCDF{Ptac}[hGLYAT2(co_Ec)_fadD_Ec] | | |
| E. coli W3110 ΔfadE pJ294{Ptac}[synUcTE]/ | 121 | 2.8 |
| pCDF{Ptac}[hGLYAT2(co_Ec)_fadD_Ec] + | | |
| 1 g/L glycine | | |
| E. coli W3110 ΔfadE pJ294{Ptac}[synUcTE]/ | n.d. | n.d. |
| pCDFDuet-1 | | |

### Example 7

### Chromatographic Quantification of Products by HPLC/MS

The quantification of N-lauroylglycine, N-myristoylglycine and N-palmitoylglycine and the detection of other acylamino acids in fermentation samples was performed by HPLC-ESI/MS. The quantification was performed with the aid of an external calibration (approx. 0.1-50 mg/l) for the three target compounds in the “single ion monitoring” mode (SIM). In parallel, a scan was carried out over a mass range m/z=100-1000 so as to identify further acylamino acids.

The samples for the determination of the fatty acid glycinates were prepared as follows: 800 μl of solvent (acetone) and 200 μl of sample were pipetted into a 2-ml reaction vessel. The mixture was shaken in a Retsch mill for 1 minute at 30 Hz and then centrifuged for 5 min at approximately 13 000 rpm. The clear supernatant was removed using a pipette and, after suitable dilution with diluent (80% acetonitrile/20% water+0.1% formic acid), analyzed. The calibration standards used were likewise dissolved and diluted in this diluent.

The following equipment was employed:
Surveyor HPLC system (Thermo Scientific, Waltham, Mass., USA) composed of MS pump, Autosampler Plus and PDA Detector Plus
Mass spectrometer TSQ Vantage with HESI II source (Thermo Scientific, Waltham, Mass., USA)
HPLC column: 100×2 mm Pursuit XRS Ultra C8; 2.8 μm (Agilent, Santa Clara, Calif., USA)

### Chemicals:

Water from a Millipore system
Acetonitrile for HPLC (Merck AG, Darmstadt, Germany)
Formic acid, p.a. grade (Merck, Darmstadt, Germany)
N-propanol Lichrosolv (Merck, Darmstadt, Germany)
N-lauroylglycine 99% (Chem-Impex International, Wood Dale, Ill., USA)
N-myristoylglycine >98% (Santa Cruz Biotechnology, Texas, USA)
N-palmitoylglycine >99% (provenance unknown)

The HPLC separation was carried out using the abovementioned HPLC column. The injection volume amounted to 2 μl, the column temperature to 40° C., the flow rate to 0.3 ml/min. The mobile phase consisted of Eluent A (0.1% strength (v/v) aqueous formic acid) and Eluent B (75% acetonitrile/25% n-propanol (v/v) with 0.1% (v/v) formic acid). The following gradient profile was used:

TABLE 5
Gradient profile used in Example 7.

| Time | Eluent A | Eluent B |
|---|---|---|
| [min] | [%] | [%] |
| 0 | 90 | 10 |
| 1 | 90 | 10 |
| 20 | 5 | 95 |
| 25 | 5 | 95 |

The HPLC/MS analysis was carried out under positive ionization mode with the following parameters of the ESI source:

| Spray Voltage: | 3500 | V |
|---|---|---|
| Vaporizer Temperature: | 50° | C. |
| Sheath Gas Pressure: | 40 | |
| Aux. Gas Pressure: | 10 | |
| Capillary Temperature: | 250° | C. |

| | |
|---|---|
| Sprayer Distance: | Ring C |

Detection and quantification of the three analytes were performed by “single ion monitoring” (SIM) with the following parameters shown in Table 6.

TABLE 6
Parameters used in SIM of Example 7.

| | Ion [M + H] | Scan range | Scan time | Resolution |
|---|---|---|---|---|
| Analyte | [m/z] | [m/z] | [ms] | Q3 |
| N-lauroylglycine | 258.2 | 0.002 | 50 | 0.7 |
| N-myristoylglycine | 286.2 | 0.002 | 50 | 0.7 |
| N-palmitoylglycine | 314.2 | 0.002 | 50 | 0.7 |

### Example 8

Production of Fatty Acid Amino Acid Adducts by E. coli Strains with Deletion in the fadE Gene, which Strains Overexpress the synUcTE and fadD Genes in Combination with hGLYAT2 or hGLYAT3 in a Parallel Fermentation System

The strains generated in Example 4 were used for studying their ability to produce fatty acid amino acid adducts from glucose. For this purpose, the strain was cultured both in a shake flask and in a fed-batch fermentation. The fermentation was carried out in a parallel fermentation system from DASGIP with 8 bioreactors.

The production cells were prepared as described in Example 6.

The fermentation was performed using 1 l reactors equipped with overhead stirrers and impeller blades. pH and pO₂ were measured online for process monitoring. OTR/CTR measurements served for estimating the metabolic activity and cell fitness, inter alia.

The pH electrodes were calibrated by means of a two-point calibration using standard solutions of pH 4.0 and pH 7.0, as specified in DASGIP's technical instructions. The reactors were provided with the necessary sensors and connections as specified in the technical instructions, and the agitator shaft was fitted. The reactors were then charged with 300 ml of water and autoclaved for 20 min at 121° C. to ensure sterility. The pO₂ electrodes were connected to the measuring amplifiers and polarized overnight (for at least 6 h). Thereafter, the water was removed under a clean bench and replaced by M9 medium (pH 7.4) composed of KH₂PO₄ 3.0 g/l, Na₂HPO₄ 6.79 g/l, NaCl 0.5 g/l, NH₄Cl 2.0 g/l, 2 ml of a sterile 1 M MgSO₄*7H₂O solution and 1 ml/l of a filter-sterilized trace element stock solution (composed of HCl (37%) 36.50 g/l, MnCl₂*4H₂O 1.91 g/l, ZnSO₄*7H₂O 1.87 g/l, ethylenediaminetetraacetic acid dihydrate 0.84 g/l, H₃BO₃ 0.30 g/l, Na₂MoO₄*2H₂O 0.25 g/l, CaCl₂*2H₂O 4.70 g/l, FeSO₄*7H₂O 17.80 g/l, CuCl₂*2H₂O 0.15 g/l) with 15 g/l glucose as the carbon source (added by metering in 30 ml/l of a sterile feed solution composed of 500 g/l glucose, 1.3% (w/v) MgSO₄*7H₂O) supplemented with 100 mg/l spectinomycin and 3 ml/l DOW1500.

Thereafter, the pO₂ electrodes were calibrated to 100% with a one-point calibration (stirrer: 400 rpm/aeration: 10 sl/h air), and the feed, correction agent and induction agent lines were cleaned by “cleaning in place” as specified in the technical instructions. To this end, the tubes were rinsed first with 70% ethanol, then with 1 M NaOH, then with sterile fully-demineralized water and, finally, filled with the respective media.

Using the E. coli strain of Example 4, a dilution streak was first performed with a cryoculture on an LB agar plate supplemented with 100 mg/l spectinomycin, and the plate was incubated for approximately 16 h at 37° C. LB medium (10 ml in a 100-ml baffle flask) supplemented with 100 mg/l spectinomycin was then inoculated with a single colony and the culture was grown overnight at 37° C. and 200 rpm for approximately 16 h. Thereafter, this culture was used for a second preculture stage with an initial OD of 0.2 in 50 ml of M9 medium, composed of KH₂PO₄ 3.0 g/l, Na₂HPO₄ 6.79 g/l, NaCl 0.5 g/l, NH₄Cl 2.0 g/l, 2 ml of a sterile 1 M MgSO₄*7H₂O solution and 1 ml/l of a filter-sterilized trace element stock solution (composed of HCl (37%) 36.50 g/l, MnCl₂*4H₂O 1.91 g/l, ZnSO₄*7H₂O 1.87 g/l, ethylenediaminetetraacetic acid dihydrate 0.84 g/l, H₃BO₃ 0.30 g/l, Na₂MoO₄*2H₂O 0.25 g/l, CaCl₂*2H₂O 4.70 g/l, FeSO₄*7H₂O 17.80 g/l, CuCl₂*2H₂O 0.15 g/l) supplemented with 20 g/l glucose as carbon source (added by metering in 40 ml/l of a sterile feed solution composed of 500 g/l glucose) together with the above-described antibiotics was transferred into a 500-ml baffle flask and incubated for 8-12 h at 37° C./200 rpm.

To inoculate the reactors with an optical density of 0.1, the 00600 of the second preculture stage was measured and the amount of culture required for the inoculation was calculated. The required amount of culture was placed into the heated and aerated reactor with the aid of a 5-ml syringe through a septum.

The standard program shown in Table 7a-c was used:

TABLE 7
Standard program for use of heated and aerated reactor in Example 8
a)

| | |
|---|---|
| DO controller | pH controller |

| | | | |
|---|---|---|---|
| Preset | 0% | Preset | 0 ml/h |
| P | 0.1 | P | 5 |
| Ti | 300 s | Ti | 200 s |
| Min | 0% | Min | 0 ml/h |
| Max | 100% | Max | 40 ml/h |

b)

| | | | XO2 | | | F | | |
|---|---|---|---|---|---|---|---|---|
| | | | (gas | | | (gas | | |
| N (Rotation) | from | to | mixture) | from | to | flow) | from | to |
| Growth and | 0% | 30% | Growth and | 0% | 100% | Growth and | 15% | 80% |
| biotransformation | 400 rpm | 1500 rpm | biotransformation | 21% | 21% | biotransformation | 6 sl/h | 72 sl/h |

c)

| Script | |
|---|---|
| Trigger fires | 31% DO (1/60 h) |
| Induction | 2 h after the feed |
| IPTG | start |
| Feed trigger | 50% DO |
| Feed rate | 3 [ml/h] |

The pH was adjusted unilaterally to pH 7.0 with 12.5% strength ammonia solution. During the growth phase and the biotransformation, the dissolved oxygen (pO₂ or DO) in the culture was adjusted to at least 30% via the stirrer speed and the aeration rate. After the inoculation, the DO dropped from 100% to these 30%, where it was maintained stably for the remainder of the fermentation.

The fermentation was carried out as a fed batch, the feed start as the beginning of the feed phase with 5 g/l*h glucose feed, composed of 500 g/l glucose, 1.3% (w/v) MgSO₄*7H₂O, being triggered via the DO peak which indicates the end of the batch phase. From the feed start onwards, the temperature was reduced from 37° C. to 30° C. 2 h after the feed start, the expression was induced with 1 mM IPTG.

To quantify lauroyl, myristoyl and palmitoyl glycinate, samples were taken 47 h and 64 h after the start of the fermentation. These samples were prepared for analysis, and analyzed as described in Example 7.

It has been possible to demonstrate that strain E. coli W3110 ΔfadE pJ294{Ptac}[synUcTE]/pCDF{Ptac}[hGLYAT2(co_Ec)_fadD_Ec] is capable of forming lauroyl glycinate from glucose.

TABLE 8
Quantification of fatty acid glycinates
after 47 and 64 h fermentation time.

| | Ion [M + H] | Scan range | Scan time | Resolution |
|---|---|---|---|---|
| Analyte | [m/z] | [m/z] | [ms] | Q3 |
| N-Lauroylglycine | 258.2 | 0.002 | 50 | 0.7 |
| N-Myristoylglycine | 286.2 | 0.002 | 50 | 0.7 |
| N-Palmitoylglycine | 314.2 | 0.002 | 50 | 0.7 |

TABLE 9
Production of fatty acids after 47 and 64 hours' fermentation time. (n.d.: not determined)

| Fermentation | | | | | | |
|---|---|---|---|---|---|---|
| time [h] | C_{Lauroyl glycinate} [g/l] | C_{Myristoyl glycinate} [g/l] | C_{Palmitoyl glycinate} [g/l] | C_{Glycine} [g/l] | C_{Octanoic acid} [g/l] | C_{Decanoic acid} [g/l] |
| 47 | 0.63 | 0.07 | n.d. | n.d. | n.d. | 0.001 |
| 64 | 1.06 | 0.11 | n.d. | n.d. | n.d. | n.d. |

| Fermentation | | | | | | |
|---|---|---|---|---|---|---|
| time [h] | C_{Lauric acid} [g/l] | C_{Myristic acid} [g/l] | C_{Palmitoleic acid} [g/l] | C_{Palmitic acid} [g/l] | C_{Oleic acid} [g/l] | C_{Stearic acid} [g/l] |
| 47 | n.d. | n.d. | n.d. | 0.002 | 0.006 | 0.001 |
| 64 | 0.001 | n.d. | n.d. | n.d. | 0.002 | n.d. |

### Example 9

The strain of Example 5 was fermented in a fed-batch fermentation to study the ability of linking lauric acid and glycine to give lauroyl glycinate. This fermentation was carried out in a parallel fermentation system from DASGIP with 8 bioreactors.

The experimental setting was as described in Example 8 except that 100 g/l glycine in demineralized water and 100 g/l laurate in lauric acid methyl ester were fed rather than glucose. To quantify lauroyl, myristoyl and palmitoyl glycinate in fermentation samples, samples were taken 23 h and 42 h after the start of the fermentation. These samples were prepared for analysis, and analyzed as described in Example 7. The results are shown in Tables 10 and 11.

It has been possible to demonstrate that the strain E. coli W3110 ΔfadE pCDF{Ptac}[hGLYAT2(co_Ec)_fadD_Ec] {Plavuv5} [alkLmod1] is capable of linking lauric acid and glycine and of producing lauroyl glycinate.

TABLE 10
Production of lauroyl glycinate after fermentation for 23 and 42 hours with feeding of lauric acid and glycine.

| Fermentation | | | | | | |
|---|---|---|---|---|---|---|
| time [h] | C_{Lauroyl glycinate} [g/l] | C_{Myristoyl glycinate} [g/l] | C_{Palmitoyl glycinate} [g/l] | C_{Glycine} [g/l] | C_{Octanoic acid} [g/l] | C_{Decanoic acid} [g/l] |
| 21 | 1.02 | n.d. | n.d. | n.d. | n.d. | n.d. |
| 40 | 1.78 | n.d. | n.d. | n.d. | n.d. | n.d. |

| Fermentation | | | | | | |
|---|---|---|---|---|---|---|
| time [h] | C_{Lauric acid} [g/l] | C_{Myristic acid} [g/l] | C_{Palmitoleic acid} [g/l] | C_{Palmitic acid} [g/l] | C_{Oleic acid} [g/l] | C_{Stearic acid} [g/l] |
| 21 | 6.38 | n.d. | n.d. | n.d. | n.d. | n.d. |
| 40 | 6.79 | n.d. | n.d. | n.d. | n.d. | n.d. |

TABLE 11
Production of lauroyl glycinate after fermentation for 23 and 42 hours without feeding of lauric
acid and glycine (negative control).

| Fermentation | | | | | | |
|---|---|---|---|---|---|---|
| time [h] | C_{Lauroyl glycinate} [g/l] | C_{Myristoyl glycinate} [g/l] | C_{Palmitoyl glycinate} [g/l] | C_{Glycine} [g/l] | C_{Octanoic acid} [g/l] | C_{Decanoic acid} [g/l] |
| 21 | n.d. | n.d. | n.d. | n.d. | n.d. | n.d. |
| 40 | n.d. | n.d. | n.d. | n.d. | n.d. | n.d. |

| Fermentation | | | | | | |
|---|---|---|---|---|---|---|
| time [h] | C_{Lauric acid} [g/l] | C_{Myristic acid} [g/l] | C_{Palmitoleic acid} [g/l] | C_{Palmitic acid} [g/l] | C_{Oleic acid} [g/l] | C_{Stearic acid} [g/l] |
| 21 | n.d. | n.d. | n.d. | n.d. | n.d. | n.d. |
| 40 | n.d. | n.d. | n.d. | n.d. | n.d. | n.d. |

### Example 10

Generation of Vectors for Expression of the Homo sapiens Genes hGLYAT3 and hGLYAT2 in E. coli Strains Producing Fatty Acids Via Malonyl-CoA and Acetyl-CoA

To generate vectors for the expression of the Homo sapiens genes hGLYAT2 (SEQ ID NO: 4) or hGYLAT3 (SEQ ID NO: 5) in the fatty acid producing strains listed in Table 3 below (from Table 3.2 of WO2014026162A1), the genes hGLYAT2 and hGYLAT3 were first amplified. The gene hGLYAT2 was amplified from the plasmid pCDF{Ptac}[hGLYAT2(co_Ec)-fadD_Ec]{Placuv5}[alkLmod1] (SEQ ID NO:13) and the hGYLAT3 gene was amplified from the plasmid pCDF{Ptac}[hGLYAT3(co_Ec)-fadD_Ec] (SEQ ID NO:9) by means of sequence-specific oligonucleotides. The PCR products were cleaved with the restriction endonucleases NotI and Sacl and ligated in the correspondingly cleaved vector pET-28b (SEQ ID NO:14). The correct insertion of the target genes was checked by restriction analysis and the authenticity of the introduced genes was verified by DNA sequencing. The resulting expression vectors were named pET-28b{Ptac}[hGLYAT2(co_Ec)] (SEQ ID NO:15) and pET-28b{Ptac}[hGLYAT3(co_Ec)] (SEQ ID NO:16).

### Example 11

Production of Fatty Acid Amino Acid Adducts Via Malonyl-CoA and Acetyl-CoA by Strains Overexpressing hGLYAT2 or hGLYAT3 in a Shake Flask Experiment

The vectors produced according to Example 6 were then used to generate a microorganism strain from Table 12 below (OPX Biotechnologies Inc., USA) using any transformation method known in the art. In particular, the methods provided in section IV of WO2014026162A1 were used.

The strains generated were used for studying their ability to produce fatty acids, in particular amino acid adducts from glucose. For this purpose, the strains were transformed with the vectors pET-28b{Ptac}[hGLYAT2(co_Ec)] (SEQ ID NO:15) and pET-28b{Ptac}[hGLYAT3(co_Ec)] (SEQ ID NO:16) and cultured in shake flasks (Subsection C of section IV of WO2014026162A1). Strain BXF_031 (OPX Biotechnologies Inc., USA) harbouring the empty vector pET-28b was used as a control.

Triplicate evaluations were performed. Briefly, overnight starter cultures were made in 50 ml of Terrific Broth including the appropriate antibiotics and incubated 16-24 hours at 30° C., while shaking at 225 rpm. These cultures were used to inoculate 150 ml cultures of each strain in SM11 minimal medium to an OD₆₀₀ of 0.8 and 5% TB culture carryover as starting inoculum, and antibiotics. 1 L SM11 medium consists of: 2 ml FM10 Trace Mineral Stock, 2.26 ml 1M MgSO₄, 30 g glucose, 200 mM MOPS (pH 7.4), 1 g/L yeast extract, 1.25 ml VM1 Vitamin Mix, 0.329 g K₂HPO₄, 0.173 g KH₂PO₄, 3 g (NH₄)₂SO₄, 0.15 g citric acid (anhydrous); FM10 Trace Mineral Stock consists of: 1 ml of concentrated HCl, 4.9 g CaCl₂*2H₂O, 0.97 g FeCl₃*6H₂O, 0.04 g CoCl₂*6H₂O, 0.27 g CuCl₂*2H₂O, 0.02 g ZnCl₂, 0.024 g Na₂MoO₄*2H₂O, 0.007 g H₃BO₃, 0.036 g MnCl₂*4H₂O, Q.S. with DI water to 100 ml; VM1 Vitamin Mix Solution consists of: 5 g Thiamine, 5.4 g Pantothenic acid, 6.0 g Niacin, 0.06 g, Q.S. with DI water to 1000 ml. All ingredients for the culture mediums used in this example are provided in (Subsection A of section IV of WO2014026162A1).

Cultures were incubated for 2 hours at 30° C., while shaking at 225 rpm. After 2 hours, the cells were washed with SM11 (SM11 medium without phosphate). Cells were twice spun down (4,000 rpm, 15 min), the supernatant decanted, the pellet re-suspended in 150 ml of SM11 (SM11 medium without phosphate). The cultures were used to inoculate 3×50 ml of each strain in SM11 (no phosphate). The cultures were grown at 30° C. for approximately 2 h to an 00600 of 1.0-1.5 after 2 h cells and shifted to 37° C. and samples removed periodically for product measurement over the course of 72 hrs.

The quantification of N-lauroylglycine, N-myristoylglycine and N-palmitoylglycine and the detection of other acylamino acids in fermentation samples was performed by HPLC-ESI/MS.

TABLE 12
List of microorganism strains that were used to introduce the genes hGLYAT2 or hGLYAT3
in Examples 10 and 11. The method of production and the sequences of the strains
are provided in Table 3.2 of WO2014026162A1 (OPX Biotechnologies Inc., USA).

| Strain | | | SEQ ID |
|---|---|---|---|
| designation | Host | Plasmid | NOs. |
| BXF_0012 | BX_864 | 1)pBMT-3_ccdAB | 17 |
| BXF_0013 | BX_864 | 1)pBMT-3_ccdAB_P_{T7}-′tesA | 18 |
| BXF_0014 | BX_864 | 1)pBMT-3_ccdAB_P_{T7}-nphT7-hbd-crt-ter | 19 |
| BXF_0015 | BX_864 | 1)pBMT-3_ccdAB_P_{T7}-′tesA_PT7-nph_{T7}-hbd-crt-ter | 20 |
| BXF_0020 | BX_860 | 1)pBMT-3_ccdAB_PT7-′tesA_PT7-nphT7-hbd-crt-ter | 20 |
| BXF_0021 | BX_876 | 1)pBMT-3_ccdAB_PT7-′tesA_PT7-nphT7-hbd-crt-ter | 20 |
| BXF_0022 | BX_874 | 1)pBMT-3_ccdAB | 17 |
| BXF_0023 | BX_874 | 1)pBMT-3_ccdAB_PT7-′tesA | 18 |
| BXF_0024 | BX_874 | 1)pBMT-3_ccdAB_PT7-′tesA_PT7-nphT7-hbd-crt-ter | 20 |
| BXF_0025 | BX_875 | 1)pBMT-3_ccdAB | 17 |
| BXF_0026 | BX_875 | 1)pBMT-3_ccdAB_PT7-′tesA | 18 |
| BXF_0027 | BX_875 | 1)pBMT-3_ccdAB_PT7-′tesA_PT7-nphT7-hbd-crt-ter | 20 |
| BXF_0028 | BX_878 | 1)pBMT-3ccdAB-T7-′tesA-PT7_nphT7_hbd_crt_ter | 20 |
| BXF_0028 | BX_878 | 1)pBMT-3_ccdAB_PT7-′tesA_PT7-nphT7-hbd-crt-ter | 20 |
| BXF_0029 | BX_879 | 1)pBMT-3_ccdAB_PT7-′tesA_PT7-nphT7-hbd-crt-ter | 20 |
| BXF_0030 | BX_881 | 1)pBMT-3_ccdAB_PT7-′tesA_PT7-nphT7-hbd-crt-ter | 20 |
| BXF_0031 | BX_864 | 1)pBMT-3_ccdAB_PT7-′tesA_PT7-nphT7-hbd-crt-ter | 20 |
| | | 2)pET-28b(empty vector) | 21 |
| BXF_0033 | BX_878 | 1)pBMT-3_ccdAB_PT7-nphT7-hbd-crt-ter | 19 |
| BXF_0034 | BX_879 | 2)pBMT-3_ccdAB_PT7-nphT7-hbd-crt-ter | 19 |

### Example 12

Generation of a Vector for Deletion of the gcvTHP Operon in Escherichia coli W3110 ΔfadE

To generate a vector for the deletion of the gcvTHP operon of E. coli W3110, which encodes a glycine cleavage system (GcvT: aminomethyltransferase,tetrahydrofolate-dependent, subunit (T protein) of glycine cleavage complex; GcvH: glycine cleavage complex lipoylprotein; GcvP: glycine decarboxylase, PLP-dependent, subunit (protein P) of glycine cleavage complex), approx. 500 bp upstream and downstream of the GcvTHP operon were amplified via PCR. The upstream region of GcvTHP was amplified using the oligonucleotides o-MO-40 (SEQ ID NO:22) and o-MO-41 (SEQ ID NO:23) The downstream region of gcvTHP was amplified using the oligonucleotides o-MO-42 (SEQ ID NO:24) and o-MO-43 (SEQ ID NO:25). The PCR procedure is described above in Example 3.

In each case PCR fragments of the expected size could be amplified (PCR 1,553 bp, (SEQ ID NO:26); PCR 2,547 bp, SEQ ID NO:27). The PCR samples were separated via agarose gel electrophoresis and DNA fragments were isolated with QiaQuick Gel extraction Kit (Qiagen, Hilden). The purified PCR fragments were cloned into the vector pKO3 (SEQ ID NO:28), and cut with BamHI using the Geneart® Seamless Cloning and Assembly Kit (Life Technologies, Carlsbad, Calif., USA). The assembled product was transformed into chemically competent E. coli DH5α cells (New England Biolabs, Frankfurt). Procedure of PCR purification, in-vitro cloning and transformation were carried out according to manufacturer's manual. The correct insertion of the target genes was checked by restriction analysis and the authenticity of the introduced DNA fragments was verified by DNA sequencing. The resulting knock-out vector was named pKO3 delta gcvTHP (SEQ ID NO:29).

The construction of strain E. coli W3110 ΔfadE ΔgcvTHP was carried out with the help of pKO3 delta gcvTHP using the method described in Link et al., 1997. The DNA sequence after deletion of gcvTHP is SEQ ID NO:30. The E. coli strain W3110 ΔfadEΔgcvTHP was transformed with the plasmid pCDF{Ptac}[hGLYAT2(co_Ec)-fadD_Ec]{Placuv5}[alkLmod1] (SEQ ID NO:13, Example 3) by means of electroporation and plated onto LB-agar plates supplemented with spectinomycin (100 μg/mL). Transformants were checked for the presence of the correct plasmids by plasmid preparation and analytic restriction analysis. The resulting strain was named E. coli W3110 ΔfadEΔΔgcvTHP pCDF{Ptac}[hGLYAT2(co_Ec)-fadD_Ec]{Placuv5}[alkLmod1].

### Example 13

Production of Lauroylglycinate by E. coli Strains with Deletion in the fadE or fadE/gcvTHP Gene, Overexpressing the hGLYAT2, fadD and alkL Genes

The strain generated in Example 1 was used to study its ability to produce more lauroylglycinate, in comparison to the reference strain without gcvTHP deletion.

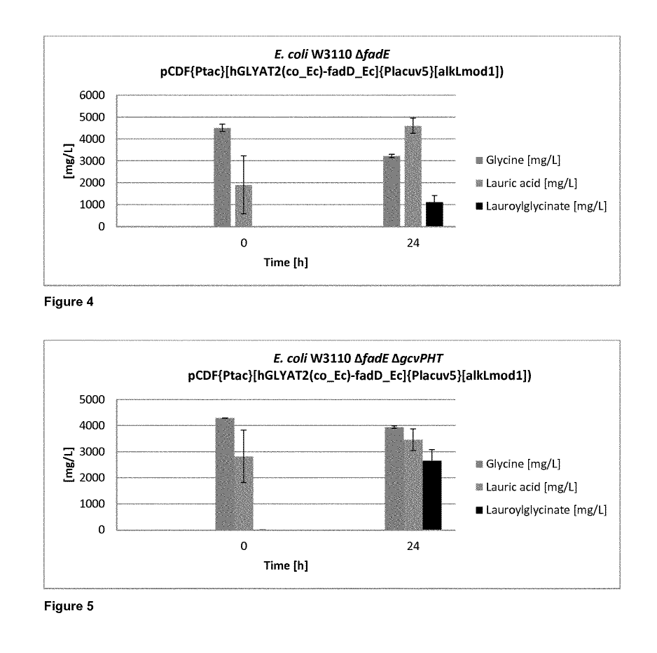

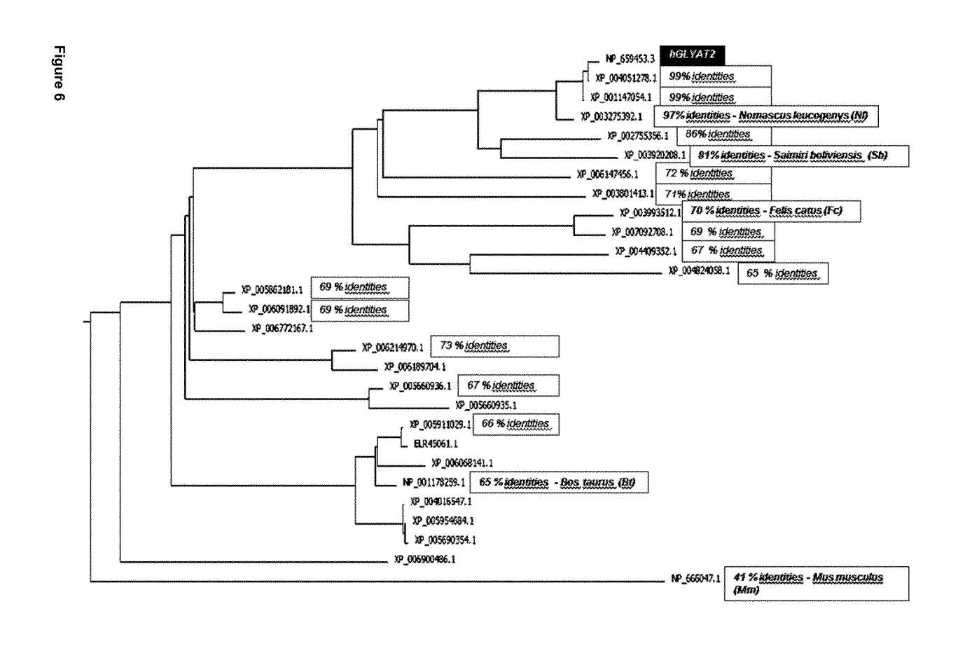

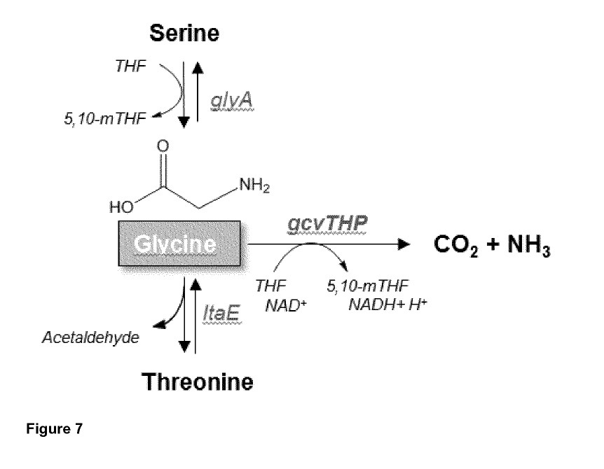

Starting from a −80° C. glycerol culture, the strains to be studied were first plated onto an LB-agar plate supplemented with 100 μg/mL spectinomycin and incubated overnight at 37° C. Starting from a single colony in each case, the strains were then grown as a 5-mL preculture in LB-broth, Miller (Merck, Darmstadt) supplemented with 100 μg/mL spectinomycin. The further culture steps were performed in M9-FIT medium. The medium, composed of 38 mM disodium hydrogenphosphate dihydrate, 22 mM potassium dihydrogenphosphate, 8.6 mM sodium chloride, 37 mM ammonium chloride, 2 mM magnesium sulphate heptahydrate (all chemicals from Merck, Darmstadt), 5% (w/v) maltodextrin solution (dextrose equivalent 13.0-17.0, Sigma Aldrich, Taufkirchen), 1% (w/v) amyloglycosidase from Aspergillus niger (Sigma-Aldrich, Taufkirchen), 1 drop Delamex 180 (Bussetti & Co, Wien) and 0.1% (v/v) trace element solution, was brought to pH 7.4 with 25% strength ammonium hydroxide solution. The trace element solution added, composed of 9.7 mM manganese(II) chloride tetrahydrate, 6.5 mM zinc sulphate heptahydrate, 2.5 mM sodium-EDTA (Titriplex III), 4.9 mM boric acid, 1 mM sodium molybdate dihydrate, 32 mM calcium chloride dihydrate, 64 mM iron(II) sulphate heptahydrate and 0.9 mM copper(II) chloride dihydrate, dissolved in 1 M hydrochloric acid (all chemicals from Merck, Darmstadt) was filter-sterilized before being added to the M9 medium. 20 mL of M9 medium supplemented with 100 μg/mL spectinomycin were introduced into baffled 100-mL Erlenmeyer flasks and inoculated with 0.5 mL preculture. The flasks were cultured at 37° C. and 200 rpm in a shaker-incubator. After a culture time of 8 hours, 50 mL of M9 medium supplemented with 100 μg/mL spectinomycin and were introduced into a baffled 250-mL Erlenmeyer flask and inoculated with the 10-mL culture to achieve an optical density (600 nm) of 0.1. The flasks were cultured at 37° C. and 200 rpm in a shaker-incubator. When an optical density (600 nm) of 0.6 to 0.8 was reached, gene expression was induced by addition of 1 mM IPTG. The strains were cultured for a further 48 hours at 37° C. and 200 rpm. 1-3 h after the induction, 6 g/L glycine and 6 g/L lauric acid (dissolved in lauric acid methyl ester) were added to the cultures. After 0 h and 24 h cultivation samples were taken, and lauroylglycinate, lauric acid and glycine present were analysed. The results are shown in FIGS. 4 and 5. It has been possible to demonstrate that both E. coli strains W3110 ΔfadE pCDF{Ptac}[hGLYAT2(co_Ec)-fadD_Ec]{Placuv5}[alkLmod1] and E. coli strain W3110 ΔfadE ΔfadE pCDF{Ptac}[hGLYAT2(co_Ec)-fadD_Ec]{Placuv5}[alkLmod1] are capable of forming lauroylglycinate. But in the new strain background ΔfadE ΔgcvTHP higher amounts of lauroylglycinate were synthesized and higher amounts of glycine are detected. It appears that less glycine is metabolized in the ΔgcvTHP background and can be used for lauroylglycinate synthesis.

### Example 14

Generation of a Vector for Deletion of the glyA Gene in Escherichia coli W3110 ΔfadE

To generate a vector for the deletion of the glyA-gene encoding a component of the Glycine hydroxymethyltransferase of E. coli W3110 approx. 500 bp upstream and downstream of the glyA-gene were amplified via PCR. The upstream region of glyA was amplified using the oligonucleotides o-MO-44 (SEQ ID NO:31) and o-MO-45 (SEQ ID NO:32). The downstream region of glyA was amplified using the oligonucleotides o-MO-46 (SEQ ID NO:33) and o-MO-47 (SEQ ID NO:34).

In each case PCR fragments of the expected size could be amplified (PCR 1,546 bp, (SEQ ID NO:35); PCR 2,520 bp, SEQ ID NO:36). The PCR samples were separated via agarose gel electrophoresis and DNA fragments were isolated with QiaQuick Gel extraction Kit (Qiagen, Hilden). The purified PCR fragments were assembled via a crossover PCR. The generated fragment was purified and subcloned into the cloning vector pCR®-Blunt IITOPO (Life technologies) according to manufacturer's manual. To clone the fragment into the target plasmid pKO3 (SEQ ID NO:28) it was amplified with flanking BamHI restriction sites, using the oligonucleotides o-MO-52 (SEQ ID NO:37) and o-MO-53 (SEQ ID NO:38). The purified, BamHI cleaved PCR 3 fragment (SEQ ID NO:39) was ligated into the correspondingly cleaved vector pKO3 (SEQ ID N0:28). The assembled product was transformed into chemically competent E. coli DH5α cells (New England Biolabs, Frankfurt). Procedure of PCR purification, in-vitro cloning and transformation were carried out according to manufacturer's manual. The correct insertion of the target genes was checked by restriction analysis and the authenticity of the introduced genes was verified by DNA sequencing. The resulting knock-out vector was named pKO3 delta glyA (SEQ ID NO:40).

The construction of strain E. coli W3110 ΔfadE ΔglyA was carried out with the help of pKO3 delta GlyA using the method described in Link et al., 1997. SEQ ID NO:41 is the DNA sequence after deletion of glyA. The E. coli strain W3110 ΔfadE ΔgcvTHP was transformed with the plasmid pCDF{Ptac}[hGLYAT2(co_Ec)-fadD_Ec]{Placuv5}[alkLmod1] (SEQ ID NO:13 from Example 3), by means of electroporation and plated onto LB-agar plates supplemented with spectinomycin (100 μg/mL). Transformants were checked for the presence of the correct plasmids by plasmid preparation and analytic restriction analysis. The resulting strain was named E. coli W3110 ΔfadE ΔglyA pCDF{Ptac}[hGLYAT2(co_Ec)-fadD_Ec]{Placuv5}[alkLmod1].

### Example 15

Generation of a Vector for Deletion of the taE Gene in Escherichia coli W3110 ΔfadE

To generate a vector for the deletion of the ItaE-gene encoding the L-allo-threonine aldolase of E. coli W3110 approximately 500 bp upstream and downstream of the ItaE were amplified via PCR as described above. The upstream region of ItaE was amplified using the oligonucleotides ItaE-UP_fw (SEQ ID NO:42) and ItaE-UP-XhoI_rev (SEQ ID NO:43). The downstream region of ItaE was amplified using the oligonucleotides ItaE-DOWN_fw (SEQ ID NO:44) and ItaE-DOWN_rev (SEQ ID NO:45).

In each case PCR fragments of the expected size could be amplified (PCR 4,550 bp, (SEQ ID NO:46); PCR 5, 536 bp, SEQ ID NO:47). The PCR samples were separated via agarose gel electrophoresis and DNA fragments were isolated with QiaQuick Gel extraction Kit (Qiagen, Hilden). The purified PCR fragments were assembled via a crossover PCR. The generated fragment was purified and cloned into the cloning vector pCR®-Blunt IITOPO (Life technologies) according to manufacturer's manual. To clone the fragment into the target plasmid pKO3 (SEQ ID NO:28) it was amplified with flanking BamHI restriction sites, using the oligonucleotides o-MO-54 (SEQ ID NO:48) and o-MO-55 (SEQ ID NO:49). The purified, BamHI cleaved PCR 6 fragment (SEQ ID NO:50) was ligated into the correspondingly cleaved vector pKO3 (SEQ ID N0:28). The assembled product was transformed into chemically competent E. coli DH5α cells (New England Biolabs, Frankfurt). Procedure of PCR purification, in-vitro cloning and transformation were carried out according to manufacturer's manual. The correct insertion of the target genes was checked by restriction analysis and the authenticity of the introduced genes was verified by DNA sequencing. The resulting knock-out vector was named pKO3 delta ItaE (SEQ ID NO:51).

The construction of strain E. coli W3110 ΔfadE ΔItaE was carried out with the help of pKO3 delta ItaE using the method described in Link et al., 1997. The DNA sequence after deletion of ItaE is described in SEQ ID NO:52). The E. coli strain W3110 ΔfadE ΔItaE was transformed with the plasmid pCDF{Ptac}[hGLYAT2(co_Ec)-fadD_Ec]{Placuv5}[alkLmod1] (SEQ ID NO:13 from Example 3), by means of electroporation and plated onto LB-agar plates supplemented with spectinomycin (100 μg/mL). Transformants were checked for the presence of the correct plasmids by plasmid preparation and analytic restriction analysis. The resulting strain was named E. coli W3110 ΔfadEΔItaE pCDF{Ptac}[hGLYAT2(co_Ec)-fadD_Ec]{Placuv5}[alkLmod1].

### Example 16

### LC-ESI/MS²-Based Quantification of Lauric Acid

Quantification of lauric acid in fermentation samples was carried out by means of LC-ESI/MS² on the basis of an external calibration for lauric acid (0.1-50 mg/L) and by using the internal standard d3-LS.

The following instruments were used:
HPLC system 1260 (Agilent; Böblingen) with Autosampler (G1367E), binary pump (G1312B) and thermo-statted column (G1316A)
Mass spectrometer TripelQuad 6410 (Agilent; Böblingen) with ESI source
HPLC column: Kinetex C18, 100×2.1 mm, particle size: 2.6 μm, pore size 100 Å (Phenomenex; Aschaffenburg)
Pre-column: KrudKatcher Ultra HPLC In-Line Filter; 0.5 μm filter depth and 0.004 mm inner diameter (Phenomenex; Aschaffenburg)

The samples were prepared by pipetting 1900 μL of solvent (80% (v/v) ACN, 20% double-distilled H₂O (v/v), +0.1% formic acid) and 100 μL of sample into a 2 mL reaction vessel. The mixture was vortexed for approx. 10 seconds and then centrifuged at approx. 13 000 rpm for 5 min. The clear supernatant was removed using a pipette and analysed after appropriate dilution with a diluent (80% (v/v) ACN, 20% double-distilled H₂O (v/v), +0.1% formic acid). In each case, 100 μL of ISTD were added to 900 μl of sample (10 μl with a sample volume of 90 μl).

HPLC separation was carried out using the above-mentioned column and pre-column. The injection volume is 1.0 μl, the column temperature 50° C., the flow rate is 0.6 ml/min. the mobile phase consists of eluent A (0.1% strength (v/v) aqueous formic acid) and eluent B (acetonitrile with 0.1% (v/v) formic acid). The gradient shown it Table 4 was utilized:

TABLE 13
Concentrations of Eluent A and B used in Example 12

| Time | Eluent A | Eluent B |
|---|---|---|
| [min] | [%] | [%] |
| 0 | 85 | 15 |
| 1 | 85 | 15 |
| 5 | 2 | 98 |
| 8 | 2 | 98 |
| 8.1 | 85 | 15 |
| 12 | 85 | 15 |

ESI-MS² analysis was carried out in positive mode with the following parameters of the ESI source:
Gas temperature 320° C.
Gas flow 11 L/min
Nebulizer pressure 50 psi
Capillary voltage 4000 V

Detection and quantification of lauric acid was carried out with the following MRM parameters.

TABLE 14
MRM parameters used in detection and quantification of lauric acid

| | Precursor ion | Product ion | Collision energy |
|---|---|---|---|
| Analyte | [m/z] | [m/z] | [eV] |
| LS | 201.1 | 201.1 | 110 |
| d3-LS | 204.1 | 204.1 | 110 |

### Example 17

### Detection of Glycine

Detection of glycine was performed via derivatization with ortho-phthaldialdehyde (OPA) and UV/VIS detection using an Agilent 1200 HPLC system.

200 μL of a homogeneous fermentation broth simple was mixed with 1800 μL of 30% (v/v) 1-propanol, vortexed for 10 s and subsequently centrifuged at 13,000×g for 5 min. The supernatant was removed and used for HPLC analysis using the following parameters:

| Mobile phase: | Eluent A | | | |
|---|---|---|---|---|
| | 2.5 mL acetic acid per 1 L distilled water, pH | | | |
| | adjustment with NaOH @ 6.0 | | | |
| | Eluent B | | | |
| | Methanol | | | |
| Column: | Luna 5μ C8 100 A (100 × 4.6 mm); | | | |
| | Phenomenex | | | |
| Column oven | 40° C. | | | |
| temperature: | | | | |
| Flow: | 1.0 mL/min | | | |
| Gradient: | Time | % B | Flow | Max. Press. |
| | 0.0 | 30.0 | 1.0 | 400 |
| | 1.0 | 30.0 | 1.0 | 400 |
| | 17.0 | 90.0 | 1.0 | 400 |
| | 19.5 | 90.0 | 1.0 | 400 |
| | 19.6 | 30.0 | 1.0 | 400 |
| | 20.5 | 30.0 | 1.0 | 400 |

| Run time: | 22 min |
|---|---|
| Detector: | DAD |
| | 334 nm |
| | Spectrum |
| | Store: all |
| | Range: 200-400 nm |
| | step 2 nm |
| | FLD (excitation @ 330 nm; emission @ 450 nm, |
| | PMT gain 13) |
| Derivatization: | automatically with injection program: |
| | Inject Programm |

| | |
|---|---|
| # | Command |
| 1 | DRAW 4.5 μL from Vial 1*, def. |
| | speed, def. offset |
| 2 | DRAW 1.5 μL from sample, def. |
| | speed, def. offset |
| 3 | DRAW 0.5 μL from air, def. speed |
| 4 | NEEDLE wash in flush Port. 15.0 sec |
| 5 | DRAW 4.5 μL from Vial 1, def. |
| | speed, def. offset |
| 6 | MIX 11.0 μL in seat, def. |
| | speed, 1 times |
| 7 | WAIT 1.00 min |
| 8 | INJECT |
| 9 | WAIT 0.50 min |
| 10 | VALVE bypass |
| 11 | Draw 100.0 μL from Vial 2*, |
| | def. speed, def. offset |
| 12 | Eject 100.0 μL from Vial 2, |
| | def. speed, def. offset |
| 13 | Valve mainpass |

*vial 1 contains OPA reagent (see below); vial 2 contains water

### Preparation of OPA Reagent

100 mg o-phthaldialdehyde was dissolved in 1 ml methanol and subsequently 0.4 mM borate buffer (pH 10.4) was added to give 10 mL. Subsequently, 50 μL mercaptoethanol was added and the reagent stored at 4° C. Additional 10 μL mercaptoethanol was added before use.

Preparation of Borate Buffer (0.4 mM H₃BO₄):

38.1 g Na₂B₄O₇*10 H₂O (0.1 mol) were dissolved in 1 L distilled water and the pH adjusted to 10.4 M NaOH auf 10.4 eingestellt. Subsequently, 1 mL 25% Brij35 (v/v) was added.

| | |
|---|---|
| Retention time: Glycine: | 7.153 min |

### Example 18

Generation of Vectors for the Expression of hGLYAT2-Homologs

To generate vectors for the expression of N-acyltransferases of different organisms, variants found in the NCBI databases with homology to HGLYAT2 were synthesized and codon-optimized for E. coli. These were glycine N-acyltransferase-like protein 2 isoform 1 of Nomascus leucogenys (NI, XP_003275392.1, SEQ ID NO:53), glycine N-acyltransferase-like protein 2 of Saimiri boliviensis (Sb, XP_003920208.1, SEQ ID NO:54), glycine-N-acyltransferase-like 2 of Felis catus (Fc, XP_003993512.1, SEQ ID NO:55), glycine N-acyltransferase-like protein 2 of Bos taurus (Bt, NP_001178259.1, SEQ ID NO:56), and glycine N-acyltransferase of Mus musculus (Mm, NP_666047.1, SEQ ID NO:57).

The hGLYAT2-gene of pCDF{Ptac}[hGLYAT2(co_Ec)-fadD_Ec]{Placuv5}[alkLmod1] (SEQ ID NO:13 of Example 3) was replaced by this variants as follows: The synthesized DNA fragments were digested with the restriction endonucleases BamHI and AsiSI and ligated into the correspondingly cut pCDF{Ptac}[hGLYAT2(co_Ec)-fadD_Ec]{Placuv5}[alkLmod1].

The correct insertion of the target genes was checked by restriction analysis and the authenticity of the introduced genes was verified by DNA sequencing. The resulting expression vectors were named:
pCDF{Ptac}[GLYAT_NI(co_Ec)-fadD_Ec]{Placuv5}[alkLmod1]
pCDF{Ptac}[GLYAT_Sb(co_Ec)-fadD_Ec]{Placuv5}[alkLmod1]
pCDF{Ptac}[GLYAT_Fc(co_Ec)-fadD_Ec]{Placuv5}[alkLmod1]
pCDF{Ptac}[GLYAT_Bt(co_Ec)-fadD_Ec]{Placuv5}[alkLmod1]
pCDF{Ptac}[GLYAT_Mm(co_Ec)-fadD_Ec]{Placuv5}[alkLmod1]

### Example 19

Production of Lauroylglycinate by E. coli Strains with Deletion in the fadE Gene, Overexpressing the hGLYAT-Variants, fadD and alkL Genes

The strains generated in Example 18 were used to study their ability to produce lauroylglycinate, in comparison to the reference strain expressing hGLYAT2 harbouring the plasmid pCDF{Ptac}[hGLYAT2(co_Ec)-fadD_Ec]{Placuv5}[alkLmod1], applying the protocol described in Example 13.

1-3 h after the induction, 6 g/L glycine and 6 g/L lauric acid (dissolved in lauric acid methyl ester) were added to the cultures. After cultivation time of 48 h the entire broth of a shake flask was extracted with Acetone (ratio 1:2). Further sample treatment is described in Example 7. Samples were taken, and lauroylglycinate, lauric acid and glycine present were analysed. The results are shown in Table 15.

All strains except the none-plasmid control produced lauroylglycinate in amounts between 0.44 and 2109.8 mg/L.

TABLE 15
Quantitative determination of lauroylglycinate after a cultivation time
of 48 h in strains of E. coli W3110 ΔfadE harboring different plasmids.
Each strain was fed with 6 g/L glycine and 6 g/L lauric acid

| | | C_{lauroylglycinate} |
|---|---|---|
| strain | plasmid | [mg/L] |
| E. coli | — | 0.0 |
| W3110 | pCDF{Ptac}[hGLYAT2(co_Ec)-fadD_Ec]{Placuv5}[alkLmod1] | 2109.8 |
| ΔfadE | pCDF{Ptac}[GLYAT_Fc(co_Ec)-fadD_Ec]{Placuv5}[alkLmod1] | 1.1 |
| | pCDF{Ptac}[GLYAT_Sb(co_Ec)-fadD_Ec]{Placuv5}[alkLmod1] | 1.5 |
| | pCDF{Ptac}[GLYAT_Mm(co_Ec)-fadD_Ec]{Placuv5}[alkLmod1] | 0.5 |
| | pCDF{Ptac}[GLYAT_Nl(co_Ec)-fadD_Ec]{Placuv5}[alkLmod1] | 209.7 |
| | pCDF{Ptac}[GLYAT_Bt(co_Ec)-fadD_Ec]{Placuv5}[alkLmod1] | 0.4 |

### Example 20

### Construction of Mutants Defect in Different Glycine-Metabolizing Pathways

With the Knock out-plasmid described in Examples 12, 14 and 15, different mutants were constructed. The construction of each strain was performed with the help of the plasmids pKO3 delta ItaE (SEQ ID NO:51), pKO3 delta GlyA (SEQ ID NO: 40) and pKO3 delta gcvTHP (SEQ ID NO:29) using strain E. coli W3110 ΔfadE with the method described in Link et al., 1997. The E. coli strains W3110 ΔfadE ΔgcvTHP ΔItaE, W3110 ΔfadE ΔgcvTHP ΔglyA, W3110 ΔfadE ΔglyA ΔItaE and W3110 ΔfadE ΔgcvTHP ΔItaE ΔglyA were each transformed with the plasmid pCDF{Ptac}[hGLYAT2(co_Ec)-fadD_Ec]{Placuv5}[alkLmod1] (SEQ ID NO:13 from Example 3), by means of electroporation and plated onto LB-agar plates supplemented with spectinomycin (100 μg/mL). Transformants were checked for the presence of the correct plasmids by plasmid preparation and analytic restriction analysis. The resulting strains were named E. coli W3110 ΔfadE ΔgcvTHP ΔItaE pCDF{Ptac}[hGLYAT2(co_Ec)-fadD_Ec]{Placuv5}[alkLmod1], E. coli W3110 ΔfadE ΔgcvTHP ΔglyA pCDF{Ptac}[hGLYAT2(co_Ec)-fadD_Ec]{Placuv5}[alkLmod1], E. coli W3110 ΔfadE ΔglyA ΔItaE pCDF{Ptac}[hGLYAT2(co_Ec)-fadD_Ec]{Placuv5}[alkLmod1] and W3110 ΔfadE ΔgcvTHP ΔItaE ΔglyA pCDF{Ptac}[hGLYAT2(co_Ec)-fadD_Ec]{Placuv5}[alkLmod1].

### Example 21

Generation of a Vector for Expression of the Porin-Gene fadL Instead of alkL in Escherichia coli W3110 ΔfadE Overexpressing the hGLYAT2 and fadD

To generate a vector for the expression of fadL instead of alkL, the fadL gene was amplified from chromosomal DNA of E. coli W3110 by means of sequence-specific oligonucleotides fadL_ec-fp and fadL_EC_rp (SEQ IDs No. 66 and 67). The promoter region (Placuv5) was amplified from the target vector pCDF{Ptac}[hGLYAT2(co_Ec)-fadD_Ec]{Placuv5}[alkLmod1] (SEQ ID NO:13) using oligonucleotides upstream_fp and upstream_rp (SEQ IDs No. 68 and 69). The fragments were fused using PCR and cloned to target vector opened NsiI/BamHI using the Geneart® Seamless Cloning and Assembly Kit (Life Technologies, Carlsbad, Calif., USA). The assembled product was transformed into chemically competent E. coli DH5α cells (New England Biolabs, Frankfurt).

The correct insertion of the target genes was checked by restriction analysis and the authenticity of the introduced genes was verified by DNA sequencing. The resulting expression vector was named pCDF{Ptac}[hGLYAT2(co_Ec)-fadD_Ec]{Placuv5}[fadL] (SEQ ID NO:70).

The E. coli strain W3110 ΔfadE ΔgcvTHP generated in Example 12 was transformed with the plasmid pCDF{Ptac}[hGLYAT2(co_Ec)-fadD_Ec]{Placuv5}[fadL] by means of electroporation and plated onto LB agar plates supplemented with spectinomycin (100 μg/mL). Transformants were checked for the presence of the correct plasmid by plasmid preparation and analytic restriction analysis. The resulting strain was named E. coli W3110 ΔfadE ΔgcvTHP pCDF{Ptac}[hGLYAT2(co_Ec)-fadD_Ec]{Placuv5}[fadL].

### Example 22

Generation of a Vector for Deletion of the Kbl Gene in Escherichia coli W3110 ΔfadE

To generate a vector for the deletion of the kbl-gene encoding the 2-Amino-3-Ketobutyrate CoA-Ligase of E. coli W3110 approx. 500 bp upstream and downstream of the kbl-gene were amplified via PCR. The upstream region of kbl was amplified using the oligonucleotides 1960_up_fp (SEQ ID NO:71) and 1960_up_rp (SEQ ID NO:72). The downstream region of kbl was amplified using the oligonucleotides 1960_down_fp (SEQ ID NO:73) and 1960_down_rp (SEQ ID NO:74).

In each case PCR fragments of the expected size could be amplified. The PCR samples were separated via agarose gel electrophoresis and DNA fragments were isolated with QiaQuick Gel extraction Kit (Qiagen, Hilden). The purified PCR fragments were assembled via a crossover PCR. The generated fragment was purified, BsaI and SalI cleaved and ligated into the correspondingly cleaved vector pKO3 (SEQ ID NO:28). The assembled product was transformed into chemically competent E. coli DH5α cells (New England Biolabs, Frankfurt). Procedure of PCR purification, in-vitro cloning and transformation were carried out according to manufacturer's manual. The correct insertion of the target genes was checked by restriction analysis and the authenticity of the introduced genes was verified by DNA sequencing. The resulting knock-out vector was named pKO3 delta kbl (SEQ ID NO:75).

The construction of strain E. coli W3110 ΔfadE Δkbl was carried out with the help of pKO3 delta kbl using the method described in Link et al., 1997. SEQ ID NO:76 is the DNA sequence after deletion of kbl. The E. coli strain W3110 ΔfadE ΔgcvTHP was transformed with the plasmid pCDF{Ptac}[hGLYAT2(co_Ec)-fadD_Ec]{Placuv5}[alkLmod1] (SEQ ID NO:13 from Example 3), by means of electroporation and plated onto LB-agar plates supplemented with spectinomycin (100 μg/mL). Transformants were checked for the presence of the correct plasmids by plasmid preparation and analytic restriction analysis. The resulting strain was named E. coli W3110 ΔfadE Δkbl pCDF{Ptac}[hGLYAT2(co_Ec)-fadD_Ec]{Placuv5}[alkLmod1].

### Example 23

Generation of Strains which Overexpress the Escherichia coli fadD and Homo sapiens hGLYAT2 Under Control of the Ptrc Promoter Instead of Ptac

To generate E. coli strains which overexpress the Escherichia coli fadD gene in combination with the Homo sapiens hGLYAT2 under control of the Ptrc promoter (Brosius et al. 1985), the promoter region of plasmid pCDF{Ptac}[hGLYAT2(co_Ec)-fadD_Ec]{Placuv5}[alkLmod1] was changed to the sequence of the trc-promoter by PCR and usage of sequence-specific oligonucleotides. The new plasmid was named pCDF{Ptrc}[hGLYAT2(co_Ec)-fadD_Ec]{Placuv5}[alkLmod1] SEQ ID NO:77

Analogous to the description in Examples 12 and 20, the following E. coli W3110 strains were constructed:
ΔfadE ΔgcvTHP pCDF{Ptrc}[hGLYAT2(co_Ec)-fadD_Ec]{Placuv5}[alkLmod1],
ΔfadEΔgcvTHP ΔItaE pCDF{Ptrc}[hGLYAT2(co_Ec)-fadD_Ec]{Placuv5}[alkLmod1],
ΔfadE ΔglyA ΔItaE pCDF{Ptrc}[hGLYAT2(co_Ec)-fadD_Ec]{Placuv5}[alkLmod1],
ΔfadE ΔgcvTHP Δkbl pCDF{Ptrc}[hGLYAT2(co_Ec)-fadD_Ec]{Placuv5}[alkLmod1],
ΔfadE ΔgcvTHP ΔItaE Δkbl pCDF{Ptrc}[hGLYAT2(co_Ec)-fadD_Ec]{Placuv5}[alkLmod1] and
ΔfadE ΔgcvTHP ΔItaE ΔglyA pCDF{Ptrc}[hGLYAT2(co_Ec)-fadD_Ec]{Placuv5}[alkLmod1].

### Example 24

Production of Acyl Glycinate by E. coli Strains from Hydrolysed Coconut Oil and Glycine

The E. coli strain W3110 ΔfadE pCDF{Ptac}[hGLYAT2(co_Ec)-fadD_Ec]{Placuv5}[alkLmod1] generated as described in Example 5 was fermented in a fed-batch fermentation to study the ability of linking fatty acids from hydrolysed coconut oil and glycine to give acyl amino acids, e.g. lauroylglycinate. This fermentation was carried out in a parallel fermentation system from DASGIP with 8 bioreactors.

The experimental setting was as described in Example 8 except for the following modifications: Fatty acids from hydrolysed coconut oil are solid at 30° C. and cannot be used as a fluidic feed in a microbial fermentation process. To overcome this problem the temperature shift from 37° C. to 30° C. before induction of the heterologous genes was omitted. The entire process was run at 37° C. from start to finish. Induction was triggered 2 h after feedstart.

Biotransformation was started 7 h after induction by adding 10 g fatty acids from hydrolysed coconut oil and 100 mL of a 100 g/L aqueous solution of glycine, yielding 24 g/L of each substrate referring to the whole fermentation broth.

To monitor the conversion samples were taken after 47 h and 65 h of biotransformation and analysed as described in Example 7. The results are shown in Table 16.

TABLE 16
Concentrations of different Acyl glycinates, free fatty acids and glycine after 47 h and 65 h of biotransformation.

| Fermentation | | | | | | |
|---|---|---|---|---|---|---|
| time [h] | C_{Lauroyl glycinate} [g/l] | C_{Myristoyl glycinate} [g/l] | C_{Palmitoyl glycinate} [g/l] | C_{Glycine} [g/l] | C_{Octanoic acid} [g/l] | C_{Decanoic acid} [g/l] |
| 47 | 7.4 | 1.1 | 0.4 | 0.0 | 0.1 | 0.01 |
| 65 | 5.7 | 0.8 | 0.4 | 0.0 | 0.0 | 0.0 |

| Fermentation | | | | | | |
|---|---|---|---|---|---|---|
| time [h] | C_{Lauric acid} [g/l] | C_{Myristic acid} [g/l] | C_{Palmitoleic acid} [g/l] | C_{Palmitic acid} [g/l] | C_{Oleic acid} [g/l] | C_{Stearic acid} [g/l] |
| 47 | 1.0 | 1.0 | 0.0 | 0.5 | 0.8 | n.d. |
| 65 | 0.8 | 0.7 | 0.0 | 0.3 | 0.5 | n.d. |

It was shown, that E. coli W3110 ΔfadE pCDF{Ptac}[hGLYAT2(co_Ec)-fadD_Ec]{Placuv5}[alkLmod1] is able to link not only lauric acid but also fatty acids of other chain lengths from hydrolysed coconut oil to glycine forming Acyl glycinates.

### Example 25

Production of Acyl Glycinate by E. coli Strains with Deletions in Glycine Metabolizing Pathways from Hydrolysed Coconut Oil and Glycine

The E. coli strains as generated in Examples 12 and 23, E. coli W3110
ΔfadE ΔgcvTHP pCDF{Ptac}[hGLYAT2(co_Ec)/fadD_Ec] {Plavuv5}[alkLmod1]
ΔfadE ΔgcvTHP pCDF{Ptrc}[hGLYAT2(co_Ec)-fadD_Ec]{Placuv5}[alkLmod1]
ΔfadE ΔgcvTHP ΔItaE pCDF{Ptrc}[hGLYAT2(co_Ec)-fadD_Ec]{Placuv5}[alkLmod1]
ΔfadE ΔglyA pCDF{Ptrc}[hGLYAT2(co_Ec)-fadD_Ec]{Placuv5}[alkLmod1]
   were cultivated and biotransformation carried out as described in Example 24 and compared to the results of Example 24. To monitor the conversion samples were taken after 47 h and 65 h of biotransformation and analysed as described in Example 7. The results are shown in Table 17 to 20.

TABLE 17
Concentrations of different Acyl glycinates, free fatty acids and glycine after 47 h and 65 h of
biotransformation using E. coli W3110 ΔfadE ΔgcvTHP pCDF{Ptac} [hGLYAT2(co_Ec)-
fadD_Ec]{Placuv5}[alkLmod1] as biocatalyst.

| Fermentation | | | | | | |
|---|---|---|---|---|---|---|
| time [h] | C_{Lauroyl glycinate} [g/l] | C_{Myristoyl glycinate} [g/l] | C_{Palmitoyl glycinate} [g/l] | C_{Glycine} [g/l] | C_{Octanoic acid} [g/l] | C_{Decanoic acid} [g/l] |
| 47 | 9.3 | 2.4 | 0.9 | 9.7 | 0.0 | 0.0 |
| 65 | 6.6 | 1.7 | 0.6 | 7.0 | 0.0 | 0.0 |

| Fermentation | | | | | | |
|---|---|---|---|---|---|---|
| time [h] | C_{Lauric acid} [g/l] | C_{Myristic acid} [g/l] | C_{Palmitoleic acid} [g/l] | C_{Palmitic acid} [g/l] | C_{Oleic acid} [g/l] | C_{Stearic acid} [g/l] |
| 47 | 0.0 | 0.1 | 0.0 | 0.2 | 0.9 | n.d. |
| 65 | 0.0 | 0.0 | 0.0 | 0.0 | 0.3 | n.d. |

TABLE 18
Concentrations of different Acyl glycinates, free fatty acids and glycine after 47 h and 65 h of
biotransformation using E. coli W3110 ΔfadE ΔgcvTHP pCDF{Ptrc} [hGLYAT2(co_Ec)-
fadD_Ec]{Placuv5}[alkLmod1] as biocatalyst.

| Fermentation | | | | | | |
|---|---|---|---|---|---|---|
| time [h] | C_{Lauroyl glycinate} [g/l] | C_{Myristoyl glycinate} [g/l] | C_{Palmitoyl glycinate} [g/l] | C_{Glycine} [g/l] | C_{Octanoic acid} [g/l] | C_{Decanoic acid} [g/l] |
| 47 | 10 | 2.8 | 0.9 | 10.5 | 0.0 | 0.0 |
| 65 | 7.5 | 2.1 | 0.7 | 8.4 | 0.0 | 0.0 |

| Fermentation | | | | | | |
|---|---|---|---|---|---|---|
| time [h] | C_{Lauric acid} [g/l] | C_{Myristic acid} [g/l] | C_{Palmitoleic acid} [g/l] | C_{Palmitic acid} [g/l] | C_{Oleic acid} [g/l] | C_{Stearic acid} [g/l] |
| 47 | 0.0 | 0.0 | 0.0 | 0.1 | 0.5 | n.d. |
| 65 | 0.0 | 0.0 | 0.0 | 0.0 | 0.2 | n.d. |

TABLE 19
Concentrations of different Acyl glycinates, free fatty acids and glycine after 47 h and 65 h of
biotransformation using E. coli W3110 ΔfadE ΔgcvTHP ΔltaE pCDF{Ptrc}[hGLYAT2(co_Ec)-
fadD_Ec]{Placuv5}[alkLmod1] as biocatalyst.

| Fermentation | | | | | | |
|---|---|---|---|---|---|---|
| time [h] | C_{Lauroyl glycinate} [g/l] | C_{Myristoyl glycinate} [g/l] | C_{Palmitoyl glycinate} [g/l] | C_{Glycine} [g/l] | C_{Octanoic acid} [g/l] | C_{Decanoic acid} [g/l] |
| 47 | 9.7 | 2.9 | 1.3 | 0.0 | 0.0 | 0.0 |
| 65 | 8.5 | 2.6 | 1.2 | 0.0 | 0.0 | 0.0 |

| Fermentation | | | | | | |
|---|---|---|---|---|---|---|
| time [h] | C_{Lauric acid} [g/l] | C_{Myristic acid} [g/l] | C_{Palmitoleic acid} [g/l] | C_{Palmitic acid} [g/l] | C_{Oleic acid} [g/l] | C_{Stearic acid} [g/l] |
| 47 | 0.0 | 0.0 | 0.0 | 0.0 | 0.0 | n.d. |
| 65 | 0.0 | 0.0 | 0.0 | 0.0 | 0.0 | n.d. |

TABLE 20
Concentrations of different Acyl glycinates, free fatty acids and glycine after 47 h and 65 h of
biotransformation using E. coli W3110 ΔfadE ΔglyA pCDF{Ptrc}[hGLYAT2(co_Ec)-
fadD_Ec]{Placuv5}[alkLmod1 as biocatalyst.

| Fermentation | | | | | | |
|---|---|---|---|---|---|---|
| time [h] | C_{Lauroyl glycinate} [g/l] | C_{Myristoyl glycinate} [g/l] | C_{Palmitoyl glycinate} [g/l] | C_{Glycine} [g/l] | C_{Octanoic acid} [g/l] | C_{Decanoic acid} [g/l] |
| 47 | 10 | 3 | 1.3 | 9.5 | 0.0 | 0.0 |
| 65 | 8.5 | 2.5 | 1.1 | 8.6 | 0.0 | 0.0 |

| Fermentation | | | | | | |
|---|---|---|---|---|---|---|
| time [h] | C_{Lauric acid} [g/l] | C_{Myristic acid} [g/l] | C_{Palmitoleic acid} [g/l] | C_{Palmitic acid} [g/l] | C_{Oleic acid} [g/l] | C_{Stearic acid} [g/l] |
| 47 | 0.0 | 0.0 | 0.0 | 0.0 | 0.0 | n.d. |
| 65 | 0.0 | 0.0 | 0.0 | 0.0 | 0.0 | n.d. |

It was shown, that the ΔgcvTHP mutation reduces the glycine loss caused by the natural metabolism of E. coli significantly as already shown in Example 13. The glycine concentration at the end of the biotransformation is >7 g/L (respectively >8 g/L) for the mutants as shown in Table 17 (respectively Table 18). For E. coli lacking this mutation the glycine concentration at the end of the biotransformation is 0 g/L as shown in Table 16.

An additional mutation blocking a second glycine metabolising pathway (ΔItaE) does not enhance the effect of the ΔgcvTHP mutation. Surprisingly suppresses ΔItaE the desired effect of ΔgcvTHP as shown in Table 19.

It was shown, that the ΔglyA mutation also reduces the glycine loss caused by the natural metabolism of E. coli significantly. The glycine concentration at the end of the biotransformation is >8 g/L for the mutant as shown in Table 20.

### Example 26

Production of Acyl Glycinate by E. coli Strains from Hydrolysed Coconut Oil and Glycine Using the Alternative Porine FadL

The E. coli W3110 strains as generated in Example 12 and 23,
ΔfadE ΔgcvTHP pCDF{Ptac}[hGLYAT2(co_Ec)-fadD_Ec]
ΔfadE ΔgcvTHP pCDF{Ptac}[hGLYAT2(co_Ec)-fadD_Ec]{Placuv5}[fadL]
   were grown and biotransformed as described in Example 24. The conversion samples were taken after 47 h and 65 h of biotransformation and analysed as described in Example 7. Then results are shown in Tables 21 and 22.

TABLE 21
Concentrations of different Acyl glycinates, free fatty acids and glycine after 47 h and 65 h of
biotransformation using E. coli W3110 ΔfadE ΔgcvTHP pCDF{Ptac}[hGLYAT2(co_Ec)-fadD_Ec] as biocatalyst.

| Fermentation | | | | | | |
|---|---|---|---|---|---|---|
| time [h] | C_{Lauroyl glycinate} [g/l] | C_{Myristoyl glycinate} [g/l] | C_{Palmitoyl glycinate} [g/l] | C_{Glycine} [g/l] | C_{Octanoic acid} [g/l] | C_{Decanoic acid} [g/l] |
| 47 | 4.4 | 0.5 | 0.0 | 12.7 | 0.1 | 0.0 |
| 65 | 3.9 | 0.4 | 0.0 | 10.8 | 0.0 | 0.0 |

| Fermentation | | | | | | |
|---|---|---|---|---|---|---|
| time [h] | C_{Lauric acid} [g/l] | C_{Myristic acid} [g/l] | C_{Palmitoleic acid} [g/l] | C_{Palmitic acid} [g/l] | C_{Oleic acid} [g/l] | C_{Stearic acid} [g/l] |
| 47 | 3.4 | 2.0 | 0.0 | 1.0 | 1.0 | n.d. |
| 65 | 3.3 | 1.9 | 0.0 | 0.8 | 0.9 | n.d. |

TABLE 22
Concentrations of different Acyl glycinates, free fatty acids and glycine after 47 h and 65 h of
biotransformation using E. coli W3110 ΔfadE ΔgcvTHP pCDF{Ptac}[hGLYAT2(co_Ec)-
fadD_Ec]{Placuv5}[fadL] as biocatalyst.

| Fermentation | | | | | | |
|---|---|---|---|---|---|---|
| time [h] | C_{Lauroyl glycinate} [g/l] | C_{Myristoyl glycinate} [g/l] | C_{Palmitoyl glycinate} [g/l] | C_{Glycine} [g/l] | C_{Octanoic acid} [g/l] | C_{Decanoic acid} [g/l] |
| 47 | 7.8 | 2.4 | 1.1 | 9.4 | 0.0 | 0.0 |
| 65 | 7.2 | 2.2 | 1.0 | 7.8 | 0.0 | 0.0 |

| Fermentation | | | | | | |
|---|---|---|---|---|---|---|
| time [h] | C_{Lauric acid} [g/l] | C_{Myristic acid} [g/l] | C_{Palmitoleic acid} [g/l] | C_{Palmitic acid} [g/l] | C_{Oleic acid} [g/l] | C_{Stearic acid} [g/l] |
| 47 | 0.8 | 0.2 | 0.0 | 0.0 | 0.2 | n.d. |
| 65 | 0.5 | 0.1 | 0.0 | 0.0 | 0.0 | n.d. |

It was shown that a porine is important for good biocatalyst performances. No porine like in the strain E. coli W3110 ΔfadE ΔgcvTHP pCDF{Ptac}[hGLYAT2(co_Ec)-fadD_Ec] leads to significant amounts of residual fatty acids in the fermentation broth at the end of biotransformation carried out under chosen conditions (Table 21). AlkL used as a porine led to almost full conversion of all the fatty acids from hydrolized coconut oil (Table 17). It was also shown that FadL can be used as a porine like AlkL leading to almost full conversion of fatty acids subjected to biotransformation.

### REFERENCES

Allgemeine Mikrobiologie, 2008, Georg Thieme Verlag.
F. M. Ausubel (1995), Current Protocols in Molecular Biology. John Wiley & Sons, Inc.
Antonenkov, V., D. Van Veldhoven, P., P., Waelkens, E., and Mannaerts, G. P. (1997)
Arthur Lesk (2008), Introduction to bioinformatics, 3^{rd} edition.
Beguin, P and Aubert, J-P (1994) FEMS Microbial. Rev. 13: 25-58.
Brosius J, Erfle M and J Storella (1985). J Biol Chem. Mar 25; 260(6):3539-41
Cornish-Bowden (1995), Fundamentals of Enzyme Kinetics, Portland Press Limited, 1995.
Fujita, Y., Matsuoka, H., and Hirooka, K. (2007) Mol. Microbiology 66(4), 829-839.
Jeremy M Berg, John L Tymoczko, and Luber′ Stryer, Biochemistry, 5th edition, W. H. Freeman, 2002.
Kang, Y (2010), PLOS ONE 5 (10), e13557.
Katoh et al., Genome Information, 16(1), 22-33, 2005.
Liebl et al. (1991) International Journal of Systematic Bacteriology 41: 255-260.
Link A J, Phillips D, Church G M. J. Bacteriol. 1997. 179(20)
Ohima, K. et al. (1997) Biotechnol. Genet. Eng. Rev. 14: 365414.
Sambrook/Fritsch/Maniatis (1989): Molecular cloning—A Laboratory Manual, Cold Spring Harbour Press, 2^{nd} edition,
“The DIG System Users Guide for Filter Hybridization”, Boehringer Mannheim GmbH, Mannheim, Germany, 1993
Thompson et al., (1994) Nucleic Acids Research 22, 4637-4680.
Waluk, D. P., Schultz, N., and Hunt, M. C. (2010), FASEB J. 24, 2795-2803.
EP11191520.3, U.S. Pat. No. 5,734,070, US20140051136, GB1483500. U.S. Pat. No. 6,960,455

1-15. (canceled)

16. A cell for producing acyl glycinates, wherein said cell is genetically modified to comprise:
a) at least a first genetic mutation that, relative to the wild type cell, increases the expression of an amino acid-N-acyl-transferase;
b) at least a second genetic mutation that, relative to the wild type cell, increases the expression of an acyl-CoA synthetase; and
c) at least a third genetic mutation that, relative to the wild type cell, decreases the expression of at least one enzyme selected from the group consisting of: an enzyme of the glycine cleavage system; glycine hydroxymethyltransferase (GlyA); threonine aldolase (LtaE); threonine dehydrogenase (Tdh); 2-Amino-3-Ketobutyrate CoA-Ligase (Kbl); and allothreonine dehydrogenase (YdfG).

17. The cell of claim 16, wherein said cell comprises a mutation in an enzyme from the glycine cleavage system selected from the group consisting of: glycine cleavage system T protein; glycine cleavage system H protein; and glycine cleavage system P protein.

18. The cell of claim 16, wherein the third genetic mutation decreases the expression relative to the wild type cell of the glycine hydroxymethyltransferase (GlyA), the threonine aldolase (LtaE), the glycine cleavage system T protein, the glycine cleavage system H protein and the glycine cleavage system P protein.

19. The cell of claim 18, wherein the glycine cleavage system T protein has 85% sequence identity to SEQ ID NO:58, the glycine cleavage system H protein has 85% sequence identity to SEQ ID NO:59, and the glycine cleavage system P protein has 85% sequence identity to SEQ ID NO:60.

20. The cell of claim 16, wherein the glycine hydroxymethyltransferase (GlyA) has 85% sequence identity to SEQ ID NO:61 and the threonine aldolase (LtaE) has 85% sequence identity to SEQ ID NO:62.

21. The cell of claim 16, wherein the cell has a reduced fatty acid degradation capacity relative to the wild type cell.

22. The cell of claim 21, wherein the reduced fatty acid degradation capacity is a result of a decrease in expression, relative to the wild type cell, of at least one enzyme selected from the group consisting of: acyl-CoA dehydrogenase; 2,4-dienoyl-CoA reductase; enoyl-CoA hydratase; and 3-ketoacyl-CoA thiolase.

23. The cell of claim 16, wherein the amino acid-N-acyl-transferase has 85% sequence identity to SEQ ID NO:63 or SEQ ID NO:64 and the acyl-CoA synthetase has 85% sequence identity to SEQ ID NO:65.

24. The cell according to claim 16, wherein said cell is a bacterial cell.

25. The cell of claim 16, wherein the cell is E. coli.

26. The cell of claim 16, wherein the cell is further genetically modified to comprise a fourth genetic mutation, wherein, compared to the wild type cell, said fourth mutation increases the expression of at least one transporter protein.

27. The cell of claim 26, wherein the transporter protein is selected from the group consisting of FadL and AlkL.

28. The cell of claim 16, wherein the acyl glycinate is lauroylglycinate.

29. The cell of claim 16, wherein the cell is capable of making proteinogenic amino acids or fatty acids.

30. The cell of claim 16, wherein said cell is a bacterium and:
a) the amino acid-N-acyl-transferase comprises the sequence of SEQ ID NO:63 or SEQ ID NO:64;
b) the acyl-CoA synthetase comprises the sequence of SEQ ID NO:65; and
c) the third genetic mutation decreases the expression of at least one enzyme selected from the group consisting of: a glycine cleavage system T protein comprising the sequence of SEQ ID NO:58; a glycine cleavage system H protein comprising the sequence of SEQ ID NO:59; a glycine cleavage system P protein comprising the of SEQ ID NO:60; a glycine hydroxymethyltransferase (GlyA) comprising the sequence of SEQ ID NO:61; and a threonine aldolase (LtaE) comprising the sequence of SEQ ID NO:62.

31. The cell of claim 30, wherein the cell is E. coli.

32. The cell of claim 30, wherein the cell has a reduced fatty acid degradation capacity relative to the wild type cell.

33. The cell of claim 30, wherein the cell is further genetically modified to comprise a fourth genetic mutation, wherein, compared to the wild type cell, said fourth mutation increases the expression of at least one transporter protein selected from the group consisting of FadL and AlkL.

34. The cell of claim 30, wherein the acyl glycinate is lauroylglycinate.

35. A method for producing acyl amino acids, comprising contacting an amino acid and a fatty acid or an acyl CoA thereof with the cell of claim 16.


SEQUENCE LISTING (82 sequences)

SEQ ID NO: 1 - DNA, 906 bp, Umbellularia californica
  misc_feature (1)..(906): synUcTE (an acyl-CoA thioesterase) gene (codon- optimized)
atgactctagagtggaaaccgaaaccaaaactgcctcaactgctggatgatcacttcggtctgcacggtctggtgtttcgtcgtactttcgcaattcgttcttatgaagtgggtccagatcgttctacctccatcctggccgtcatgaaccacatgcaggaagccaccctgaatcacgcgaaatctgttggtatcctgggtgatggtttcggcactactctggaaatgtctaaacgtgacctgatgtgggtagtgcgtcgcacccacgtagcagtagagcgctaccctacttggggtgacactgtggaagtcgagtgttggattggcgcgtccggtaacaatggtatgcgtcgcgattttctggtccgtgactgtaaaacgggcgaaatcctgacgcgttgcacctccctgagcgttctgatgaacacccgcactcgtcgcctgtctaccatcccggacgaagtgcgcggtgagatcggtcctgctttcatcgataacgtggcagttaaagacgacgaaatcaagaaactgcaaaaactgaacgactccaccgcggactacatccagggcggtctgactccgcgctggaacgacctggatgttaatcagcatgtgaacaacctgaaatacgttgcttgggtcttcgagactgtgccggacagcattttcgaaagccatcacatttcctcttttactctggagtaccgtcgcgaatgtactcgcgactccgttctgcgcagcctgaccaccgtaagcggcggttctagcgaggcaggtctggtctgcgaccatctgctgcaactggaaggcggctccgaagtcctgcgtgcgcgtacggagtggcgtccaaagctgacggattctttccgcggcatctccgtaattccggcggaacctcgtgtttaa

SEQ ID NO: 2 - DNA, 159 bp, Artificial Sequence
  tac promotor for synUcTE expression
ggatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggagg

SEQ ID NO: 3 - DNA, 4977 bp, Artificial Sequence
  Vector pJ294[Ptac-synUcTE]
accaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgcccttattcgactctatagtgaagttcctattctctagaaagtataggaacttctgaagtgggggcggccgcaaattcagtaagcagaaagtcaaaagcctccgaccggaggcttttgactaaagcttaaacacgaggttccgccggaattacggagatgccgcggaaagaatccgtcagctttggacgccactccgtacgcgcacgcaggacttcggagccgccttccagttgcagcagatggtcgcagaccagacctgcctcgctagaaccgccgcttacggtggtcaggctgcgcagaacggagtcgcgagtacattcgcgacggtactccagagtaaaagaggaaatgtgatggctttcgaaaatgctgtccggcacagtctcgaagacccaagcaacgtatttcaggttgttcacatgctgattaacatccaggtcgttccagcgcggagtcagaccgccctggatgtagtccgcggtggagtcgttcagtttttgcagtttcttgatttcgtcgtctttaactgccacgttatcgatgaaagcaggaccgatctcaccgcgcacttcgtccgggatggtagacaggcgacgagtgcgggtgttcatcagaacgctcagggaggtgcaacgcgtcaggatttcgcccgttttacagtcacggaccagaaaatcgcgacgcataccattgttaccggacgcgccaatccaacactcgacttccacagtgtcaccccaagtagggtagcgctctactgctacgtgggtgcgacgcactacccacatcaggtcacgtttagacatttccagagtagtgccgaaaccatcacccaggataccaacagatttcgcgtgattcagggtggcttcctgcatgtggttcatgacggccaggatggaggtagaacgatctggacccacttcataagaacgaattgcgaaagtacgacgaaacaccagaccgtgcagaccgaagtgatcatccagcagttgaggcagttttggtttcggtttccactctagagtcattttataatcctccttgtaaacaaaattatttctagagggaaaccgttgtggaattgtgagcgctcacaattccacatccacacattatacgagccgatgattaattgtcaacagcgtcgatcactgtgcatgaagctcgtaattgttatccgctcacaattggatccaaaatgaagggaagttcctatactttctagagaataggaacttctatagggagtcgaataagggcgacacaaaaggtattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccaggatgcctggcagttccctactctcgccgctgcgctcggtcgttcggctgcgggacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatattcaccaccctgaattgactctcttccgggcgctatcatgccataccgcgaaaggttttgcgccattcgatggcgcgccgctttt

SEQ ID NO: 4 - DNA, 882 bp, Homo sapiens
  misc_feature (1)..(882): hGLYAT2 (an amino acid N-acyl transferase) codon optimised for E.coli
atgctggttctgcacaacagccaaaaactgcaaattctgtacaaatctctggaaaaatctattcctgaaagcattaaagtttatggtgcgatcttcaacatcaaggataaaaaccctttcaatatggaggtgctggtcgacgcgtggcctgattaccaaatcgtcatcacccgtccgcagaagcaagaaatgaaagatgaccaggaccactatactaacacctaccacatcttcactaaagcgccggacaaactggaagaagttctgagctactccaatgttatctcctgggaacaaacgctgcagattcaaggttgccaggaaggtctggatgaggcgattcgtaaggtcgctacctctaagtccgttcaagtcgattacatgaaaaccatcctgtttatcccggagctgccgaagaaacacaaaacctcctctaacgacaagatggagctgttcgaagtagacgatgacaacaaagagggtaacttttccaatatgttcctggacgcctcccacgccggtctggttaatgagcactgggcgttcggtaaaaacgaacgctccctgaagtacattgagcgttgtctgcaggattttctgggttttggtgtcctgggtccagagggtcaactggtctcttggatcgttatggaacagtcttgtgaactgcgtatgggttatactgtgccaaaataccgtcaccaaggtaatatgctgcaaatcggttatcatctggagaagtacctgagccagaaggaaatcccgttctatttccatgttgccgataataacgaaaagagcctgcaagccctgaacaatctgggcttcaagatctgcccttgcggttggcaccagtggaagtgcacgcctaaaaagtactgt

SEQ ID NO: 5 - DNA, 864 bp, Homo sapiens
  misc_feature (1)..(864): hGLYAT3 (another amino acid N-acyl transferase) (codon optimised for E.coli)
atgctggttctgaattgctctactaaactgctgatcctggagaaaatgctgaaatcctgtttcccggaatctctgaaagtttatggtgcggtaatgaatatcaaccgtggtaatccgtttcaaaaagaagttgttctggactcttggccagatttcaaggcggttatcacccgtcgtcagcgtgaagcggaaaccgacaacctggaccactataccaacgcgtatgcggttttctataaagatgttcgcgcgtaccgtcagctgctggaagaatgcgatgttttcaactgggaccaggtttttcagatccaaggtctgcaatccgagctgtacgacgtttctaaggccgtggcgaactctaaacaactgaacatcaagctgacctctttcaaagcggttcatttctctccggtatccagcctgccggacacctccttcctgaaaggtccgtctccgcgtctgacctacctgtctgttgcgaatgcggatctgctgaaccgtacttggtcccgtggtggcaacgaacagtgcctgcgttacattgctaacctgatctcttgcttcccgagcgtctgcgttcgtgatgaaaaaggtaacccagtatcttggtctatcaccgaccagttcgcgaccatgtgtcatggctacaccctgcctgaacaccgtcgtaagggttactctcgtctggttgcgctgaccctggcgcgtaagctgcagtctcgtggttttccgtcccagggtaacgtcctggacgacaacactgcgtccatctccctgctgaagtccctgcatgccgaattcctgccgtgccgtttccaccgtctgattctgaccccagcgaccttctctggtctgccgcacctg

SEQ ID NO: 6 - DNA, 1686 bp, Escherichia coli
  misc_feature (1)..(1686): fadD (an acyl-CoA synthetase)
ttgaagaaggtttggcttaaccgttatcccgcggacgttccgacggagatcaaccctgaccgttatcaatctctggtagatatgtttgagcagtcggtcgcgcgctacgccgatcaacctgcgtttgtgaatatgggggaggtaatgaccttccgcaagctggaagaacgcagtcgcgcgtttgccgcttatttgcaacaagggttggggctgaagaaaggcgatcgcgttgcgttgatgatgcctaatttattgcaatatccggtggcgctgtttggcattttgcgtgccgggatgatcgtcgtaaacgttaacccgttgtataccccgcgtgagcttgagcatcagcttaacgatagcggcgcatcggcgattgttatcgtgtctaactttgctcacacactggaaaaagtggttgataaaaccgccgttcagcacgtaattctgacccgtatgggcgatcagctatctacggcaaaaggcacggtagtcaatttcgttgttaaatacatcaagcgtttggtgccgaaataccatctgccagatgccatttcatttcgtagcgcactgcataacggctaccggatgcagtacgtcaaacccgaactggtgccggaagatttagcttttctgcaatacaccggcggcaccactggtgtggcgaaaggcgcgatgctgactcaccgcaatatgctggcgaacctggaacaggttaacgcgacctatggtccgctgttgcatccgggcaaagagctggtggtgacggcgctgccgctgtatcacatttttgccctgaccattaactgcctgctgtttatcgaactgggtgggcagaacctgcttatcactaacccgcgcgatattccagggttggtaaaagagttagcgaaatatccgtttaccgctatcacgggcgttaacaccttgttcaatgcgttgctgaacaataaagagttccagcagctggatttctccagtctgcatctttccgcaggcggtgggatgccagtgcagcaagtggtggcagagcgttgggtgaaactgaccggacagtatctgctggaaggctatggccttaccgagtgtgcgccgctggtcagcgttaacccatatgatattgattatcatagtggtagcatcggtttgccggtgccgtcgacggaagccaaactggtggatgatgatgataatgaagtaccaccaggtcaaccgggtgagctttgtgtcaaaggaccgcaggtgatgctgggttactggcagcgtcccgatgctaccgatgaaatcatcaaaaatggctggttacacaccggcgacatcgcggtaatggatgaagaaggattcctgcgcattgtcgatcgtaaaaaagacatgattctggtttccggttttaacgtctatcccaacgagattgaagatgtcgtcatgcagcatcctggcgtacaggaagtcgcggctgttggcgtaccttccggctccagtggtgaagcggtgaaaatcttcgtagtgaaaaaagatccatcgcttaccgaagagtcactggtgactttttgccgccgtcagctcacgggatacaaagtaccgaagctggtggagtttcgtgatgagttaccgaaatctaacgtcggaaaaattttgcgacgagaattacgtgacgaagcgcgcggcaaagtggacaataaagcctga

SEQ ID NO: 7 - DNA, 7138 bp, artificial sequence
  Vector pCDF[atfA1_Ab(co_Ec)-fadD_Ec]
cgggatctcgacgctctcccttatgcgactcctgcgtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataccttttgtgtcgcccttattcgactccctatagaagttcctattctctagaaagtataggaacttcccttcattttggatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggaggtaaaacatatgaaagcgctgtccccggtggatcagctgttcctgtggctggagaaacgtcagcaaccgatgcatgttggtggtctgcagctgtttagcttcccagagggtgcgggtcctaagtacgttagcgagttggctcagcaaatgcgtgactactgtcacccggtggccccgttcaatcagcgcctgacccgtcgcctgggtcaatactattggacgcgcgacaaacaatttgacattgaccaccattttcgccatgaagcgctgccgaagccgggtcgcattcgcgaactgctgtccttggttagcgcggagcacagcaatctgttggaccgcgagcgccctatgtgggaagcgcacctgattgagggcatccgtggccgccaattcgctttgtactataagatccaccatagcgtgatggatggcatcagcgctatgcgcattgcgtctaaaacgctgagcaccgacccgtctgagcgcgaaatggcaccagcatgggccttcaataccaagaaacgtagccgtagcctgccttccaatccagttgatatggcgagcagcatggcccgtctgacggcttctatcagcaaacaggcggcgaccgttccgggtctggctcgtgaagtgtacaaggtcacccagaaagctaagaaggatgagaactacgtgagcatttttcaagcgccggataccatcttgaacaacaccattaccggttcccgtcgttttgcagcacagagctttccgctgccgcgtctgaaagttatcgcgaaggcgtataactgcaccatcaacaccgtcgtcctgtctatgtgtggccacgcgctgcgtgaatatctgattagccaacacgcactgccggatgagccgctgatcgcgatggtgccgatgagcctgcgtcaggacgacagcacgggtggtaatcagattggcatgatcctggccaacctgggcacccatatctgcgatccggcgaatcgtttgcgtgttattcacgactccgtcgaggaagcgaaatcgcgtttcagccagatgagcccggaggagatcctgaactttaccgcactgactatggccccgacgggcttgaatctgctgaccggcctggcaccgaaatggcgtgcgttcaacgtcgtcattagcaacattccgggtccgaaggaaccgctgtattggaatggcgcccagctgcaaggtgtgtacccggtaagcattgcgttggaccgtattgcactgaacatcacgctgactagctacgtggatcagatggagttcggtctgatcgcatgccgtcgcacgctgccgagcatgcaacgtctgctggattatctggaacaaagcattcgtgagttggaaatcggtgccggcatcaagtaagcgttaagtctgagaggtgaagaattgaagaaggtttggcttaaccgttatcccgcggacgttccgacggagatcaaccctgaccgttatcaatctctggtagatatgtttgagcagtcggtcgcgcgctacgccgatcaacctgcgtttgtgaatatgggggaggtaatgaccttccgcaagctggaagaacgcagtcgcgcgtttgccgcttatttgcaacaagggttggggctgaagaaaggcgatcgcgttgcgttgatgatgcctaatttattgcaatatccggtggcgctgtttggcattttgcgtgccgggatgatcgtcgtaaacgttaacccgttgtataccccgcgtgagcttgagcatcagcttaacgatagcggcgcatcggcgattgttatcgtgtctaactttgctcacacactggaaaaagtggttgataaaaccgccgttcagcacgtaattctgacccgtatgggcgatcagctatctacggcaaaaggcacggtagtcaatttcgttgttaaatacatcaagcgtttggtgccgaaataccatctgccagatgccatttcatttcgtagcgcactgcataacggctaccggatgcagtacgtcaaacccgaactggtgccggaagatttagcttttctgcaatacaccggcggcaccactggtgtggcgaaaggcgcgatgctgactcaccgcaatatgctggcgaacctggaacaggttaacgcgacctatggtccgctgttgcatccgggcaaagagctggtggtgacggcgctgccgctgtatcacatttttgccctgaccattaactgcctgctgtttatcgaactgggtgggcagaacctgcttatcactaacccgcgcgatattccagggttggtaaaagagttagcgaaatatccgtttaccgctatcacgggcgttaacaccttgttcaatgcgttgctgaacaataaagagttccagcagctggatttctccagtctgcatctttccgcaggcggtgggatgccagtgcagcaagtggtggcagagcgttgggtgaaactgaccggacagtatctgctggaaggctatggccttaccgagtgtgcgccgctggtcagcgttaacccatatgatattgattatcatagtggtagcatcggtttgccggtgccgtcgacggaagccaaactggtggatgatgatgataatgaagtaccaccaggtcaaccgggtgagctttgtgtcaaaggaccgcaggtgatgctgggttactggcagcgtcccgatgctaccgatgaaatcatcaaaaatggctggttacacaccggcgacatcgcggtaatggatgaagaaggattcctgcgcattgtcgatcgtaaaaaagacatgattctggtttccggttttaacgtctatcccaacgagattgaagatgtcgtcatgcagcatcctggcgtacaggaagtcgcggctgttggcgtaccttccggctccagtggtgaagcggtgaaaatcttcgtagtgaaaaaagatccatcgcttaccgaagagtcactggtgactttttgccgccgtcagctcacgggatacaaagtaccgaagctggtggagtttcgtgatgagttaccgaaatctaacgtcggaaaaattttgcgacgagaattacgtgacgaagcgcgcggcaaagtggacaataaagcctgagcgttaagtcagtcgtcagacgccggttaatccggcgttttttttgacgcccactaaagagaaaacaatctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtc

SEQ ID NO: 8 - DNA, 6649 bp, artificial sequence
  Vector pCDF{Ptac}[hGLYAT2(co_Ec)-fadD_Ec]
cgggatctcgacgctctcccttatgcgactcctgcgtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataccttttgtgtcgcccttattcgactccctatagaagttcctattctctagaaagtataggaacttcccttcattttggatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggaggtaaaacatatgctggttctgcacaacagccaaaaactgcaaattctgtacaaatctctggaaaaatctattcctgaaagcattaaagtttatggtgcgatcttcaacatcaaggataaaaaccctttcaatatggaggtgctggtcgacgcgtggcctgattaccaaatcgtcatcacccgtccgcagaagcaagaaatgaaagatgaccaggaccactatactaacacctaccacatcttcactaaagcgccggacaaactggaagaagttctgagctactccaatgttatctcctgggaacaaacgctgcagattcaaggttgccaggaaggtctggatgaggcgattcgtaaggtcgctacctctaagtccgttcaagtcgattacatgaaaaccatcctgtttatcccggagctgccgaagaaacacaaaacctcctctaacgacaagatggagctgttcgaagtagacgatgacaacaaagagggtaacttttccaatatgttcctggacgcctcccacgccggtctggttaatgagcactgggcgttcggtaaaaacgaacgctccctgaagtacattgagcgttgtctgcaggattttctgggttttggtgtcctgggtccagagggtcaactggtctcttggatcgttatggaacagtcttgtgaactgcgtatgggttatactgtgccaaaataccgtcaccaaggtaatatgctgcaaatcggttatcatctggagaagtacctgagccagaaggaaatcccgttctatttccatgttgccgataataacgaaaagagcctgcaagccctgaacaatctgggcttcaagatctgcccttgcggttggcaccagtggaagtgcacgcctaaaaagtactgttaagcgttaagtctgagaggtgaagaattgaagaaggtttggcttaaccgttatcccgcggacgttccgacggagatcaaccctgaccgttatcaatctctggtagatatgtttgagcagtcggtcgcgcgctacgccgatcaacctgcgtttgtgaatatgggggaggtaatgaccttccgcaagctggaagaacgcagtcgcgcgtttgccgcttatttgcaacaagggttggggctgaagaaaggcgatcgcgttgcgttgatgatgcctaatttattgcaatatccggtggcgctgtttggcattttgcgtgccgggatgatcgtcgtaaacgttaacccgttgtataccccgcgtgagcttgagcatcagcttaacgatagcggcgcatcggcgattgttatcgtgtctaactttgctcacacactggaaaaagtggttgataaaaccgccgttcagcacgtaattctgacccgtatgggcgatcagctatctacggcaaaaggcacggtagtcaatttcgttgttaaatacatcaagcgtttggtgccgaaataccatctgccagatgccatttcatttcgtagcgcactgcataacggctaccggatgcagtacgtcaaacccgaactggtgccggaagatttagcttttctgcaatacaccggcggcaccactggtgtggcgaaaggcgcgatgctgactcaccgcaatatgctggcgaacctggaacaggttaacgcgacctatggtccgctgttgcatccgggcaaagagctggtggtgacggcgctgccgctgtatcacatttttgccctgaccattaactgcctgctgtttatcgaactgggtgggcagaacctgcttatcactaacccgcgcgatattccagggttggtaaaagagttagcgaaatatccgtttaccgctatcacgggcgttaacaccttgttcaatgcgttgctgaacaataaagagttccagcagctggatttctccagtctgcatctttccgcaggcggtgggatgccagtgcagcaagtggtggcagagcgttgggtgaaactgaccggacagtatctgctggaaggctatggccttaccgagtgtgcgccgctggtcagcgttaacccatatgatattgattatcatagtggtagcatcggtttgccggtgccgtcgacggaagccaaactggtggatgatgatgataatgaagtaccaccaggtcaaccgggtgagctttgtgtcaaaggaccgcaggtgatgctgggttactggcagcgtcccgatgctaccgatgaaatcatcaaaaatggctggttacacaccggcgacatcgcggtaatggatgaagaaggattcctgcgcattgtcgatcgtaaaaaagacatgattctggtttccggttttaacgtctatcccaacgagattgaagatgtcgtcatgcagcatcctggcgtacaggaagtcgcggctgttggcgtaccttccggctccagtggtgaagcggtgaaaatcttcgtagtgaaaaaagatccatcgcttaccgaagagtcactggtgactttttgccgccgtcagctcacgggatacaaagtaccgaagctggtggagtttcgtgatgagttaccgaaatctaacgtcggaaaaattttgcgacgagaattacgtgacgaagcgcgcggcaaagtggacaataaagcctgagcgttaagtcagtcgtcagacgccggttaatccggcgttttttttgacgcccactaaagagaaaacaatctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtc

SEQ ID NO: 9 - DNA, 6631 bp, Artificial Sequence
  Vector pCDF{Ptac}[hGLYAT3(co_Ec)-fadD_Ec]
cgggatctcgacgctctcccttatgcgactcctgcgtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataccttttgtgtcgcccttattcgactccctatagaagttcctattctctagaaagtataggaacttcccttcattttggatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggaggtaaaacatatgctggttctgaattgctctactaaactgctgatcctggagaaaatgctgaaatcctgtttcccggaatctctgaaagtttatggtgcggtaatgaatatcaaccgtggtaatccgtttcaaaaagaagttgttctggactcttggccagatttcaaggcggttatcacccgtcgtcagcgtgaagcggaaaccgacaacctggaccactataccaacgcgtatgcggttttctataaagatgttcgcgcgtaccgtcagctgctggaagaatgcgatgttttcaactgggaccaggtttttcagatccaaggtctgcaatccgagctgtacgacgtttctaaggccgtggcgaactctaaacaactgaacatcaagctgacctctttcaaagcggttcatttctctccggtatccagcctgccggacacctccttcctgaaaggtccgtctccgcgtctgacctacctgtctgttgcgaatgcggatctgctgaaccgtacttggtcccgtggtggcaacgaacagtgcctgcgttacattgctaacctgatctcttgcttcccgagcgtctgcgttcgtgatgaaaaaggtaacccagtatcttggtctatcaccgaccagttcgcgaccatgtgtcatggctacaccctgcctgaacaccgtcgtaagggttactctcgtctggttgcgctgaccctggcgcgtaagctgcagtctcgtggttttccgtcccagggtaacgtcctggacgacaacactgcgtccatctccctgctgaagtccctgcatgccgaattcctgccgtgccgtttccaccgtctgattctgaccccagcgaccttctctggtctgccgcacctgtaagcgttaagtctgagaggtgaagaattgaagaaggtttggcttaaccgttatcccgcggacgttccgacggagatcaaccctgaccgttatcaatctctggtagatatgtttgagcagtcggtcgcgcgctacgccgatcaacctgcgtttgtgaatatgggggaggtaatgaccttccgcaagctggaagaacgcagtcgcgcgtttgccgcttatttgcaacaagggttggggctgaagaaaggcgatcgcgttgcgttgatgatgcctaatttattgcaatatccggtggcgctgtttggcattttgcgtgccgggatgatcgtcgtaaacgttaacccgttgtataccccgcgtgagcttgagcatcagcttaacgatagcggcgcatcggcgattgttatcgtgtctaactttgctcacacactggaaaaagtggttgataaaaccgccgttcagcacgtaattctgacccgtatgggcgatcagctatctacggcaaaaggcacggtagtcaatttcgttgttaaatacatcaagcgtttggtgccgaaataccatctgccagatgccatttcatttcgtagcgcactgcataacggctaccggatgcagtacgtcaaacccgaactggtgccggaagatttagcttttctgcaatacaccggcggcaccactggtgtggcgaaaggcgcgatgctgactcaccgcaatatgctggcgaacctggaacaggttaacgcgacctatggtccgctgttgcatccgggcaaagagctggtggtgacggcgctgccgctgtatcacatttttgccctgaccattaactgcctgctgtttatcgaactgggtgggcagaacctgcttatcactaacccgcgcgatattccagggttggtaaaagagttagcgaaatatccgtttaccgctatcacgggcgttaacaccttgttcaatgcgttgctgaacaataaagagttccagcagctggatttctccagtctgcatctttccgcaggcggtgggatgccagtgcagcaagtggtggcagagcgttgggtgaaactgaccggacagtatctgctggaaggctatggccttaccgagtgtgcgccgctggtcagcgttaacccatatgatattgattatcatagtggtagcatcggtttgccggtgccgtcgacggaagccaaactggtggatgatgatgataatgaagtaccaccaggtcaaccgggtgagctttgtgtcaaaggaccgcaggtgatgctgggttactggcagcgtcccgatgctaccgatgaaatcatcaaaaatggctggttacacaccggcgacatcgcggtaatggatgaagaaggattcctgcgcattgtcgatcgtaaaaaagacatgattctggtttccggttttaacgtctatcccaacgagattgaagatgtcgtcatgcagcatcctggcgtacaggaagtcgcggctgttggcgtaccttccggctccagtggtgaagcggtgaaaatcttcgtagtgaaaaaagatccatcgcttaccgaagagtcactggtgactttttgccgccgtcagctcacgggatacaaagtaccgaagctggtggagtttcgtgatgagttaccgaaatctaacgtcggaaaaattttgcgacgagaattacgtgacgaagcgcgcggcaaagtggacaataaagcctgagcgttaagtcagtcgtcagacgccggttaatccggcgttttttttgacgcccactaaagagaaaacaatctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtc

SEQ ID NO: 10 - DNA, 693 bp, Pseudomonas putida
  misc_feature (1)..(693): alkL (an importer facilitating transport of hydrophobic acyl across cell membranes) gene
gtgagtttttctaattataaagtaatcgcgatgccggtgttggttgctaattttgttttgggggcggccactgcatgggcgaatgaaaattatccggcgaaatctgctggctataatcagggtgactgggtcgctagcttcaatttttctaaggtctatgtgggtgaggagcttggcgatctaaatgttggagggggggctttgccaaatgctgatgtaagtattggtaatgatacaacacttacgtttgatatcgcctattttgttagctcaaatatagcggtggatttttttgttggggtgccagctagggctaaatttcaaggtgagaaatcaatctcctcgctgggaagagtcagtgaagttgattacggccctgcaattctttcgcttcaatatcattacgatagctttgagcgactttatccatatgttggggttggtgttggtcgggtgctattttttgataaaaccgacggtgctttgagttcgtttgatattaaggataaatgggcgcctgcttttcaggttggccttagatatgaccttggtaactcatggatgctaaattcagatgtgcgttatattcctttcaaaacggacgtcacaggtactcttggcccggttcctgtttctactaaaattgaggttgatcctttcattctcagtcttggtgcgtcatatgttttctaa

SEQ ID NO: 11 - DNA, 153 bp, Artificial Sequence
  lacuv5 promoter
ggcagtgagcgcaacgcaattaatgtaagttagctcactcattaggcaccccaggcttgacactttatgcttccggctcgtataatgtgtggaattgtgagcggataacaataacaatttcacacaggatctaggaaccaaggagagtggcat

SEQ ID NO: 12 - DNA, 4876 bp, Artificial Sequence
  Vector pCDF[alkLmod1]
ggggaattgtgagcggataacaattcccctgtagaaataattttgtttaactttaataaggagatataccatgggcagcagccatcaccatcatcaccacagccaggatccttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgcccttattcgactctatagtgaagttcctattctctagaaagtataggaacttctgaagtggggatccggccggcctgcagccccgcagggcctgtctcggtcgatcattcagcccggctcatagatatgcgggcagtgagcgcaacgcaattaatgtaagttagctcactcattaggcaccccaggcttgacactttatgcttccggctcgtataatgtgtggaattgtgagcggataacaataacaatttcacacaggatctaggaaccaaggagagtggcatgtgagtttttctaattataaagtaatcgcgatgccggtgttggttgctaattttgttttgggggcggccactgcatgggcgaatgaaaattatccggcgaaatctgctggctataatcagggtgactgggtcgctagcttcaatttttctaaggtctatgtgggtgaggagcttggcgatctaaatgttggagggggggctttgccaaatgctgatgtaagtattggtaatgatacaacacttacgtttgatatcgcctattttgttagctcaaatatagcggtggatttttttgttggggtgccagctagggctaaatttcaaggtgagaaatcaatctcctcgctgggaagagtcagtgaagttgattacggccctgcaattctttcgcttcaatatcattacgatagctttgagcgactttatccatatgttggggttggtgttggtcgggtgctattttttgataaaaccgacggtgctttgagttcgtttgatattaaggataaatgggcgcctgcttttcaggttggccttagatatgaccttggtaactcatggatgctaaattcagatgtgcgttatattcctttcaaaacggacgtcacaggtactcttggcccggttcctgtttctactaaaattgaggttgatcctttcattctcagtcttggtgcgtcatatgttttctaaggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 13 - DNA, 7468 bp, Artificial Sequence
  Vector pCDF{Ptac}[hGLYAT2(co_Ec)-fadD_Ec] {Placuv5}[alkLmod1]
cgggatctcgacgctctcccttatgcgactcctgcgtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcattgtctcggtcgatcattcagcccggctcatagatatgcgggcagtgagcgcaacgcaattaatgtaagttagctcactcattaggcaccccaggcttgacactttatgcttccggctcgtataatgtgtggaattgtgagcggataacaataacaatttcacacaggatctaggaaccaaggagagtggcatgtgagtttttctaattataaagtaatcgcgatgccggtgttggttgctaattttgttttgggggcggccactgcatgggcgaatgaaaattatccggcgaaatctgctggctataatcagggtgactgggtcgctagcttcaatttttctaaggtctatgtgggtgaggagcttggcgatctaaatgttggagggggggctttgccaaatgctgatgtaagtattggtaatgatacaacacttacgtttgatatcgcctattttgttagctcaaatatagcggtggatttttttgttggggtgccagctagggctaaatttcaaggtgagaaatcaatctcctcgctgggaagagtcagtgaagttgattacggccctgcaattctttcgcttcaatatcattacgatagctttgagcgactttatccatatgttggggttggtgttggtcgggtgctattttttgataaaaccgacggtgctttgagttcgtttgatattaaggataaatgggcgcctgcttttcaggttggccttagatatgaccttggtaactcatggatgctaaattcagatgtgcgttatattcctttcaaaacggacgtcacaggtactcttggcccggttcctgtttctactaaaattgaggttgatcctttcattctcagtcttggtgcgtcatatgttttctaaggtaccctcgagtctggtggatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggaggtaaaacatatgctggttctgcacaacagccaaaaactgcaaattctgtacaaatctctggaaaaatctattcctgaaagcattaaagtttatggtgcgatcttcaacatcaaggataaaaaccctttcaatatggaggtgctggtcgacgcgtggcctgattaccaaatcgtcatcacccgtccgcagaagcaagaaatgaaagatgaccaggaccactatactaacacctaccacatcttcactaaagcgccggacaaactggaagaagttctgagctactccaatgttatctcctgggaacaaacgctgcagattcaaggttgccaggaaggtctggatgaggcgattcgtaaggtcgctacctctaagtccgttcaagtcgattacatgaaaaccatcctgtttatcccggagctgccgaagaaacacaaaacctcctctaacgacaagatggagctgttcgaagtagacgatgacaacaaagagggtaacttttccaatatgttcctggacgcctcccacgccggtctggttaatgagcactgggcgttcggtaaaaacgaacgctccctgaagtacattgagcgttgtctgcaggattttctgggttttggtgtcctgggtccagagggtcaactggtctcttggatcgttatggaacagtcttgtgaactgcgtatgggttatactgtgccaaaataccgtcaccaaggtaatatgctgcaaatcggttatcatctggagaagtacctgagccagaaggaaatcccgttctatttccatgttgccgataataacgaaaagagcctgcaagccctgaacaatctgggcttcaagatctgcccttgcggttggcaccagtggaagtgcacgcctaaaaagtactgttaagcgttaagtctgagaggtgaagaattgaagaaggtttggcttaaccgttatcccgcggacgttccgacggagatcaaccctgaccgttatcaatctctggtagatatgtttgagcagtcggtcgcgcgctacgccgatcaacctgcgtttgtgaatatgggggaggtaatgaccttccgcaagctggaagaacgcagtcgcgcgtttgccgcttatttgcaacaagggttggggctgaagaaaggcgatcgcgttgcgttgatgatgcctaatttattgcaatatccggtggcgctgtttggcattttgcgtgccgggatgatcgtcgtaaacgttaacccgttgtataccccgcgtgagcttgagcatcagcttaacgatagcggcgcatcggcgattgttatcgtgtctaactttgctcacacactggaaaaagtggttgataaaaccgccgttcagcacgtaattctgacccgtatgggcgatcagctatctacggcaaaaggcacggtagtcaatttcgttgttaaatacatcaagcgtttggtgccgaaataccatctgccagatgccatttcatttcgtagcgcactgcataacggctaccggatgcagtacgtcaaacccgaactggtgccggaagatttagcttttctgcaatacaccggcggcaccactggtgtggcgaaaggcgcgatgctgactcaccgcaatatgctggcgaacctggaacaggttaacgcgacctatggtccgctgttgcatccgggcaaagagctggtggtgacggcgctgccgctgtatcacatttttgccctgaccattaactgcctgctgtttatcgaactgggtgggcagaacctgcttatcactaacccgcgcgatattccagggttggtaaaagagttagcgaaatatccgtttaccgctatcacgggcgttaacaccttgttcaatgcgttgctgaacaataaagagttccagcagctggatttctccagtctgcatctttccgcaggcggtgggatgccagtgcagcaagtggtggcagagcgttgggtgaaactgaccggacagtatctgctggaaggctatggccttaccgagtgtgcgccgctggtcagcgttaacccatatgatattgattatcatagtggtagcatcggtttgccggtgccgtcgacggaagccaaactggtggatgatgatgataatgaagtaccaccaggtcaaccgggtgagctttgtgtcaaaggaccgcaggtgatgctgggttactggcagcgtcccgatgctaccgatgaaatcatcaaaaatggctggttacacaccggcgacatcgcggtaatggatgaagaaggattcctgcgcattgtcgatcgtaaaaaagacatgattctggtttccggttttaacgtctatcccaacgagattgaagatgtcgtcatgcagcatcctggcgtacaggaagtcgcggctgttggcgtaccttccggctccagtggtgaagcggtgaaaatcttcgtagtgaaaaaagatccatcgcttaccgaagagtcactggtgactttttgccgccgtcagctcacgggatacaaagtaccgaagctggtggagtttcgtgatgagttaccgaaatctaacgtcggaaaaattttgcgacgagaattacgtgacgaagcgcgcggcaaagtggacaataaagcctgagcgttaagtcagtcgtcagacgccggttaatccggcgttttttttgacgcccactaaagagaaaacaatctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtc

SEQ ID NO: 14 - DNA, 5367 bp, Artificial Sequence
  Vector pET-28b
ggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 15 - DNA, 6432 bp, Artificial Sequence
  Vector pET-28b{Ptac}[hGLYAT2(co_Ec)]
ggccgctctggtggatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggaggtaaaacatatgctggttctgcacaacagccaaaaactgcaaattctgtacaaatctctggaaaaatctattcctgaaagcattaaagtttatggtgcgatcttcaacatcaaggataaaaaccctttcaatatggaggtgctggtcgacgcgtggcctgattaccaaatcgtcatcacccgtccgcagaagcaagaaatgaaagatgaccaggaccactatactaacacctaccacatcttcactaaagcgccggacaaactggaagaagttctgagctactccaatgttatctcctgggaacaaacgctgcagattcaaggttgccaggaaggtctggatgaggcgattcgtaaggtcgctacctctaagtccgttcaagtcgattacatgaaaaccatcctgtttatcccggagctgccgaagaaacacaaaacctcctctaacgacaagatggagctgttcgaagtagacgatgacaacaaagagggtaacttttccaatatgttcctggacgcctcccacgccggtctggttaatgagcactgggcgttcggtaaaaacgaacgctccctgaagtacattgagcgttgtctgcaggattttctgggttttggtgtcctgggtccagagggtcaactggtctcttggatcgttatggaacagtcttgtgaactgcgtatgggttatactgtgccaaaataccgtcaccaaggtaatatgctgcaaatcggttatcatctggagaagtacctgagccagaaggaaatcccgttctatttccatgttgccgataataacgaaaagagcctgcaagccctgaacaatctgggcttcaagatctgcccttgcggttggcaccagtggaagtgcacgcctaaaaagtactgttaagcgttaagtctgagaggtgagagctcgaattcggatcccgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgc

SEQ ID NO: 16 - DNA, 6418 bp, Artificial Sequence
  pET-28b{Ptac}[hGLYAT3(co_Ec)]
ggccgcccttcattttggatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggaggtaaaacatatgctggttctgaattgctctactaaactgctgatcctggagaaaatgctgaaatcctgtttcccggaatctctgaaagtttatggtgcggtaatgaatatcaaccgtggtaatccgtttcaaaaagaagttgttctggactcttggccagatttcaaggcggttatcacccgtcgtcagcgtgaagcggaaaccgacaacctggaccactataccaacgcgtatgcggttttctataaagatgttcgcgcgtaccgtcagctgctggaagaatgcgatgttttcaactgggaccaggtttttcagatccaaggtctgcaatccgagctgtacgacgtttctaaggccgtggcgaactctaaacaactgaacatcaagctgacctctttcaaagcggttcatttctctccggtatccagcctgccggacacctccttcctgaaaggtccgtctccgcgtctgacctacctgtctgttgcgaatgcggatctgctgaaccgtacttggtcccgtggtggcaacgaacagtgcctgcgttacattgctaacctgatctcttgcttcccgagcgtctgcgttcgtgatgaaaaaggtaacccagtatcttggtctatcaccgaccagttcgcgaccatgtgtcatggctacaccctgcctgaacaccgtcgtaagggttactctcgtctggttgcgctgaccctggcgcgtaagctgcagtctcgtggttttccgtcccagggtaacgtcctggacgacaacactgcgtccatctccctgctgaagtccctgcatgccgaattcctgccgtgccgtttccaccgtctgattctgaccccagcgaccttctctggtctgccgcacctgtaagcgttaagtctgagaggtgagagctcgaattcggatcccgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgc

SEQ ID NO: 17 - DNA, 4334 bp, Escherichia coli
  misc_feature (1)..(4334): pBMT-3_ccdAB
ctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagtacgtacccggaattgccagctggggcgccctctggtaaggttgggaagccctgcaaagtaaactggatggctttcttgccgccaaggatctgatggcgcaggggatcaagctctgatcaagagacaggatgaggatcgtttcgatgaagcagcgtattacagtgacagttgacagcgacagctatcagttgctcaaggcatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagcgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatcctagacgaattctctagtagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccccctcaagtcaaaagcctccggtcggaggcttttgactttctgctatggaggtcaggtatgatttaaatggtcagtattgagcgatatctagagaattcgtcaacgaattcaagcttgatatcattcaggacgagcctcagactccagcgtaactggactgaaaacaaactaaagcgcccttgtggcgctttagttttgttccgctcatgataataatggtttcttagacgtcaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggtctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaag

SEQ ID NO: 18 - DNA, 5024 bp, Escherichia coli
  misc_feature (1)..(5024): pBMT-3_ccdAB_PT7-′tesA
caaaaaacccctcaagacccgtttagaggccccaaggggttatgctagttattgctcagcggtggcagcagcctaggttaattaagctgcgctagtagacgagtccatgtgctggcgttcaaatttcgcagcagcggtttctttaccagactcgaaaattcaggagtcgtggttgaccagcggctgcagttgcttggccatccagtcggcgatgaacggctgcgcgtcgcggttcgggtggatgccgtcgtcctgcatccactgcggcttcaggtagacttcctccatgaagaacggcagcagcggcacgtcgaattccttggccagcttcgggtagatcgcggagaaggcctcgttgtagcgacggccgtagttcgccggcaggcggatctgcatcagcagcggctcggcgttcgcggccttcacgtcctgcaggatctggcgcagcgtctgttcggtctgctgcggctggaagccgcgcaggccgtcgttgccgcccagctcgaccagcacccagcgcggctggtgctgcttcagcagcgccggcaggcgggccaggccctgctggctcgtgtcgccgctgatcgaggcgttgaccaccgaggtcttggactgccacttgtcgttcagcagcgccggccacgccgccgacgccgacatgcggtagcccgccgacagggagtcgcccaggatcagcagggtgtccgccgccatggtatatctccttattaaagttaaacaaaattatttctacaggggaattgttatccgctcacaattcccctatagtgagtcgtattaatttcctaatgcaggagtcgatcattcaggacgagcctcagactccagcgtaactggactgaaaacaaactaaagcgcccttgtggcgctttagttttgttccgctcatgataataatggtttcttagacgtcaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggtctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagtacgtacccggaattgccagctggggcgccctctggtaaggttgggaagccctgcaaagtaaactggatggctttcttgccgccaaggatctgatggcgcaggggatcaagctctgatcaagagacaggatgaggatcgtttcgatgaagcagcgtattacagtgacagttgacagcgacagctatcagttgctcaaggcatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagcgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatcctagacgaattctctagtagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaa

SEQ ID NO: 19 - DNA, 8268 bp, Escherichia coli
  misc_feature (1)..(8268): pBMT-3_ccdAB_PT7-nphT7-hbd-crt-ter
ccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagtacgtacccggaattgccagctggggcgccctctggtaaggttgggaagccctgcaaagtaaactggatggctttcttgccgccaaggatctgatggcgcaggggatcaagctctgatcaagagacaggatgaggatcgtttcgatgaagcagcgtattacagtgacagttgacagcgacagctatcagttgctcaaggcatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagcgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatcctagacgaattctctagtagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaaaaatcccttatgcgactcctgcattaggaaattaatacgactcactataggggaattgtgagcggataacaattcccctgtagaaataattttgtttaactttaataaggagatataccatgacggatgtccgctttcgtatcattggcacgggcgcttacgtcccggaacgcattgtcagcaacgacgaagtcggcgcaccggctggtgtggatgacgattggattacgcgtaaaaccggtatccgccagcgtcgctgggcggccgacgatcaagcaacgagcgatctggctaccgcagctggtcgtgcagcactgaaagcagctggcattacgccggaacagctgaccgtcatcgcagtggctacctctacgccggaccgtccgcagccgccgaccgcggcctatgttcaacatcacctgggtgcaaccggcacggcagcttttgatgttaacgctgtgtgcagcggtacggttttcgcgctgagctctgttgccggcaccctggtctatcgtggcggttacgcactggtcattggtgctgatctgtactcacgtatcctgaatccggcggaccgcaaaaccgtggttctgttcggcgatggtgcgggcgcgatggtgctgggcccgaccagcaccggcaccggtccgattgtgcgtcgcgttgcactgcatacgtttggcggtctgaccgatctgatccgtgttccggccggcggttcccgccagccgctggacaccgatggtctggacgcaggcctgcaatattttgcgatggatggccgcgaagtccgtcgcttcgtgaccgaacatctgccgcagctgattaaaggttttctgcacgaagcgggcgtggatgcggcggatattagccatttcgtgccgcaccaagccaacggtgtgatgctggacgaagtttttggcgaactgcatctgccgcgtgcaaccatgcaccgtacggtggaaacctacggtaatacgggcgcagctagtattccgatcacgatggatgcggccgttcgtgcaggttccttccgtccgggcgaactggttctgctggcgggctttggcggcggtatggcggcttcgtttgctctgattgaatggtgacctaatgcaggctgcaggcggatacgaggaggaataaaccatgaaaaaggtatgtgttataggtgcaggtactatgggttcaggaattgctcaggcatttgcagctaaaggatttgaagtagtattaagagatattaaagatgaatttgttgatagaggattagattttatcaataaaaatctttctaaattagttaaaaaaggaaagatagaagaagctactaaagttgaaatcttaactagaatttccggaacagttgaccttaatatggcagctgattgcgatttagttatagaagcagctgttgaaagaatggatattaaaaagcagatttttgctgacttagacaatatatgcaagccagaaacaattcttgcatcaaatacatcatcactttcaataacagaagtggcatcagcaactaaaagacctgataaggttataggtatgcatttctttaatccagctcctgttatgaagcttgtagaggtaataagaggaatagctacatcacaagaaacttttgatgcagttaaagagacatctatagcaataggaaaagatcctgtagaagtagcagaagcaccaggatttgttgtaaatagaatattaataccaatgattaatgaagcagttggtatattagcagaaggaatagcttcagtagaagacatagataaagctatgaaacttggagctaatcacccaatgggaccattagaattaggtgattttataggtcttgatatatgtcttgctataatggatgttttatactcagaaactggagattctaagtatagaccacatacattacttaagaagtatgtaagagcaggatggcttggaagaaaatcaggaaaaggtttctacgattattcaaaataagtttacaggatctgcagggaggaggaaatcatggagttgaacaacgttattctggagaaagaaggcaaggtggcggttgtcaccattaaccgtccaaaggccctgaacgctctgaactcggataccctgaaagagatggattacgttattggcgagattgagaatgacagcgaagtgctggctgtgattctgaccggtgcgggtgagaagagctttgtcgcgggtgcggacatcagcgagatgaaagaaatgaacaccatcgaaggccgtaagttcggtattctgggcaacaaggtgtttcgtcgtctggaactgctggagaaacctgtcattgctgccgtgaacggtttcgcgctgggcggtggttgcgagatcgctatgagctgcgatattcgtatcgcatcgtccaacgcacgctttggtcaaccggaggtcggtctgggtatcactccgggtttcggcggtacgcaacgtctgagccgcctggttggcatgggcatggcgaaacagttgattttcacggcacagaacattaaggcggatgaggcgctgcgtattggtctggtgaataaggtcgttgagccaagcgaactgatgaataccgcgaaagaaattgcgaacaagatcgttagcaatgccccggtggccgttaagctgtcgaaacaggcaatcaaccgtggcatgcagtgtgacatcgacaccgccctggcgtttgagagcgaggcgtttggtgagtgcttctccaccgaggaccaaaaggatgcgatgaccgcgttcattgagaaacgcaagatcgagggtttcaagaatcgttaatagaggaggataggaggttttcatatgattgtgaaaccgatggtccgtaataatatctgtctgaatgctcacccgcagggctgtaaaaaaggcgtggaagatcaaattgaatataccaaaaaacgtattacggcagaagtgaaagccggcgcaaaagctccgaaaaacgtgctggttctgggttgcagcaatggctatggtctggcttctcgcattaccgcggcctttggctacggtgcagctacgatcggcgttagtttcgaaaaagcaggttccgaaaccaaatatggcacgccgggttggtacaacaatctggcttttgatgaagcggccaaacgtgaaggcctgtatagtgtcaccattgatggtgacgcgttctccgatgaaattaaagcacaggtgatcgaagaagcgaagaaaaaaggcattaaatttgacctgatcgtttacagcctggcatctccggtccgtaccgatccggacacgggtatcatgcataaatctgtgctgaaaccgtttggcaaaaccttcacgggtaaaaccgttgatccgttcacgggcgaactgaaagaaattagcgcggaaccggccaacgatgaagaagcagctgcgaccgtcaaagtgatgggcggtgaagactgggaacgttggatcaaacagctgagtaaagaaggcctgctggaagaaggttgcattaccctggcgtattcctacatcggcccggaagcaacccaagctctgtatcgcaaaggcacgattggtaaagcgaaagaacatctggaagcgaccgcccaccgtctgaacaaagaaaatccgtcaatccgcgccttcgtttcggtcaataaaggtctggttacccgtgcatcagctgtgattccggttatcccgctgtacctggcatcgctgtttaaagtcatgaaagaaaaaggcaaccatgaaggttgtattgaacagatcacccgcctgtatgccgaacgtctgtaccgcaaagatggtacgattccggtggacgaagaaaatcgtattcgcatcgatgactgggaactggaagaagatgtccaaaaagccgtgagcgccctgatggaaaaagttaccggcgaaaacgcggaatctctgacggatctggccggttatcgtcacgactttctggcgagtaatggttttgatgttgaaggcattaactacgaagctgaagtggaacgctttgatcgcatttgatctagagaattcgtcaacgaattcaagcttgatatcattcaggacgagcctcagactccagcgtaactggactgaaaacaaactaaagcgcccttgtggcgctttagttttgttccgctcatgataataatggtttcttagacgtcaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggtctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctc

SEQ ID NO: 20 - DNA, 8959 bp, Escherichia coli
  misc_feature (1)..(8959): pBMT-3_ccdAB_PT7-′tesA_PT7-nphT7-hbd-crt-ter
ccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagtacgtacccggaattgccagctggggcgccctctggtaaggttgggaagccctgcaaagtaaactggatggctttcttgccgccaaggatctgatggcgcaggggatcaagctctgatcaagagacaggatgaggatcgtttcgatgaagcagcgtattacagtgacagttgacagcgacagctatcagttgctcaaggcatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagcgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatcctagacgaattctctagtagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatcttctcgacgctctcccttatgcgactcctgcattaggaaattaatacgactcactataggggaattgtgagcggataacaattcccctgtagaaataattttgtttaactttaataaggagatataccatggcggcggacaccctgctgatcctgggcgactccctgtcggcgggctaccgcatgtcggcgtcggcggcgtggccggcgctgctgaacgacaagtggcagtccaagacctcggtggtcaacgcctcgatcagcggcgacacgagccagcagggcctggcccgcctgccggcgctgctgaagcagcaccagccgcgctgggtgctggtcgagctgggcggcaacgacggcctgcgcggcttccagccgcagcagaccgaacagacgctgcgccagatcctgcaggacgtgaaggccgcgaacgccgagccgctgctgatgcagatccgcctgccggcgaactacggccgtcgctacaacgaggccttctccgcgatctacccgaagctggccaaggaattcgacgtgccgctgctgccgttcttcatggaggaagtctacctgaagccgcagtggatgcaggacgacggcatccacccgaaccgcgacgcgcagccgttcatcgccgactggatggccaagcaactgcagccgctggtcaaccacgactcctgaatttaaatcccttatgcgactcctgcattaggaaattaatacgactcactataggggaattgtgagcggataacaattcccctgtagaaataattttgtttaactttaataaggagatataccatgacggatgtccgctttcgtatcattggcacgggcgcttacgtcccggaacgcattgtcagcaacgacgaagtcggcgcaccggctggtgtggatgacgattggattacgcgtaaaaccggtatccgccagcgtcgctgggcggccgacgatcaagcaacgagcgatctggctaccgcagctggtcgtgcagcactgaaagcagctggcattacgccggaacagctgaccgtcatcgcagtggctacctctacgccggaccgtccgcagccgccgaccgcggcctatgttcaacatcacctgggtgcaaccggcacggcagcttttgatgttaacgctgtgtgcagcggtacggttttcgcgctgagctctgttgccggcaccctggtctatcgtggcggttacgcactggtcattggtgctgatctgtactcacgtatcctgaatccggcggaccgcaaaaccgtggttctgttcggcgatggtgcgggcgcgatggtgctgggcccgaccagcaccggcaccggtccgattgtgcgtcgcgttgcactgcatacgtttggcggtctgaccgatctgatccgtgttccggccggcggttcccgccagccgctggacaccgatggtctggacgcaggcctgcaatattttgcgatggatggccgcgaagtccgtcgcttcgtgaccgaacatctgccgcagctgattaaaggttttctgcacgaagcgggcgtggatgcggcggatattagccatttcgtgccgcaccaagccaacggtgtgatgctggacgaagtttttggcgaactgcatctgccgcgtgcaaccatgcaccgtacggtggaaacctacggtaatacgggcgcagctagtattccgatcacgatggatgcggccgttcgtgcaggttccttccgtccgggcgaactggttctgctggcgggctttggcggcggtatggcggcttcgtttgctctgattgaatggtgacctaatgcaggctgcaggcggatacgaggaggaataaaccatgaaaaaggtatgtgttataggtgcaggtactatgggttcaggaattgctcaggcatttgcagctaaaggatttgaagtagtattaagagatattaaagatgaatttgttgatagaggattagattttatcaataaaaatctttctaaattagttaaaaaaggaaagatagaagaagctactaaagttgaaatcttaactagaatttccggaacagttgaccttaatatggcagctgattgcgatttagttatagaagcagctgttgaaagaatggatattaaaaagcagatttttgctgacttagacaatatatgcaagccagaaacaattcttgcatcaaatacatcatcactttcaataacagaagtggcatcagcaactaaaagacctgataaggttataggtatgcatttctttaatccagctcctgttatgaagcttgtagaggtaataagaggaatagctacatcacaagaaacttttgatgcagttaaagagacatctatagcaataggaaaagatcctgtagaagtagcagaagcaccaggatttgttgtaaatagaatattaataccaatgattaatgaagcagttggtatattagcagaaggaatagcttcagtagaagacatagataaagctatgaaacttggagctaatcacccaatgggaccattagaattaggtgattttataggtcttgatatatgtcttgctataatggatgttttatactcagaaactggagattctaagtatagaccacatacattacttaagaagtatgtaagagcaggatggcttggaagaaaatcaggaaaaggtttctacgattattcaaaataagtttacaggatctgcagggaggaggaaatcatggagttgaacaacgttattctggagaaagaaggcaaggtggcggttgtcaccattaaccgtccaaaggccctgaacgctctgaactcggataccctgaaagagatggattacgttattggcgagattgagaatgacagcgaagtgctggctgtgattctgaccggtgcgggtgagaagagctttgtcgcgggtgcggacatcagcgagatgaaagaaatgaacaccatcgaaggccgtaagttcggtattctgggcaacaaggtgtttcgtcgtctggaactgctggagaaacctgtcattgctgccgtgaacggtttcgcgctgggcggtggttgcgagatcgctatgagctgcgatattcgtatcgcatcgtccaacgcacgctttggtcaaccggaggtcggtctgggtatcactccgggtttcggcggtacgcaacgtctgagccgcctggttggcatgggcatggcgaaacagttgattttcacggcacagaacattaaggcggatgaggcgctgcgtattggtctggtgaataaggtcgttgagccaagcgaactgatgaataccgcgaaagaaattgcgaacaagatcgttagcaatgccccggtggccgttaagctgtcgaaacaggcaatcaaccgtggcatgcagtgtgacatcgacaccgccctggcgtttgagagcgaggcgtttggtgagtgcttctccaccgaggaccaaaaggatgcgatgaccgcgttcattgagaaacgcaagatcgagggtttcaagaatcgttaatagaggaggataggaggttttcatatgattgtgaaaccgatggtccgtaataatatctgtctgaatgctcacccgcagggctgtaaaaaaggcgtggaagatcaaattgaatataccaaaaaacgtattacggcagaagtgaaagccggcgcaaaagctccgaaaaacgtgctggttctgggttgcagcaatggctatggtctggcttctcgcattaccgcggcctttggctacggtgcagctacgatcggcgttagtttcgaaaaagcaggttccgaaaccaaatatggcacgccgggttggtacaacaatctggcttttgatgaagcggccaaacgtgaaggcctgtatagtgtcaccattgatggtgacgcgttctccgatgaaattaaagcacaggtgatcgaagaagcgaagaaaaaaggcattaaatttgacctgatcgtttacagcctggcatctccggtccgtaccgatccggacacgggtatcatgcataaatctgtgctgaaaccgtttggcaaaaccttcacgggtaaaaccgttgatccgttcacgggcgaactgaaagaaattagcgcggaaccggccaacgatgaagaagcagctgcgaccgtcaaagtgatgggcggtgaagactgggaacgttggatcaaacagctgagtaaagaaggcctgctggaagaaggttgcattaccctggcgtattcctacatcggcccggaagcaacccaagctctgtatcgcaaaggcacgattggtaaagcgaaagaacatctggaagcgaccgcccaccgtctgaacaaagaaaatccgtcaatccgcgccttcgtttcggtcaataaaggtctggttacccgtgcatcagctgtgattccggttatcccgctgtacctggcatcgctgtttaaagtcatgaaagaaaaaggcaaccatgaaggttgtattgaacagatcacccgcctgtatgccgaacgtctgtaccgcaaagatggtacgattccggtggacgaagaaaatcgtattcgcatcgatgactgggaactggaagaagatgtccaaaaagccgtgagcgccctgatggaaaaagttaccggcgaaaacgcggaatctctgacggatctggccggttatcgtcacgactttctggcgagtaatggttttgatgttgaaggcattaactacgaagctgaagtggaacgctttgatcgcatttgatctagagaattcgtcaacgaattcaagcttgatatcattcaggacgagcctcagactccagcgtaactggactgaaaacaaactaaagcgcccttgtggcgctttagttttgttccgctcatgataataatggtttcttagacgtcaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggtctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctc

SEQ ID NO: 21 - DNA, 5367 bp, Escherichia coli; identical to SEQ ID NO: 14
  misc_feature (1)..(5367): pET-28b(empty vector)

SEQ ID NO: 22 - DNA, 43 bp, Artificial Sequence
  oligonucleotide o-MO-40 for gcvTHP amplification
ccggggatcgcggccgcggaccgaacttatccgccaggcaatg

SEQ ID NO: 23 - DNA, 51 bp, Artificial Sequence
  oligonucleotide o-MO-41 for gcvTHP amplification
ttactggtattcgctaatcggctcgagcatcttgtcctcattgaataagcg

SEQ ID NO: 24 - DNA, 30 bp, Artificial Sequence
  oligonucleotide o-MO-42 for gcvTHP amplification
ctcgagccgattagcgaataccagtaattc

SEQ ID NO: 25 - DNA, 41 bp, Artificial Sequence
  oligonucleotide o-MO-43 for gcvTHP amplification
ggatcgcggccgctctagagttcatcgtgctgatcgattgc

SEQ ID NO: 26 - DNA, 553 bp, Artificial Sequence
  PCR product 1 from example 12, 1553bp
ccggggatcgcggccgcggaccgaacttatccgccaggcaatgggattaaacgatttgcctgaatggctgcgttaaaaatttctcctctgttgtttatttgatacccatcacactttcatctcccggttttttcgccgggagattttcctcatttgaaataaactaatttcacctccgttttcgcattatattttctaatgccattattttttgatttagtgttttttgacatttttttagctcttaatattgtcttattcaaattgactttctcatcacatcatctttgtatagaaactggtgtattttttggttttttattctgtcgcgatttttgcattttttaaccataagctaatgtgatgatcaattttaccttatggttaacagtctgtttcggtggtaagttcaggcaaaagagaacgattgcgttggggaccgggagtggctccgatgctgggtttcgtggtgataatttcaccatgaaaaagttgtcagccccgcttattcaatgaggacaagatgctcgagccgattagcgaataccagtaa

SEQ ID NO: 27 - DNA, 547 bp, Artificial Sequence
  PCR product 2 from example 12, 1553bp
ctcgagccgattagcgaataccagtaattcactgattcgactattttctaaaggcgcttcggcgcctttttagtcagatgacaaagtacaaaagtgctcagacagtcccctcgcccctttggggagagggttagggtgaggggaacaggccggcactggcgcgaatatttaccctcaccccggccctctccctgaaagggcgagggggaaaagcgtgccaacattgaagattgagccagtttgttagcaatctcaaagatacgtcaacgaattaatttttctcggaaaaacaaatggctatagcacttgtgactggtggcagtcgcggcatcgggcgggcaactgcattactgttggcgcaagaagggtatacggtggcggttaattatcagcaaaacctccacgcggcgcaggaagtgatgaacttaataacgcaagccggtggcaaagcattcgtgctccaggcggatatcagcgacgaaaaccaggtcgttgcgatgtttacagcaatcgatcagcacgatgaactctagagcggccgcgatcc

SEQ ID NO: 28 - DNA, 5667 bp, Artificial Sequence
  vector pKO3
cctttcgtcttcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtctcggtacccggggatcgcggccgcggaccggatcctctagagcggccgcgatcctctagagtcgaccggtggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcaccgcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgagttcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatattgactaccggaagcagtgtgaccgtgtgcttctcaaatgcctgattcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcggggatcgacgctctcccttatgcgactcctgca

SEQ ID NO: 29 - DNA, 6693 bp, Artificial Sequence
  knock-out vector pKO3 delta gcvTHP
cctttcgtcttcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtctcggtacccggggatcgcggccgcggaccgaacttatccgccaggcaatgggattaaacgatttgcctgaatggctgcgttaaaaatttctcctctgttgtttatttgatacccatcacactttcatctcccggttttttcgccgggagattttcctcatttgaaataaactaatttcacctccgttttcgcattatattttctaatgccattattttttgatttagtgttttttgacatttttttagctcttaatattgtcttattcaaattgactttctcatcacatcatctttgtatagaaactggtgtattttttggttttttattctgtcgcgatttttgcattttttaaccataagctaatgtgatgatcaattttaccttatggttaacagtctgtttcggtggtaagttcaggcaaaagagaacgattgcgttggggaccgggagtggctccgatgctgggtttcgtggtgataatttcaccatgaaaaagttgtcagccccgcttattcaatgaggacaagatgctcgagccgattagcgaataccagtaattcactgattcgactattttctaaaggcgcttcggcgcctttttagtcagatgacaaagtacaaaagtgctcagacagtcccctcgcccctttggggagagggttagggtgaggggaacaggccggcactggcgcgaatatttaccctcaccccggccctctccctgaaagggcgagggggaaaagcgtgccaacattgaagattgagccagtttgttagcaatctcaaagatacgtcaacgaattaatttttctcggaaaaacaaatggctatagcacttgtgactggtggcagtcgcggcatcgggcgggcaactgcattactgttggcgcaagaagggtatacggtggcggttaattatcagcaaaacctccacgcggcgcaggaagtgatgaacttaataacgcaagccggtggcaaagcattcgtgctccaggcggatatcagcgacgaaaaccaggtcgttgcgatgtttacagcaatcgatcagcacgatgaactctagagcggccgcgatcctctagagtcgaccggtggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcaccgcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgagttcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatattgactaccggaagcagtgtgaccgtgtgcttctcaaatgcctgattcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcggggatcgacgctctcccttatgcgactcctgca

SEQ ID NO: 30 - DNA, 30 bp, Artificial Sequence
  DNA sequence after deletion of gcvTHP
atgctcgagccgattagcgaataccagtaa

SEQ ID NO: 31 - DNA, 38 bp, Artificial Sequence
  oligonucleotide o-MO-44 for amplifying the upstream region of glyA
gggatcgcggccgcggaccgagggcttcacgttgatcg

SEQ ID NO: 32 - DNA, 45 bp, Artificial Sequence
  oligonucleotide o-MO-45 for amplifying the upstream region of glyA
tatgcgtaaaccgggtaacgctcgagcatccgcatctcctgactc

SEQ ID NO: 33 - DNA, 22 bp, Artificial Sequence
  oligonucleotide o-MO-46 for amplifying the downstream region of glyA
cgttacccggtttacgcataag

SEQ ID NO: 34 - DNA, 39 bp, Artificial Sequence
  oligonucleotide o-MO-47 for amplifying the downstream region of glyA
ggatcgcggccgctctagagatcaccagctgggttgatc

SEQ ID NO: 35 - DNA, 546 bp, Artificial Sequence
  PCR product 1 from Example 14
gggatcgcggccgcggaccgagggcttcacgttgatcgccattacgctggttactcatgttaaaaatttctttgagttctgggttatgagtaaacatacggtcgtagaaatgggcggttaactttggccccgtttccaccagtaaagggatggtggcttttactgtagcgatggtttgagcgtcaagcatatggtcttcctttttttgcatcttaattgatgtatctcaaatgcatcttataaaaaatagccctgcaatgtaaatggttctttggtgtttttcagaaagaatgtgatgaagtgaaaaatttgcatcacaaacctgaaaagaaatccgtttccggttgcaagctctttattctccaaagccttgcgtagcctgaaggtaatcgtttgcgtaaattcctttgtcaagacctgttatcgcacaatgattcggttatactgttcgccgttgtccaacaggaccgcctataaaggccaaaaattttattgttagctgagtcaggagatgcggatgctcgagcgttacccggtttacgcata

SEQ ID NO: 36 - DNA, 520 bp, Artificial Sequence
  PCR product 2 from Example 14
cgttacccggtttacgcataagcgaaacggtgatttgctgtcaatgtgctcgttgttcatgccggatgcggcgtgaacgccttatccggcctacaaaactttgcaaattcaatatattgcaatctccgtgtaggcctgataagcgtagcgcatcaggcaatttttcgtttatgatcatcaaggcttccttcgggaagcctttctacgttatcgcgccatcaaatctgtcgtaactgcgcctcaacatacaaatagccaattcccagcacctgttgtgcgcggcttaattgcccaaagccaatttgcgtcgctttcacgctcggtccttcgtcgcggtcgaacgggttaaatcctgtctggcgaataatagtattaagccagttctcaccaaaggcacaactgcgaccgtacaaccaaatctgattaatattgaggatgttgaggaagttatacagactcaaaccaatggcattggcagagcgatcaacccagctggtgatctctagagcggccgcgatcc

SEQ ID NO: 37 - DNA, 26 bp, Artificial Sequence
  oligonucleotide o-MO-52
actgaggatccgggatcgcggccgcg

SEQ ID NO: 38 - DNA, 31 bp, Artificial Sequence
  oligonucleotide o-MO-53
actgaggatccggatcgcggccgctctagag

SEQ ID NO: 39 - DNA, 1052 bp, Artificial Sequence
  PCR product 3 from Example 14
gatccgggatcgcggccgcggaccgagggcttcacgttgatcgccattacgctggttactcatgttaaaaatttctttgagttctgggttatgagtaaacatacggtcgtagaaatgggcggttaactttggccccgtttccaccagtaaagggatggtggcttttactgtagcgatggtttgagcgtcaagcatatggtcttcctttttttgcatcttaattgatgtatctcaaatgcatcttataaaaaatagccctgcaatgtaaatggttctttggtgtttttcagaaagaatgtgatgaagtgaaaaatttgcatcacaaacctgaaaagaaatccgtttccggttgcaagctctttattctccaaagccttgcgtagcctgaaggtaatcgtttgcgtaaattcctttgtcaagacctgttatcgcacaatgattcggttatactgttcgccgttgtccaacaggaccgcctataaaggccaaaaattttattgttagctgagtcaggagatgcggatgctcgagcgttacccggtttacgcataagcgaaacggtgatttgctgtcaatgtgctcgttgttcatgccggatgcggcgtgaacgccttatccggcctacaaaactttgcaaattcaatatattgcaatctccgtgtaggcctgataagcgtagcgcatcaggcaatttttcgtttatgatcatcaaggcttccttcgggaagcctttctacgttatcgcgccatcaaatctgtcgtaactgcgcctcaacatacaaatagccaattcccagcacctgttgtgcgcggcttaattgcccaaagccaatttgcgtcgctttcacgctcggtccttcgtcgcggtcgaacgggttaaatcctgtctggcgaataatagtattaagccagttctcaccaaaggcacaactgcgaccgtacaaccaaatctgattaatattgaggatgttgaggaagttatacagactcaaaccaatggcattggcagagcgatcaacccagctggtgatctctagagcggccgcgatccg

SEQ ID NO: 40 - DNA, 6719 bp, Artificial Sequence
  pKO3 delta glyA
cctttcgtcttcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtctcggtacccggggatcgcggccgcggaccggatccgggatcgcggccgcggaccgagggcttcacgttgatcgccattacgctggttactcatgttaaaaatttctttgagttctgggttatgagtaaacatacggtcgtagaaatgggcggttaactttggccccgtttccaccagtaaagggatggtggcttttactgtagcgatggtttgagcgtcaagcatatggtcttcctttttttgcatcttaattgatgtatctcaaatgcatcttataaaaaatagccctgcaatgtaaatggttctttggtgtttttcagaaagaatgtgatgaagtgaaaaatttgcatcacaaacctgaaaagaaatccgtttccggttgcaagctctttattctccaaagccttgcgtagcctgaaggtaatcgtttgcgtaaattcctttgtcaagacctgttatcgcacaatgattcggttatactgttcgccgttgtccaacaggaccgcctataaaggccaaaaattttattgttagctgagtcaggagatgcggatgctcgagcgttacccggtttacgcataagcgaaacggtgatttgctgtcaatgtgctcgttgttcatgccggatgcggcgtgaacgccttatccggcctacaaaactttgcaaattcaatatattgcaatctccgtgtaggcctgataagcgtagcgcatcaggcaatttttcgtttatgatcatcaaggcttccttcgggaagcctttctacgttatcgcgccatcaaatctgtcgtaactgcgcctcaacatacaaatagccaattcccagcacctgttgtgcgcggcttaattgcccaaagccaatttgcgtcgctttcacgctcggtccttcgtcgcggtcgaacgggttaaatcctgtctggcgaataatagtattaagccagttctcaccaaaggcacaactgcgaccgtacaaccaaatctgattaatattgaggatgttgaggaagttatacagactcaaaccaatggcattggcagagcgatcaacccagctggtgatctctagagcggccgcgatccggatcctctagagcggccgcgatcctctagagtcgaccggtggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcaccgcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgagttcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatattgactaccggaagcagtgtgaccgtgtgcttctcaaatgcctgattcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcggggatcgacgctctcccttatgcgactcctgca

SEQ ID NO: 41 - DNA, 30 bp, Artificial Sequence
  DNA sequence after deletion of glyA
atgctcgagcgttacccggtttacgcataa

SEQ ID NO: 42 - DNA, 39 bp, Artificial Sequence
  ltaE-UP_fw
ggatcgcggccgcggaccgcaggcgacagagccagaacg

SEQ ID NO: 43 - DNA, 47 bp, Artificial Sequence
  ltAE-UP-XhoI_rev
ctctccttaacgcgccaggaactcgagcatggcatgtccttattatg

SEQ ID NO: 44 - DNA, 21 bp, Artificial Sequence
  ltaE-DOWN_fw
ttcctggcgcgttaaggagag

SEQ ID NO: 45 - DNA, 40 bp, Artificial Sequence
  ltaE-DOWN_rev
ggatcgcggccgctctagagtagaccatatcgcgcatgac

SEQ ID NO: 46 - DNA, 540 bp, Artificial Sequence
  PCR product 4 from Example 15
ggatcgcggccgcggaccgcaggcgacagagccagaacgtcaggtggtcgccatgtgcggcgatggcggttttagcatgttgatgggcgatttcctctcagtagtgcagatgaaactgccagtgaaaattgtcgtctttaacaacagcgtgctgggctttgtggcgatggagatgaaagctggtggctatttgactgacggcaccgaactacacgacacaaactttgcccgcattgccgaagcgtgcggcattacgggtatccgtgtagaaaaagcgtctgaagttgatgaagccctgcaacgcgccttctccatcgacggtccggtgttggtggatgtggtggtcgccaaagaagagttagccattccaccgcagatcaaactcgaacaggccaaaggtttcagcctgtatatgctgcgcgcaatcatcagcggacgcggtgatgaagtgatcgaactggcgaaaacaaactggctaaggtaaaaagggtggcatttcccgtcataataaggacatgccatgctcgagttcctggcgcg

SEQ ID NO: 47 - DNA, 536 bp, Artificial Sequence
  PCR product 5 from Example 15
ttcctggcgcgttaaggagagagacgtgccgcaacgcattttagttctcggtgccagtggctacattggtcagcatctggtgcgcacactcagccagcaagggcatcagatcctggcggcggcacgtcatgtcgacaggcttgcaaagctgcaactggcaaacgtcagttgccataaagtcgatctcagctggccggataacctaccggccctgttgcaggatatcgatacggtctattttctggtgcacagcatgggcgaaggcggcgattttatcgctcaggagcgccaggtggctctcaacgtccgcgatgcgctacgtgaagtaccagttaagcaattaatctttcttagttcgttgcaggccccgccacatgagcagtcggatcatctgcgtgctcgtcaggctacggcggacattctgcgtgaagcgaatgtacccgtgaccgaactgcgggccggaattatcgttggcgcaggttccgcggcgttcgaagtcatgcgcgatatggtctactctagagcggccgcgatcc

SEQ ID NO: 48 - DNA, 26 bp, Artificial Sequence
  oligonucleotide o-MO-54 from Example 15
actgaggatccggatcgcggccgcgg

SEQ ID NO: 49 - DNA, 29 bp, Artificial Sequence
  oligoneucleotide o-MO-55 from Example 15
actgaggatccggatcgcggccgctctag

SEQ ID NO: 50 - DNA, 1071 bp, Artificial Sequence
  PCR product 6 from Example 15
gatccggatcgcggccgcggaccgcaggcgacagagccagaacgtcaggtggtcgccatgtgcggcgatggcggttttagcatgttgatgggcgatttcctctcagtagtgcagatgaaactgccagtgaaaattgtcgtctttaacaacagcgtgctgggctttgtggcgatggagatgaaagctggtggctatttgactgacggcaccgaactacacgacacaaactttgcccgcattgccgaagcgtgcggcattacgggtatccgtgtagaaaaagcgtctgaagttgatgaagccctgcaacgcgccttctccatcgacggtccggtgttggtggatgtggtggtcgccaaagaagagttagccattccaccgcagatcaaactcgaacaggccaaaggtttcagcctgtatatgctgcgcgcaatcatcagcggacgcggtgatgaagtgatcgaactggcgaaaacaaactggctaaggtaaaaagggtggcatttcccgtcataataaggacatgccatgctcgagttcctggcgcgttaaggagagagacgtgccgcaacgcattttagttctcggtgccagtggctacattggtcagcatctggtgcgcacactcagccagcaagggcatcagatcctggcggcggcacgtcatgtcgacaggcttgcaaagctgcaactggcaaacgtcagttgccataaagtcgatctcagctggccggataacctaccggccctgttgcaggatatcgatacggtctattttctggtgcacagcatgggcgaaggcggcgattttatcgctcaggagcgccaggtggctctcaacgtccgcgatgcgctacgtgaagtaccagttaagcaattaatctttcttagttcgttgcaggccccgccacatgagcagtcggatcatctgcgtgctcgtcaggctacggcggacattctgcgtgaagcgaatgtacccgtgaccgaactgcgggccggaattatcgttggcgcaggttccgcggcgttcgaagtcatgcgcgatatggtctactctagagcggccgcgatccg

SEQ ID NO: 51 - DNA, 6738 bp, Artificial Sequence
  pKO3 delta ltaE
cctttcgtcttcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtctcggtacccggggatcgcggccgcggaccggatccggatcgcggccgcggaccgcaggcgacagagccagaacgtcaggtggtcgccatgtgcggcgatggcggttttagcatgttgatgggcgatttcctctcagtagtgcagatgaaactgccagtgaaaattgtcgtctttaacaacagcgtgctgggctttgtggcgatggagatgaaagctggtggctatttgactgacggcaccgaactacacgacacaaactttgcccgcattgccgaagcgtgcggcattacgggtatccgtgtagaaaaagcgtctgaagttgatgaagccctgcaacgcgccttctccatcgacggtccggtgttggtggatgtggtggtcgccaaagaagagttagccattccaccgcagatcaaactcgaacaggccaaaggtttcagcctgtatatgctgcgcgcaatcatcagcggacgcggtgatgaagtgatcgaactggcgaaaacaaactggctaaggtaaaaagggtggcatttcccgtcataataaggacatgccatgctcgagttcctggcgcgttaaggagagagacgtgccgcaacgcattttagttctcggtgccagtggctacattggtcagcatctggtgcgcacactcagccagcaagggcatcagatcctggcggcggcacgtcatgtcgacaggcttgcaaagctgcaactggcaaacgtcagttgccataaagtcgatctcagctggccggataacctaccggccctgttgcaggatatcgatacggtctattttctggtgcacagcatgggcgaaggcggcgattttatcgctcaggagcgccaggtggctctcaacgtccgcgatgcgctacgtgaagtaccagttaagcaattaatctttcttagttcgttgcaggccccgccacatgagcagtcggatcatctgcgtgctcgtcaggctacggcggacattctgcgtgaagcgaatgtacccgtgaccgaactgcgggccggaattatcgttggcgcaggttccgcggcgttcgaagtcatgcgcgatatggtctactctagagcggccgcgatccggatcctctagagcggccgcgatcctctagagtcgaccggtggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcaccgcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgagttcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatattgactaccggaagcagtgtgaccgtgtgcttctcaaatgcctgattcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcggggatcgacgctctcccttatgcgactcctgca

SEQ ID NO: 52 - DNA, 24 bp, Artificial Sequence
  DNA sequence after deletion of ltaE
atgctcgagttcctggcgcgttaa

SEQ ID NO: 53 - protein, 294 aa, Nomocharis pardanthina
MLVLHNAQKLQILYKSLEKSIPESIKVYGAIFNIKDKNPFNMEVLVDAWPDYQIVITRPQKQEMKDDQDHYTNTYHIFTKAPDKLEEVLSYSSVINWEQTLQIQGCQEGLDEAIRKAATSKSVQVDYMKTMLFIPELPKKHKTSSNEKMELFKVDDDNKEGNFSNMFLDASHAGLVNEHWAFGKNERSLKYIERCLQDFLGFGVLGPEGQLVSWIVMEQSCELRMGYTVPKYRHQGNMLQIGYHFEKYLSQKEIPFYFHVADNNEKSLQALKNLGFKICPCGWHQWKCTPKKYC

SEQ ID NO: 54 - protein, 297 aa, Saimiri boliviensis
MFVLHDTQILQTLYKSLEKRNPESIKVYGAIFNIKNKNPFNMEVLVDAWPDYQTVIIRPKKQEMKDDQDHYTNTYHIFTKAPDKLEEILSCSEVINWEQTFQIQGCQEGLNEAIRKVAASKTVQVDYVKTVLFKPELLKEHKTSSNDKMELFEMHKVDDNNKKRNFSSMFLDASHAGLINEHWAFGKNERSLKYTERCLQDFLGFGVLGPEGELISWVVMEQSCELRMGYTIPKYRNQRHMSQILCYLEKYLSQKEIPFYIHVADYNVAGLQIMKNMGYMICPCGWHQWKCTPKKYC

SEQ ID NO: 55 - protein, 298 aa, Felis catus
MFVLHESQKLHILYESLEKSIPESLKVYGAVFNIKNKNPFNMEVLVDAWPDYQIVITRPQREEMKDDLDHYTNTYQIFTKAPDKLEEVLAQPQLINWDQVFQIQGCQESLQEAIRKISASKSVRVDYVKTIRIMTEMPVKPNDDQVDVMESFKMPKVDDDTKKGSFQNISLDPSHAGLVNEQWAFGKNDRSLKYIERCLQNFPGFGVLGPEGRLISWIVMEQSCEMRMGYTVPEYRKHGYMKKTSVHLMNYVIQKKVPCYFHVSEHKEEYNQALRDLKFKAASCGWHQWKCSPEKYVD

SEQ ID NO: 56 - protein, 299 aa, Bos taurus
MFVLREPQQLQILYESLEESIPESLKVYGTLFHIRNKNPFNLEVLVDAWPEYQTVVIRPQKEEMKDDLDYYTNTYHIFTKAPDKLEEVLACPQVINWEQAFQIQGCQESLGEAIQKVAASKSVQVDYLRTVLFVFEKPTLEASSGDKMDLMKLLKIPKVLEDKRKETFQNIFLDVSHARLVNEHWELGKNEKSLKYVERCLQNFAGFGVLSSEGKPISWFLTEQSCEIRMGYTFPKYRGQGHMWQMGCHSIAYFFSKKIPFYIHAAEDKEKTQQTLLSFGFKNVLCGWHQWKCTPKKYC

SEQ ID NO: 57 - protein, 296 aa, Mus musculus
MIVPLQGAQMLQMLEKSLRKYLPESLKVYGTVYHMIHGNPFNLKALVDKWPDFNTVVVRPQEQEMTDDLDFYINTYQVYSKDPQNCQEFLESSEVINWKQHLQIQSSQSHLNKTIQNLASIQSFQIKHSENILYVSSETIKKLFPSLLDTKNLSTGSGKPKAIDQDKFKLSSLDVVHAALVNKFWLFGGNERSQRFIERCIKNFPSSCVLGPEGTPASWTLMDQTGEMRMGGTMPEYRLQGLVSFVVHSQDQIMTKRGYPVYSHTEKSNIAMQKMSYTLQHLPMPCAWNQWKCMPM

SEQ ID NO: 58 - protein, 364 aa, Artificial Sequence
  gcvT
MAQQTPLYEQHTLCGARMVDFHGWMMPLHYGSQIDEHHAVRTDAGMFDVSHMTIVDLRGSRTREFLRYLLANDVAKLTKSGKALYSGMLNASGGVIDDLIVYYFTEDFFRLVVNSATREKDLSWITQHAEPFGIEITVRDDLSMIAVQGPNAQAKAATLFNDAQRQAVEGMKPFFGVQAGDLFIATTGYTGEAGYEIALPNEKAADFWRALVEAGVKPCGLGARDTLRLEAGMNLYGQEMDETISPLAANMGWTIAWEPADRDFIGREALEVQREHGTEKLVGLVMTEKGVLRNELPVRFTDAQGNQHEGIITSGTFSPTLGYSIALARVPEGIGETAIVQIRNREMPVKVTKPVFVRNGKAVA

SEQ ID NO: 59 - protein, 129 aa, Artificial Sequence
  gcvH
MSNVPAELKYSKEHEWLRKEADGTYTVGITEHAQELLGDMVFVDLPEVGATVSAGDDCAVAESVKAASDIYAPVSGEIVAVNDALSDSPELVNSEPYAGGWIFKIKASDESELESLLDATAYEALLEDE

SEQ ID NO: 60 - protein, 957 aa, Artificial Sequence
  gcvP
MTQTLSQLENSGAFIERHIGPDAAQQQEMLNAVGAQSLNALTGQIVPKDIQLATPPQVGAPATEYAALAELKAIASRNKRFTSYIGMGYTAVQLPPVILRNMLENPGWYTAYTPYQPEVSQGRLEALLNFQQVTLDLTGLDMASASLLDEATAAAEAMAMAKRVSKLKNANRFFVASDVHPQTLDVVRTRAETFGFEVIVDDAQKVLDHQDVFGVLLQQVGTTGEIHDYTALISELKSRKIVVSVAADIMALVLLTAPGKQGADIVFGSAQRFGVPMGYGGPHAAFFAAKDEYKRSMPGRIIGVSKDAAGNTALRMAMQTREQHIRREKANSNICTSQVLLANIASLYAVYHGPVGLKRIANRIHRLTDILAAGLQQKGLKLRHAHYFDTLCVEVADKAGVLTRAEAAEINLRSDILNAVGITLDETTTRENVMQLFNVLLGDNHGLDIDTLDKDVAHDSRSIQPAMLRDDEILTHPVFNRYHSETEMMRYMHSLERKDLALNQAMIPLGSCTMKLNAAAEMIPITWPEFAELHPFCPPEQAEGYQQMIAQLADWLVKLTGYDAVCMQPNSGAQGEYAGLLAIRHYHESRNEGHRDICLIPASAHGTNPASAHMAGMQVVVVACDKNGNIDLTDLRAKAEQAGDNLSCIMVTYPSTHGVYEETIREVCEVVHQFGGQVYLDGANMNAQVGITSPGFIGADVSHLNLHKTFCIPHGGGGPGMGPIGVKAHLAPFVPGHSVVQIEGMLTRQGAVSAAPFGSASILPISWMYIRMMGAEGLKKASQVAILNANYIASRLQDAFPVLYTGRDGRVAHECILDIRPLKEETGISELDIAKRLIDYGFHAPTMSFPVAGTLMVEPTESESKVELDRFIDAMLAIRAEIDQVKAGVWPLEDNPLVNAPHIQSELVAEWAHPYSREVAVFPAGVADKYWPTVKRLDDVYGDRNLFCSCVPISEYQ

SEQ ID NO: 61 - protein, 417 aa, Artificial Sequence
  glyA
MLKREMNIADYDAELWQAMEQEKVRQEEHIELIASENYTSPRVMQAQGSQLTNKYAEGYPGKRYYGGCEYVDIVEQLAIDRAKELFGADYANVQPHSGSQANFAVYTALLEPGDTVLGMNLAHGGHLTHGSPVNFSGKLYNIVPYGIDATGHIDYADLEKQAKEHKPKMIIGGFSAYSGVVDWAKMREIADSIGAYLFVDMAHVAGLVAAGVYPNPVPHAHVVTTTTHKTLAGPRGGLILAKGGSEELYKKLNSAVFPGGQGGPLMHVIAGKAVALKEAMEPEFKTYQQQVAKNAKAMVEVFLERGYKVVSGGTDNHLFLVDLVDKNLTGKEADAALGRANITVNKNSVPNDPKSPFVTSGIRVGTPAITRRGFKEAEAKELAGWMCDVLDSINDEAVIERIKGKVLDICARYPVYA

SEQ ID NO: 62 - protein, 333 aa, Artificial Sequence
  ltaE
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENTHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 63 - protein, 294 aa, Homo sapiens
MLVLHNSQKLQILYKSLEKSIPESIKVYGAIFNIKDKNPFNMEVLVDAWPDYQIVITRPQKQEMKDDQDHYTNTYHIFTKAPDKLEEVLSYSNVISWEQTLQIQGCQEGLDEAIRKVATSKSVQVDYMKTILFIPELPKKHKTSSNDKMELFEVDDDNKEGNFSNMFLDASHAGLVNEHWAFGKNERSLKYIERCLQDFLGFGVLGPEGQLVSWIVMEQSCELRMGYTVPKYRHQGNMLQIGYHLEKYLSQKEIPFYFHVADNNEKSLQALNNLGFKICPCGWHQWKCTPKKYC

SEQ ID NO: 64 - protein, 288 aa, Homo sapiens
MLVLNCSTKLLILEKMLKSCFPESLKVYGAVMNINRGNPFQKEVVLDSWPDFKAVITRRQREAETDNLDHYTNAYAVFYKDVRAYRQLLEECDVFNWDQVFQIQGLQSELYDVSKAVANSKQLNIKLTSFKAVHFSPVSSLPDTSFLKGPSPRLTYLSVANADLLNRTWSRGGNEQCLRYIANLISCFPSVCVRDEKGNPVSWSITDQFATMCHGYTLPEHRRKGYSRLVALTLARKLQSRGFPSQGNVLDDNTASISLLKSLHAEFLPCRFHRLILTPATFSGLPHL

SEQ ID NO: 65 - protein, 561 aa, Homo sapiens
LKKVWLNRYPADVPTEINPDRYQSLVDMFEQSVARYADQPAFVNMGEVMTFRKLEERSRAFAAYLQQGLGLKKGDRVALMMPNLLQYPVALFGILRAGMIVVNVNPLYTPRELEHQLNDSGASAIVIVSNFAHTLEKVVDKTAVQHVILTRMGDQLSTAKGTVVNFVVKYIKRLVPKYHLPDAISFRSALHNGYRMQYVKPELVPEDLAFLQYTGGTTGVAKGAMLTHRNMLANLEQVNATYGPLLHPGKELVVTALPLYHIFALTINCLLFIELGGQNLLITNPRDIPGLVKELAKYPFTAITGVNTLFNALLNNKEFQQLDFSSLHLSAGGGMPVQQVVAERWVKLTGQYLLEGYGLTECAPLVSVNPYDIDYHSGSIGLPVPSTEAKLVDDDDNEVPPGQPGELCVKGPQVMLGYWQRPDATDEIIKNGWLHTGDIAVMDEEGFLRIVDRKKDMILVSGFNVYPNEIEDVVMQHPGVQEVAAVGVPSGSSGEAVKIFVVKKDPSLTEESLVTFCRRQLTGYKVPKLVEFRDELPKSNVGKILRRELRDEARGKVDNKA

SEQ ID NO: 66 - DNA, 29 bp, Artificial Sequence
  primer fadL_ec-fp
gagtggcatggtcatgagccagaaaaccc

SEQ ID NO: 67 - DNA, 54 bp, Artificial Sequence
  primer fadL_EC_rp
cacaattggatccaccagactcgagggtacctcagaacgcgtagttaaagttag

SEQ ID NO: 68 - DNA, 34 bp, Artificial Sequence
  primer upstream_fp
taagatgttatcagtatttattatgcattgtctc

SEQ ID NO: 69 - DNA, 31 bp, Artificial Sequence
  primer upstream_rp
ggctcatgaccatgccactctccttggttcc

SEQ ID NO: 70 - DNA, 8120 bp, Artificial Sequence
  vector pCDF{Ptac}[hGLYAT2(co_Ec)-fadD_Ec] {Placuv5}[fadL
gatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggaggtaaaacatatgctggttctgcacaacagccaaaaactgcaaattctgtacaaatctctggaaaaatctattcctgaaagcattaaagtttatggtgcgatcttcaacatcaaggataaaaaccctttcaatatggaggtgctggtcgacgcgtggcctgattaccaaatcgtcatcacccgtccgcagaagcaagaaatgaaagatgaccaggaccactatactaacacctaccacatcttcactaaagcgccggacaaactggaagaagttctgagctactccaatgttatctcctgggaacaaacgctgcagattcaaggttgccaggaaggtctggatgaggcgattcgtaaggtcgctacctctaagtccgttcaagtcgattacatgaaaaccatcctgtttatcccggagctgccgaagaaacacaaaacctcctctaacgacaagatggagctgttcgaagtagacgatgacaacaaagagggtaacttttccaatatgttcctggacgcctcccacgccggtctggttaatgagcactgggcgttcggtaaaaacgaacgctccctgaagtacattgagcgttgtctgcaggattttctgggttttggtgtcctgggtccagagggtcaactggtctcttggatcgttatggaacagtcttgtgaactgcgtatgggttatactgtgccaaaataccgtcaccaaggtaatatgctgcaaatcggttatcatctggagaagtacctgagccagaaggaaatcccgttctatttccatgttgccgataataacgaaaagagcctgcaagccctgaacaatctgggcttcaagatctgcccttgcggttggcaccagtggaagtgcacgcctaaaaagtactgttaagcgttaagtctgagaggtgaagaattgaagaaggtttggcttaaccgttatcccgcggacgttccgacggagatcaaccctgaccgttatcaatctctggtagatatgtttgagcagtcggtcgcgcgctacgccgatcaacctgcgtttgtgaatatgggggaggtaatgaccttccgcaagctggaagaacgcagtcgcgcgtttgccgcttatttgcaacaagggttggggctgaagaaaggcgatcgcgttgcgttgatgatgcctaatttattgcaatatccggtggcgctgtttggcattttgcgtgccgggatgatcgtcgtaaacgttaacccgttgtataccccgcgtgagcttgagcatcagcttaacgatagcggcgcatcggcgattgttatcgtgtctaactttgctcacacactggaaaaagtggttgataaaaccgccgttcagcacgtaattctgacccgtatgggcgatcagctatctacggcaaaaggcacggtagtcaatttcgttgttaaatacatcaagcgtttggtgccgaaataccatctgccagatgccatttcatttcgtagcgcactgcataacggctaccggatgcagtacgtcaaacccgaactggtgccggaagatttagcttttctgcaatacaccggcggcaccactggtgtggcgaaaggcgcgatgctgactcaccgcaatatgctggcgaacctggaacaggttaacgcgacctatggtccgctgttgcatccgggcaaagagctggtggtgacggcgctgccgctgtatcacatttttgccctgaccattaactgcctgctgtttatcgaactgggtgggcagaacctgcttatcactaacccgcgcgatattccagggttggtaaaagagttagcgaaatatccgtttaccgctatcacgggcgttaacaccttgttcaatgcgttgctgaacaataaagagttccagcagctggatttctccagtctgcatctttccgcaggcggtgggatgccagtgcagcaagtggtggcagagcgttgggtgaaactgaccggacagtatctgctggaaggctatggccttaccgagtgtgcgccgctggtcagcgttaacccatatgatattgattatcatagtggtagcatcggtttgccggtgccgtcgacggaagccaaactggtggatgatgatgataatgaagtaccaccaggtcaaccgggtgagctttgtgtcaaaggaccgcaggtgatgctgggttactggcagcgtcccgatgctaccgatgaaatcatcaaaaatggctggttacacaccggcgacatcgcggtaatggatgaagaaggattcctgcgcattgtcgatcgtaaaaaagacatgattctggtttccggttttaacgtctatcccaacgagattgaagatgtcgtcatgcagcatcctggcgtacaggaagtcgcggctgttggcgtaccttccggctccagtggtgaagcggtgaaaatcttcgtagtgaaaaaagatccatcgcttaccgaagagtcactggtgactttttgccgccgtcagctcacgggatacaaagtaccgaagctggtggagtttcgtgatgagttaccgaaatctaacgtcggaaaaattttgcgacgagaattacgtgacgaagcgcgcggcaaagtggacaataaagcctgagcgttaagtcagtcgtcagacgccggttaatccggcgttttttttgacgcccactaaagagaaaacaatctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcgtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcattgtctcggtcgatcattcagcccggctcatagatatgcgggcagtgagcgcaacgcaattaatgtaagttagctcactcattaggcaccccaggcttgacactttatgcttccggctcgtataatgtgtggaattgtgagcggataacaataacaatttcacacaggatctaggaaccaaggagagtggcatggtcatgagccagaaaaccctgtttacaaagtctgctctcgcagtcgcagtggcacttatctccacccaggcctggtcggcaggctttcagttaaacgaattttcttcctctggcctgggccgggcttattcaggggaaggcgcaattgccgatgatgcaggtaacgtcagccgtaaccccgcattgattactatgtttgaccgcccgacattttctgcgggtgcggtttatattgacccggatgtaaatatcagcggaacgtctccatctggtcgtagcctgaaagccgataacatcgcgcctacggcatgggttccgaacatgcactttgttgcaccgattaacgaccaatttggttggggcgcttctattacctctaactatggtctggctacagagtttaacgatacttatgcaggcggctctgtcgggggtacaaccgaccttgaaaccatgaacctgaacttaagcggtgcgtatcgcttaaataatgcatggagctttggtcttggtttcaacgccgtctacgctcgcgcgaaaattgaacgtttcgcaggcgatctggggcagttggttgctggccaaattatgcaatctcctgctggccaaactcagcaagggcaagcattggcagctaccgccaacggtattgacagtaataccaaaatcgctcatctgaacggtaaccagtggggctttggctggaacgccggaatcctgtatgaactggataaaaataaccgctatgcactgacctaccgttctgaagtgaaaattgacttcaaaggtaactacagcagcgatcttaatcgtgcgtttaataactacggtttgccaattcctaccgcgacaggtggcgcaacgcaatcgggttatctgacgctgaacctgcctgaaatgtgggaagtgtcaggttataaccgtgttgatccacagtgggcgattcactatagcctggcttacaccagctggagtcagttccagcagctgaaagcgacctcaaccagtggcgacacgctgttccagaaacatgaaggctttaaagatgcttaccgcatcgcgttgggtaccacttattactacgatgataactggaccttccgtaccggtatcgcctttgatgacagcccagttcctgcacagaatcgttctatctccattccggaccaggaccgtttctggctgagtgcaggtacgacttacgcatttaataaagatgcttcagtcgacgttggtgtttcttatatgcacggtcagagcgtgaaaattaacgaaggcccataccagttcgagtctgaaggtaaagcctggctgttcggtactaactttaactacgcgttctgaggtaccctcgagtctggtg

SEQ ID NO: 71 - DNA, 56 bp, Artificial Sequence
  primer 1960_up_fp
ctggtctcggtacccggggatcgcggccgcgaaagaattctataaattagttctgg

SEQ ID NO: 72 - DNA, 39 bp, Artificial Sequence
  primer 1960_up_rp
acgcccagtgcctcgagcattgcgattctccagacaggg

SEQ ID NO: 73 - DNA, 36 bp, Artificial Sequence
  primer 1960_down_fp
aatgctcgaggcactgggcgttatcgcctgaggatg

SEQ ID NO: 74 - DNA, 54 bp, Artificial Sequence
  primer 1960_down_rp
cgccaccggtcgactctagaggatcgcggccgccccaccagatcaaacgacagc

SEQ ID NO: 75 - DNA, 6680 bp, Artificial Sequence
  vector pKO3 delta kbl
cctttcgtcttcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtctcggtacccggggatcgcggccgcgaaagaattctataaattagttctggaaagtcaaaaaataacactaaataataacattgtggacgatgaccagggcatttttgtcatgtgctattacaaacgccccgatctcttcaacctaaactatctggggagaggaaaatggttcgatttgtttcgctgcttcaggagtaacactttaggggcaaaaatgcaggcgctgagaattttcctttcaagaaaataggtttccctccattagtgggtagcgttttgataccaactgagataggaaaaatgatgctttggcatcattttttctttttatatcaatgcagtaaaatcaatcatcattatgcccagccaaaactgtacagtttttctactatctgtaggtgttgagagacatcaagttacacgttatttatcctgaattttgcagaagtgttaacgcgttatctcgtcgcgacctataagtttgggtaatatgtgctggaatttgccctgtctggagaatcgcaatgctcgaggcactgggcgttatcgcctgaggatgtgagatgaaagcgttatccaaactgaaagcggaagagggcatctggatgaccgacgttcctgtaccggaactcgggcataacgatctgctgattaaaatccgtaaaacagccatctgcgggactgacgttcacatctataactgggatgagtggtcgcaaaaaaccatcccggtgccgatggtcgtgggccatgaatatgtcggtgaagtggtaggtattggtcaggaagtgaaaggcttcaagatcggcgatcgcgtttctggcgaaggccatatcacctgtggtcattgccgcaactgtcgtggtggtcgtacccatttgtgccgcaacacgataggcgttggtgttaatcgcccgggctgctttgccgaatatctggtgatcccggcattcaacgccttcaaaatccccgacaatatttccgatgacttagccgcaatttttgatcccttcggtaacgccgtgcataccgcgctgtcgtttgatctggtggggcggccgcgatcctctagagtcgaccggtggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcaccgcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgagttcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatattgactaccggaagcagtgtgaccgtgtgcttctcaaatgcctgattcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcggggatcgacgctctcccttatgcgactcctgca

SEQ ID NO: 76 - DNA, 30 bp, Escherichia coli
atgctcgaggcactgggcgttatcgcctga

SEQ ID NO: 77 - DNA, 7468 bp, Artificial Sequence; identical to SEQ ID NO: 13
  vector pCDF{Ptrc}[hGLYAT2(co_Ec)-fadD_Ec] {Placuv5}[alkLmod1]

SEQ ID NO: 78 - protein, 230 aa, Pseudomonas putida
VSFSNYKVIAMPVLVANFVLGAATAWANENYPAKSAGYNQGDWVASFNFSKVYVGEELGDLNVGGGALPNADVSIGNDTTLTFDIAYFVSSNIAVDFFVGVPARAKFQGEKSISSLGRVSEVDYGPAILSLQYHYDSFERLYPYVGVGVGRVLFFDKTDGALSSFDIKDKWAPAFQVGLRYDLGNSWMLNSDVRYIPFKTDVTGTLGPVPVSTKIEVDPFILSLGASYVF

SEQ ID NO: 79 - protein, 448 aa, Escherichia coli
MVMSQKTLFTKSALAVAVALISTQAWSAGFQLNEFSSSGLGRAYSGEGAIADDAGNVSRNPALITMFDRPTFSAGAVYIDPDVNISGTSPSGRSLKADNIAPTAWVPNMHFVAPINDQFGWGASITSNYGLATEFNDTYAGGSVGGTTDLETMNLNLSGAYRLNNAWSFGLGFNAVYARAKIERFAGDLGQLVAGQIMQSPAGQTQQGQALAATANGIDSNTKIAHLNGNQWGFGWNAGILYELDKNNRYALTYRSEVKIDFKGNYSSDLNRAFNNYGLPIPTATGGATQSGYLTLNLPEMWEVSGYNRVDPQWAIHYSLAYTSWSQFQQLKATSTSGDTLFQKHEGFKDAYRIALGTTYYYDDNWTFRTGIAFDDSPVPAQNRSISIPDQDRFWLSAGTTYAFNKDASVDVGVSYMHGQSVKINEGPYQFESEGKAWLFGTNFNYAF

SEQ ID NO: 80 - protein, 398 aa, Artificial Sequence
  Kbl 2-amino-3-ketobutyrate CoA ligase
MRGEFYQQLTNDLETARAEGLFKEERIITSAQQADITVADGSHVINFCANNYLGLANHPDLIAAAKAGMDSHGFGMASVRFICGTQDSHKELEQKLAAFLGMEDAILYSSCFDANGGLFETLLGAEDAIISDALNHASIIDGVRLCKAKRYRYANNDMQELEARLKEAREAGARHVLIATDGVFSMDGVIANLKGVCDLADKYDALVMVDDSHAVGFVGENGRGSHEYCDVMGRVDIITGTLGKALGGASGGYTAARKEVVEWLRQRSRPYLFSNSLAPAIVAASIKVLEMVEAGSELRDRLWANARQFREQMSAAGFTLAGADHAIIPVMLGDAVVAQKFARELQKEGIYVTGFFYPVVPKGQARIRTQMSAAHTPEQITRAVEAFTRIGKQLGVIA

SEQ ID NO: 81 - protein, 341 aa, Artificial Sequence
  Tdh Threonine dehydrogenase
MKALSKLKAEEGIWMTDVPVPELGHNDLLIKIRKTAICGTDVHIYNWDEWSQKTIPVPMVVGHEYVGEVVGIGQEVKGFKIGDRVSGEGHITCGHCRNCRGGRTHLCRNTIGVGVNRPGCFAEYLVIPAFNAFKIPDNISDDLAAIFDPFGNAVHTALSFDLVGEDVLVSGAGPIGIMAAAVAKHVGARNVVITDVNEYRLELARKMGITRAVNVAKENLNDVMAELGMTEGFDVGLEMSGAPPAFRTMLDTMNHGGRIAMLGIPPSDMSIDWTKVIFKGLFIKGIYGREMFETWYKMAALIQSGLDLSPIITHRFSIDDFQKGFDAMRSGQSGKVILSWD

SEQ ID NO: 82 - protein, 248 aa, Artificial Sequence
  YdfG NADP-dependent 3-hydroxy acid dehydrogenase
MIVLVTGATAGFGECITRRFIQQGHKVIATGRRQERLQELKDELGDNLYIAQLDVRNRAAIEEMLASLPAEWCNIDILVNNAGLALGMEPAHKASVEDWETMIDTNNKGLVYMTRAVLPGMVERNHGHIINIGSTAGSWPYAGGNVYGATKAFVRQFSLNLRTDLHGTAVRVTDIEPGLVGGTEFSNVRFKGDDGKAEKTYQNTVALTPEDVSEAVWWVSTLPAHVNINTLEMMPVTQSYAGLNVHRQ